SEQUENCE LISTING (58 sequences)

SEQ ID NO: 1 - protein, 1296 aa, Clostridium botulinum serotype A
MPFVNKQFNYKDPVNGVDIAYIKIPNAGQMQPVKAFKIHNKIWVIPERDTFTNPEEGDLNPPPEAKQVPVSYYDSTYLSTDNEKDNYLKGVTKLFERIYSTDLGRMLLTSIVRGIPFWGGSTIDTELKVIDTNCINVIQPDGSYRSEELNLVIIGPSADIIQFECKSFGHEVLNLTRNGYGSTQYIRFSPDFTFGFEESLEVDTNPLLGAGKFATDPAVTLAHELIHAGHRLYGIAINPNRVFKVNTNAYYEMSGLEVSFEELRTFGGHDAKFIDSLQENEFRLYYYNKFKDIASTLNKAKSIVGTTASLQYMKNVFKEKYLLSEDTSGKFSVDKLKFDKLYKMLTEIYTEDNFVKFFKVLNRKTYLNFDKAVFKINIVPKVNYTIYDGFNLRNTNLAANFNGQNTEINNMNFTKLKNFTGLFEFYKLLCVRGIITSKTKSLDKGYNKALNDLCIKVNNWDLFFSPSEDNFTNDLNKGEEITSDTNIEAAEENISLDLIQQYYLTFNFDNEPENISIENLSSDIIGQLELMPNIERFPNGKKYELDKYTMFHYLRAQEFEHGKSRIALTNSVNEALLNPSRVYTFFSSDYVKKVNKATEAAMFLGWVEQLVYDFTDETSEVSTTDKIADITIIIPYIGPALNIGNMLYKDDFVGALIFSGAVILLEFIPEIAIPVLGTFALVSYIANKVLTVQTIDNALSKRNEKWDEVYKYIVTNWLAKVNTQIDLIRKKMKEALENQAEATKAIINYQYNQYTEEEKNNINFNIDDLSSKLNESINKAMININKFLNQCSVSYLMNSMIPYGVKRLEDFDASLKDALLKYIYDNRGTLIGQVDRLKDKVNNTLSTDIPFQLSKYVDNQRLLSTFTEYIKNIINTSILNLRYESNHLIDLSRYASKINIGSKVNFDPIDKNQIQLFNLESSKIEVILKNAIVYNSMYENFSTSFWIRIPKYFNSISLNNEYTIINCMENNSGWKVSLNYGEIIWTLQDTQEIKQRVVFKYSQMINISDYINRWIFVTITNNRLNNSKIYINGRLIDQKPISNLGNIHASNNIMFKLDGCRDTHRYIWIKYFNLFDKELNEKEIKDLYDNQSNSGILKDFWGDYLQYDKPYYMLNLYDPNKYVDVNNVGIRGYMYLKGPRGSVMTTNIYLNSSLYRGTKFIIKKYASGNKDNIVRNNDRVYINVVVKNKEYRLATNASQAGVEKILSALEIPDVGNLSQVVVMKSKNDQGITNKCKMNLQDNNGNDIGFIGFHQFNNIAKLVASNWYNRQIERSSRTLGCSWEFIPVDDGWGERPL

SEQ ID NO: 2 - protein, 1291 aa, Clostridium botulinum serotype B
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCKSVKAPGICIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 3 - protein, 1291 aa, Clostridium botulinum serotype C1
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCHKAIDGRSLYNKTLDCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 4 - protein, 1276 aa, Clostridium botulinum serotype D
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCLRLTKNSRDDSTCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 5 - protein, 1252 aa, Clostridium botulinum serotype E
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 6 - protein, 1274 aa, Clostridium botulinum serotype F
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCKSVIPRKGTKAPPRLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 7 - protein, 1297 aa, Clostridium botulinum serotype G
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCKPVMYKNTGKSEQCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 8 - protein, 1315 aa, Clostridium teteni
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCKKIIPPTNIRENLYNRTASLTDLGGELCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 9 - protein, 1268 aa, Clostridium baratii
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCKSIVSKKGTKNSLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 10 - protein, 1251 aa, Clostridium butyricum
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCKNIVSVKGIRKSICIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

SEQ ID NO: 11 - protein, 25 aa, Artificial Sequence
  BoNT/A di-chain loop region
CVRGIITSKTKSLDKGYNKALNDLC

SEQ ID NO: 12 - protein, 10 aa, Artificial Sequence
  BoNT/B di-chain loop region
CKSVKAPGIC

SEQ ID NO: 13 - protein, 17 aa, Artificial Sequence
  BoNT/C1 di-chain loop region
CHKAIDGRSLYNKTLDC

SEQ ID NO: 14 - protein, 14 aa, Artificial Sequence
  BoNT/D di-chain loop region
CLRLTKNSRDDSTC

SEQ ID NO: 15 - protein, 15 aa, Artificial Sequence
  BoNT/E di-chain loop region
CKNIVSVKGIRKSIC

SEQ ID NO: 16 - protein, 17 aa, Artificial Sequence
  BoNT/F di-chain loop region
CKSVIPRKGTKAPPRLC

SEQ ID NO: 17 - protein, 15 aa, Artificial Sequence
  BoNT/G di-chain loop region
CKPVMYKNTGKSEQC

SEQ ID NO: 18 - protein, 29 aa, Artificial Sequence
  TeNT di-chain loop region
CKKIIPPTNIRENLYNRTASLTDLGGELC

SEQ ID NO: 19 - protein, 15 aa, Artificial Sequence
  BaNT di-chain loop region
CKSIVSKKGTKNSLC

SEQ ID NO: 20 - protein, 15 aa, Artificial Sequence
  BuNT di-chain loop region
CKNIVSVKGIRKSIC

SEQ ID NO: 21 - protein, 5 aa, Artificial Sequence
  G-spacer
GGGGS

SEQ ID NO: 22 - protein, 5 aa, Artificial Sequence
  A-spacer
EAAAK

SEQ ID NO: 23 - protein, 8 aa, Artificial Sequence
  FLAG epitope-binding region
DYKDDDDK

SEQ ID NO: 24 - protein, 9 aa, Artificial Sequence
  Human Influenza virus hemagluttinin (HA) epitope-binding region
YPYDVPDYA

SEQ ID NO: 25 - protein, 10 aa, Artificial Sequence
  Human p62 c-Myc epitope-binding region
EQKLISEEDL

SEQ ID NO: 26 - protein, 11 aa, Artificial Sequence
  Vesicular Stomatitis Virus Glycoprotein (VSV-G) epitope-binding region
YTDIEMNRLGK

SEQ ID NO: 27 - protein, 6 aa, Artificial Sequence
  Substance P epitope-binding region
QFFGLM

SEQ ID NO: 28 - protein, 11 aa, Artificial Sequence
  Glycoprotein-D precursor of Herpes simplex virus epitope-binding region
QPELAPEDPED

SEQ ID NO: 29 - protein, 14 aa, Artificial Sequence
  V5 epitope-binding region
GKPIPNPLLGLDST

SEQ ID NO: 30 - protein, 6 aa, Artificial Sequence
  AU1 epitope-binding region
DTYRYI

SEQ ID NO: 31 - protein, 6 aa, Artificial Sequence
  AU5 epitope-binding region
TDFYLK

SEQ ID NO: 32 - protein, 6 aa, Artificial Sequence
  HIS epitope-binding region
HHHHHH

SEQ ID NO: 33 - protein, 527 aa, Clostridium botulinum serotype A
MYIMLKNKLSILLTSTLVAASLLSFKPVYADSVNNSNKTNLNNVRQEIQKAKNNNQKVTIMYYCDADNNLESSLLSDIEEMKKGYVNNPNLNLVTLIDRSSRYTSDKTVFGEDFEDARLYKIEHNKTKRLDGGKEFPEITLNSNYEANMGDADTLKKFINYCKANYKADKYVLIMANHGGGAKEKLKNNQDVNRAICWDDSHYDGNSPDCLYMGEISDTLTQEQSVDVLAFDACLMGTAEVAYQFRPGNGGFSADGIVASSPVVWGPGFQYDNILSRLKSGGGSSNEDDLTLGGKEKNFDPATITNEQLGALFVEEQRDSTHARGSYDQHLSFYDAAKVEDVKRSIDNLAINLSNENKKDGIEKLRGSKNNTNLMHYFDEYNEQEWIEYPYFDIYDLCEKINESNNFSNETRTLASICMDKIDEMVVYSFGGPSKEFKEGKNGLSIFLPDGDKKYSNYYNSIPHWTIQSWYNSIDTVQNGLPPYGKLTWCQDGQDPRVNKVGNWFELLDSWFDKTNGADGGVNHYQW

SEQ ID NO: 34 - protein, 526 aa, Clostridium histolyticum
MLRRKVSTLLMTALITTSFLNSKPVYANPVTKSKDNNLKEVQQVTSKSNKNKNQKVTIMYYCDADNNLEGSLLNDIEEMKTGYKDSPNLNLIALVDRSPRYSSDEKVLGEDFSDTRLYKIEHNKANRLDGKNEFPEISTTSKYEANMGDPEVLKKFIDYCKSNYEADKYVLIMANHGGGAREKSNPRLNRAICWDDSNLDKNGEADCLYMGEISDHLTEKQSVDLLAFDACLMGTAEVAYQYRPGNGGFSADTLVASSPVVWGPGFKYDKIFDRIKAGGGTNNEDDLTLGGKEQNFDPATITNEQLGALFVEEQRDSTHANGRYDQHLSFYDLKKAESVKRAIDNLAVNLSNENKKSEIEKLRGSGIHTDLMHYFDEYSEGEWVEYPYFDVYDLCEKINKSENFSSKTKDLASNAMNKLNEMIVYSFGDPSNNFKEGKNGLSIFLPNGDKKYSTYYTSTKIPHWTMQSWYNSIDTVKYGLNPYGKLSWCKDGQDPEINKVGNWFELLDSWFDKTNDVTGGVNHYQW

SEQ ID NO: 35 - protein, 530 aa, Clostridium novyi
MLRKKVIHLLAAAILGMSFINIQPVYATPKPTANNAKSSITKKAKDESKKEQKVTILYYCDADNNLEDALMSDIAEMKKGYVNNPNLNLIALVDRTPGYSEDSTALGENFEDTRLYKIEHNKATRLDGGKYFPEIKVNGTYEANMGDPETLKKFIEFGKNNYKADKYVLIMSNHGGGAKNKPNVNEKLNKAICWDDSNLDGENPDCLYIGEISDHLDESHSVDVLAFDACLMGTAEVAYQYRPGNGRFSAKTMIASSPVVWGAGFKYDDIFSRIRSGNTFTLQKDLTLGGRERCFDPATITNEQIGALFVEEQRDSTSRYFRSDQQLSCYDLTKAENIKKSFDKLAVDLSTKNKKSAIEKLRGSKTNVNLMHYFNEKDQLDWIEYPYFDIYDLCEGISKSNDFDEETQKLAKDVMKNVDSMVLYSFGGKKFKGVGKFKEGKNGLSVFLPDGNRIYTSRYSNAKIPHWLIQSWYNSIDTVASGLNNPYGKLSWCKDGQDPEINKVGNWFELLDSWFDKTNGPDGGFNHYQW

SEQ ID NO: 36 - protein, 522 aa, Clostridium perfringens
MFKKKLSLLMATITIGSVLLGGASTVSAAPRQKHKTVSEKIKEAEKTEGDKKLTVMVYADCDNNLEEYILNDIEEMKEGYKNNPNLNIVVLVDRIPGYSNDSKVLGSNFEDTRLYKIGENSAERISGKSEFPEITTTSNYEANMGDANTLKKFIKFCKKNYEADKYMLIMSNHGGGAKDDKDRASTVNKAICWDDSNNKDCLYTGEISDVLTKDESVDVLVFDACLMGTSEVAYQYRPNNGSFEAKTLVASAPVVWGNGYPYDKIFSRLKSTKGDNGEVDSTLGGKEKIFEPSLVTNNELGALFVEEQRDSVNSYGVTDQQLSCYDLSKIETVKKSVDALARNLSKNNKKDAIENLRGTGKNAPTMHYFKNYDEYEWIEYPYFDLYDLCEKISLSNEFDETTKKLSKNVMKNVDQLILYSFAGNDFKGFKEGKNGISIFLPDGNRNYYDQYSGQVIPHWAIQRWYNPLDTNAYRLRSGYGKLAWCKDGLDPKINKVGNWFELLDSWFDKDNTSLGGYNRYRY

SEQ ID NO: 37 - protein, 522 aa, Clostridium perfringens
MFKKKLSLLMATITIGSVLLGGVSTVSAAPRQKHKTVSEKIKEAEKTEGDKKLTVMVYADCDNNLEEYILNDIEEMKEGYKNNPNLNIIVLVDRIPGYSNDSKVLGSNFEDTRLYKIGENSAERISGKSEFPEITTTSNYEANMGDANTLKKFIKFCKKNYEADKYMLIMSNHGGGAKDDKDRASTVNKAICWDDSNNKDCLYTGEISDVLTKDESVDVLVFDACLMGNSEVAYQYRPNNGSFEAKTLVASAPVVWGFGYPYDKIFSRLRSTKGDNGEVDSTLGGKEKIFDPSTVTNNELGALFVEEQRDSVNSCGVTDQQLSCYDLSKIEKVKKSVDTLARNLSKNNKKDAIESLRGTGKNAPTMHYFKNYDEYEWIEYPYFDLYDLCEKISLSDEFNETTKKLSKNVMKNVDQLILYSFAGNDFKGFKEGKNGISIFLPDGNRNYYDQYSGQAIPHWAIQRWYNPLDTNAYRLRSGYGKLSWCKDGLDPKINKVGNWFELLDSWFDKDNTSLGGYNRYRY

SEQ ID NO: 38 - protein, 524 aa, Clostridium perfringens
MYMFKKKLSLLMATITIGSVLLGGVSTVSAAPRQKHKTVSEKIKEAEKTEGDKKLTVMVYADCDNNLEEYILNDIEEMKEGYKNNPNLNIIVLVDRIPGYSNDSKVLGSNFEDTRLYKIGENSAERISGKSEFPEITTTSNYEANMGDANTLKKFIKFCKKNYEADKYMLIMSNHGGGAKDDKDRASTVNKAICWDDSNNKDCLYTGEISDVLTKDESVDVLVFDACLMGNSEVAYQYRPNNGSFEAKTLVASAPVVWGFGYPYDKIFSRLRSTKGDNGEVDSTLGGKEKIFDPSTVTNNELGALFVEEQRDSVNSCRVTDQQLSCYDLSKIEKVKKSVDALARNLSKNNKKDAIEKLRGTGKNAPTMHYFKNYDEYEWIEYPYFDLYDLCEKISLSDEFNETTKKLSKNVMKNVDQLILYSFAGKDFKGFKEGKNGISIFLPDGNRNYYDQYSGQAIPHWAIQRWYNPLDTNAYRLRSGYGKLAWCKDGLDPKINKVGNWFELLDSWFDKDNTSLGGYNRYRY

SEQ ID NO: 39 - protein, 772 aa, Clostridium botulinum serotype A
MLRKIESKDLLYNLELDDINIDRTQKYTPEYNSIYEKINLALDIDKSGYNLYLVDDFSKEKLNSIINFINQKLEKKSKPKDICYVVLEEERYPYSIYLENGKGKILKEKLKDIQTKYNECIYDFYNKSSNKEKEIILESMEKKRSEIVNELIEESKKEGFEIKTGVSGFVFMPIKDGNSLSESEYEDLNKEDKEEILEKVSKLKEKAESSLEVLADMEREGLEKLKDIMRTYLEMEMKGSKEEYRMEFEDNIQTLDFLNSVCRNIEKELIESYTSSYEEDQESIINVIYKYKVNVIVDNTLNKSPLVIFEENPSVNNLVGSIEYENKSGVYYTDASLIKAGSLLKANEGCLIVRANSLFTNGSAYFYLKKALINDKIDFDYNKGYLELLSLGGLKPEPINTKLKVIIIGDYETYNLLYNYDEDFKKIFKLKSEYNKVVDINSKSKEQICKNIYDICENKDLKNINEEAVKEVCKYLSRKAENKNKFYFDNYEIDRLLIQADSKARIEDRDIITKEDIQFVAYEKEEIEKEVMEGYEKERIFIDVKGDKVGQVNGLSVIDLGYASFGRPIRITCCCYKGNGDIIDIQKESNLSGNIHNKAISTLKGYINSIIGKYDTLPVDFHLSFEQIYGTVDGDSASVAEAIAMLSALSNIPVRQSIAVTGSINQFGQVQPIGGVNEKIEGFYEVCRYKKDIKDKGILIPKSNKENLVLNKEVEEAIKNGEFSIYTMETLEDAVKILLGEKNLKFNELIVEIEKELKKYNKKSKKCRKIKL

SEQ ID NO: 40 - protein, 575 aa, Clostridium botulinum serotype A
MKSKKLLTILLSAIITASSISSVYAAESVGIKSKYQPKTTTIFWEKGKQNNKKSATNIASEQFNNFEEINQFFQQNISRFGLKKGSLKSTKALKDEKGKTHYHTIYQVEGIPVYYGRIVFTTEKDSTISSINGGVDISFENENWKNKIKLSKSDAIAKAKNNIKYEELHDSKADLYLYNFEEKPYVVYLIDLSTDTGDWNVFVNAEDGSIVNKFNNIPTLTNTRDKKFTSTKKTNTKVNKSNNLIDVQGNTIKGKGKSSLNGIVDIDLTYKDGKYYLKNSNKNIYVYDLNNKYINTFTTPKSSILKASKLVENNNNEFIDDKHIIAVDAYINLEKTYDYYKNKFNRNSIDNKGMNVEAFIHHGEKYAGAEWSENLGSMLLGDGDGRNSSHMSKALDVVGHEFSHGVTRKESNLKYENESGALNESFSDIMGIAIKGKNFKLGEDCWTPDIEGDAIRDMQDPSKGYQPAHMKDYRSMDIRYDNGGVHVNSGIINHAAYLIADGIEKLGVENSKDIMAKLFYTANCYEWDETTNFSKCRNDLIKVTKDLYGENSKYVQIVENAFDKVGITATPQLPL

SEQ ID NO: 41 - protein, 592 aa, Clostridium botulinum serotype A
MKSKKLLATVLSAVITFSAVSAVSAAPVGKESKSEPKITTISWDKSEQNTKKDATDIKQKKFNNAQEVTNFFEKNISKFGVKKGTLKSTKTLKDDKGKTHYHTIYEVEGIPVYYGRIVFTTEKDSTMNSINGRVDTVFENGNWKNKIKLSKEDAIAKAKGDIKDQKSNSEKADLYLYNFEGKPYVVYLVNTMTDSGNWNVFVNAEDGSIVNKFDTTPTLVENKDKKLPNAKKIKDEAEKNEAKKANASNVNSVTDVQGQSVKGMGRTSLDGLVNLDLTYGSGRYYLKDNNRKIYLYDLKNQVSGDDLYRYIIEHYYYGAPEYKQRLMSQSELVSNSNNNFINDNQVNSVDAYVNTAKTYDYYKNKLSRNSIDNKGMNVNGFVHVDKNLGNAFWYGPYDSMFFGDGDGVRFSALAKSLDVVGHELSHGVTNKQSNLNYANESGALNESFSDIMGTAVEGKNFVLGEDCWIAGGVMRDMENPSRGNQPAHMKDYVYMSEDNGGVHKNSGIINHAAYLIADGFEKMGAKDSKDIMGKLFYIANCYYWDQTTDFAKCRNDVVRVAKDLYGENSKEVQIVKNAFDKVGVSATPQLSL

SEQ ID NO: 42 - protein, 594 aa, Clostridium botulinum serotype A
MKSKKLLATVLSAVITFSAVSAVSAAPVGKESKSDPKTTTISWDKSEQNAKKATTDIKQKKFDNAQEITKFFEKNISKFGVKKGTLNSTKTLKDDKGKTHYHTIYEVEGIPVYYGRIVFTTEKDSTMDSINGRVDTVFENGNWKNKIKLSKEDAIAKAKADIKNEKSNKEKAELYLYNFEGKPYVVYLVNSITNSGNWDIFVNAEDGSIVNKFNNTPTLLDTKAEKLPNAKKIKDEAEKNEAKKANNMNNIIDVQGQSVKGLGRTSLDGLVNINLTYDNGRYYLKDNNRKIYLYDLKNQVDLDDLNDFYDSPKGGHNEELMRRSELVSNSNNNFVDDNQVNSVDAYANMAKSYDYYKNKLSRNSLDNKGMNIKGFVHFDKNLGNAFWVGEYDSMFFGDGDGVRLSPLAKALDVVGHELSHGVTNKQSDLKYEKESGALNESFSDIMGTAIEGKNFEIGEDCWIPSDRYGEIMRDMKDPSRGNQPAHMKDFRDLPVDEDHDWGGVHTNSGIINHAAYLIADGFEKMGEKDSKDIMAKIFYIANCYYWDQITDFSKCRNDVVKVAKDLYGDNSKEVQIVKNAFDQVGITATPQLPL

SEQ ID NO: 43 - protein, 581 aa, Clostridium botulinum serotype A
MKSKKLLATVLSAVITFSTVSAVYAAPVGKESKVEPKTTTITWEKNEQNTKKAATDITEKKFNNSEEITKFFEKNISKFGVQKGSLKNTKTVKDEKGKTNYHMIYEVEGIPVYYGRIVFTTEKDSSMDSINGRIDTVFENGNWKNKIKLSKEDAIAKAKNDIKDEKATSKKTDLYLYNFEGKPYVVYLVDLITDNGSWTVFVNAEDGSIVNKFNNTPTLIDTKDQKLPNAKKIKDEAKKASNANNVIDVQGQSVKGVGKTSLDGLVNIDVTYGNGKYYLKDSNKNIYLYDLKNQVDEYDLYNYLSRPNYKQILMSKSELISNYNNNFIANNQVNSVDAYVNTNKTYDYYKNKLNRNSIDNKGMNINGFVHVGRNYGNAFWYGPYDGMFFGDGDGIYFSSLAKSLDVVGHELSHGVTNKESNLKYENESGALNESFSDIMGVAVEGKNFVLGEDCWVAGGVMRDMENPSRGGQPAHMKDYKYKTMNDDNGGVHTNSGIINHAAYLVADGIEKTGAKNSKDIMGKIFYTANCYKWDETTNFAKCRNDVVQVTKELYGENSNYVKIVEKAFDQVGITATPQLPL

SEQ ID NO: 44 - protein, 580 aa, Clostridium botulinum serotype A
MKSKKLLATVLSAVITFSTVSAVSAAPVGKESKSEPKTTTIFWEKSEQNTKKSTTSITQEKFSNSDEITKFFEKNISKFGVKKGSLKNTKAVKDDKGKTHYHMIYQVEDIPVYYGRIVFTTKKDSSLDSINGRIDTAFENESWKDKVKLSKDNAIEKAKSSIKYDNLSKSDADLYLYNFEGKPYVVYLVDLVTDDGDWNVFVNAEDGSIVNKFNNTPTLIDNKDQKLPNAEEIKRSSEKAPNINSVINVNGQSTKGQGKTSLDGIVDIDLTYKDGKYYLKDSNKNIYLYNLNNSWAPTIGIFSKDYILRRSTLVENNSNKFTEDKHVTAVDGYVNLSKTYDYYKNKFNRNSIDNKGMNVEGFIHTGKNFNNAFWRDDLGSMFFGDGDGVKFSSFASALDVVGHEVSHGITSKESKLKYEKESGALNESFSDIMGVAIEGKNFQIGEDCYTPNIPGDALRDMEDPSKGNQPAHMKDFQYLPNDKDHDWGGVHTNSGIINHAAYLIADGMEKSGEKNSKDIMAKLFYRANCYKWDETTNFAKCRNDVVQVTKDLYGENSKYVKIVENAFDQVGITATPQLPL

SEQ ID NO: 45 - protein, 1209 aa, Clostridium botulinum serotype A
MKKKFIKALCSVALSCMICTSCITKVSAAPTNNTKINSNEINISEDSSKSERIPSTKSKPLGLNATKANTAKYSFNDLNKLSNKEILDLTSRIKWNDISDLFQYNDDSYAFYSNKERVQALIDGLYEKASTYTGTDDKGIDTLVEILRSGFYLGFYNDSLKYLNDRSFQDKCIPAMLAIENNKNFKLGEKGQDTVISALGKLIGNASCNAEVVNKTVPILEQYYREMNKYPKDKLKADAVYSLMKEINYDISQYTYDHNIRDGKNTPWSSKIDPFINTISKFAGISKVTEDNGWIINNGIYYTSKFAIYHSNPSIPHSVIDKCLEILPAYSEQYYIAVERIKEDFNCKDSKGNVVDIDKLLEDGKKHYLPKTYTFDNGKMIIKAGDKVDESKIQRLYWASKEVKSQFHRIIGNDKPLEAGNADDVLTMVIYNSPEEYKLNRTLYGYSVDNGGIYIEGIGTFFTYERTPEESIYSLEELFRHEFTHYLQGRYLVPGLFNEGDFYKGNSGRITWFEEGSAEFFAGSTRTSVLPRKSMVGGLSENPKERFSADKILHSKYDDGWEFYKYGYAFSDYMYNNSKKLFSDLVSTMKNNDVKGYENLIENASKDPNVNKSYQNHMQKLVDNYNNYTIPLVSDDYMKKYSNKSLNEIKSDIESTMNLTNSEITKESSQYFDTYTLKANYTLDSNKGEIDNWNCMNNKVNESLEKLNKLGWGGYKTVTAYFSNPKVNSHNQVEYNVVFHGLLTHNKNFNEAPTIKLDFPKEANTDEKIKFSSEGSTDDGKIVSYAWDLGDGETSSEKNPTHVYKAPGTYTVKLTVTDDKGLKSEKSASINIKKVLTGNAVSEKENNNDYVNANPVYSKDLVSGSVSSSDDRDIFYFNVTKPSDITINAEKINKDKSEFTWLLFSEEDKSNYIAYPNKKLENLFYSTVKIDKPGKYYLVIYKVSGDKSDYRFNIEGDISASTKDDTDKNELVISEKEDNNSFDKANRVCKNQSVIATLDTNDPRDTYYFDALTVGNIEVTMENTDNNSNEFNWLAYSSDNTNNYIGYATKREGNKIIGNFKVDKPGRYYIVAYKTSSNKINYKLNIKGDIDNVPKNDEIYEKESNDSFETANKIMLNTTVLGNLNGKDVRDIYSFDIKEAKDLDIKLNNLNNLGLAWNLYKESDLNNYIAYGSVSGNTIQGKCNVTPGKYYLYVYKYSGDNGNYSLTIK

SEQ ID NO: 46 - protein, 212 aa, Clostridium botulinum serotype A
MLNEFYTKGEEIANAITHGIGALLSIAALVILIVFSAKYGDAWYVTSYSIFGSCLFILYLESTLYHSLQGSKVKKIFRIFDHSSIFLLIAGTYTPFILTSLRDPLGWTIFGIEWGLTLIGIILKVFTTGKYEKLSTAIYIFMGWLIMLHAKKLVLVIPRISLIYLIVGGVIYTVGAFLFMLDDIPYNHPIWHLFVIGGSVFHFFSLFYMIPR

SEQ ID NO: 47 - protein, 216 aa, Clostridium botulinum serotype A
MFKKFRDPVSGLTHLFGAIMSIVGLIVLVNYSIFQDSPLHITVFAIFGASLILLYSASSIYHLVTASKKSIRILRRIDHSMIYVLIAGSYTPICLLALKGTFGLAMLTIIWTLAIIGILVKNFWFSAPRWISTGFYLIMGWLIVVAIFPLSKTLSIGGLFWLIAGGVAYSIGAVIYGTKRPKIASKYFTFHDIFHIFVLLGSLCHFILMLNYIMYM

SEQ ID NO: 48 - protein, 401 aa, Clostridium botulinum serotype A
MKKNKKWIIWTVVIVLVTNIFTFLGTNLVSLYLPNGKVIIGADQYKDILKYQKMFLIRNQIYKYYDGKIDESKMVEGAVKGMTESLNDPYTVFMNAKEYKEFNAQTEGNYSGVGIQIQAKDDKIIVASTFEGSPAKEAGILPKDEIQKVNNTTVSGKELEKAVSIMKGKEGTDVKLQLYRKEKGSFEVTLKRKKIDIPTIKSEMIDNNIGYIQVSMFDENTSKNFKNALNDLKDKGMKSLLLDLRGNPGGLLDECINMASNFIEKGKVVVSTIDKYGSKKEYKSKGGDFIGFPVTILVDEGSASASEVFLGAMKDYNVATSIGKKTFGKGVVQTIIETGDNTALKVTISKYYSPKGVNINHKGITPDMEIDYPEELRKKEYDRKVDPQFNKALNIAKSKIR

SEQ ID NO: 49 - protein, 311 aa, Clostridium botulinum serotype A
MSKDIGKRLIILLSIGVTILVITYAYIYTKPNAYEVLVNNNPVAYVKNKEDFNKIYKEVENNIKKRFNLDMRNNINFENIKVKGDILTSNDFIKKSILENSNIKVTAFKVKFQDEFIGILSNKKEIQDLSKIISKKYSVNIINHIKIKEETISVEEINTIDELAINISKSQKLQNFMNSKRLSRGDINEEIALAMPTNGCITSKFGKRWGKFHKGLDIGAPNGTDIYSSLDGKVIYSGWEEGYGKVIKIQHSSELITIYAHCSNLYVKVGQYVKKGEKIGEVGSTGRSTGPHVHFELRKNNEPCNPLTYIK

SEQ ID NO: 50 - protein, 1262 aa, Artificial Sequence
  BoNT/E-DiA
MPKINSFNYNDPVNDRTILYIKPGGCQEFYKSFNIMKNIWIIPERNVIGTTPQDFHPPTSLKNGDSSYYDPNYLQSDEEKDRFLKIVTKIFNRINNNLSGGILLEELSKANPYLGNDNTPDNQFHIGDASAVEIKFSNGSQDILLPNVIIMGAEPDLFETNSSNISLRNNYMPSNHGFGSIAIVTFSPEYSFRFNDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLDKGYNKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVSWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNAIKTIIESKYNSYTLEEKNELTNKYDIKQIENELNQKVSIAMNNIDRFLTESSISYLMKLINEVKINKLREYDENVKTYLLNYIIQHGSILGESQQELNSMVTDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNAGINQKLAFNYGNANGISDYINKWIFVTITNDRLGDSKLYINGNLIDQKSILNLGNIHVSDNILFKIVNCSYTRYIGIRYFNIFDKELDETEIQTLYSNEPNTNILKDFWGNYLLYDKEYYLLNVLKPNNFIDRRKDSTLSINNIRSTILLANRLYSGIKVKIQRVNNSSTNDNLVRKNDQVYINFVASKTHLFPLYADTATTNKEKTIKISSSGNRFNQVVVMNSVGNNCTMNFKNNNGNNIGLLGFKADTVVASTWYYTHMRDHTNSNGCFWNFISEEHGWQEK

SEQ ID NO: 51 - protein, 1306 aa, Artificial Sequence
  BoNT/B-DiA
MPVTINNFNYNDPIDNNNIIMMEPPFARGTGRYYKAFKITDRIWIIPERYTFGYKPEDFNKSSGIFNRDVCEYYDPDYLNTNDKKNIFLQTMIKLFNRIKSKPLGEKLLEMIINGIPYLGDRRVPLEEFNTNIASVTVNKLISNPGEVERKKGIFANLIIFGPGPVLNENETIDIGIQNHFASREGFGGIMQMKFCPEYVSVFNNVQENKGASIFNRRGYFSDPALILMHELIHVLHGLYGIKVDDLPIVPNEKKFFMQSTDAIQAEELYTFGGQDPSIITPSTDKSIYDKVLQNFRGIVDRLNKVLVCISDPNININIYKNKFKDKYKFVEDSEGKYSIDVESFDKLYKSLMFGFTETNIAENYKIKTRASYFSDSLPPVKIKNLLDNEIYTIEEGFNISDKDMEKEYRGQNKAINKQAYEEISKEHLAVYKIQMCVRGIITSKTKSLDKGYNKALNDLCIDVDNEDLFFIADKNSFSDDLSKNERIEYNTQSNYIENDFPINELILDTDLISKIELPSENTESLTDFNVDVPVYEKQPAIKKIFTDENTIFQYLYSQTFPLDIRDISLTSSFDDALLFSNKVYSFFSMDYIKTANKVVEAGLFAGWVKQIVNDFVIEANKSNTMDKIADISLIVPYIGLALNVGNETAKGNFENAFEIAGASILLEFIPELLIPVVGAFLLESYIDNKNKIIKTIDNALTKRNEKWSDMYGLIVAQWLSTVNTQFYTIKEGMYKALNYQAQALEEIIKYRYNIYSEKEKSNINIDFNDINSKLNEGINQAIDNINNFINGCSVSYLMKKMIPLAVEKLLDFDNTLKKNLLNYIDENKLYLIGSAEYEKSKVNKYLKTIMPFDLSIYTNDTILIEMFNKYNSEILNNIILNLRYKDNNLIDLSGYGAKVEVYDGVELNDKNQFKLTSSANSKIRVTQNQNIIFNSVFLDFSVSFWIRIPKYKNDGIQNYIHNEYTIINCMKNNSGWKISIRGNRIIWTLIDINGKTKSVFFEYNIREDISEYINRWFFVTITNNLNNAKIYINGKLESNTDIKDIREVIANGEIIFKLDGDIDRTQFIWMKYFSIFNTELSQSNIEERYKIQSYSEYLKDFWGNPLMYNKEYYMFNAGNKNSYIKLKKDSPVGEILTRSKYNQNSKYINYRDLYIGEKFIIRRKSNSQSINDDIVRKEDYIYLDFFNLNQEWRVYTYKYFKKEEEKLFLAPISDSDEFYNTIQIKEYDEQPTYSCQLLFKKDEESTDEIGLIGIHRFYESGIVFEEYKDYFCISKWYLKEVKRKPYNLKLGCNWQFIPKDEGWTE

SEQ ID NO: 52 - protein, 1299 aa, Artificial Sequence
  BoNT/C1-DiA
MPITINNFNYSDPVDNKNILYLDTHLNTLANEPEKAFRITGNIWVIPDRFSRNSNPNLNKPPRVTSPKSGYYDPNYLSTDSDKDPFLKEIIKLFKRINSREIGEELIYRLSTDIPFPGNNNTPINTFDFDVDFNSVDVKTRQGNNWVKTGSINPSVIITGPRENIIDPETSTFKLTNNTFAAQEGFGALSIISISPRFMLTYSNATNDVGEGRFSKSEFCMDPILILMHELNHAMHNLYGIAIPNDQTISSVTSNIFYSQYNVKLEYAEIYAFGGPTIDLIPKSARKYFEEKALDYYRSIAKRLNSITTANPSSFNKYIGEYKQKLIRKYRFVVESSGEVTVNRNKFVELYNELTQIFTEFNYAKIYNVQNRKIYLSNVYTPVTANILDDNVYDIQNGFNIPKSNLNVLFMGQNLSRNPALRKVNPENMLYLFTKFCVRGIITSKTKSLDKGYNKALNDLCRELLVKNTDLPFIGDISDVKTDIFLRKDINEETEVIYYPDNVSVDQVILSKNTSEHGQLDLLYPSIDSESEILPGENQVFYDNRTQNVDYLNSYYYLESQKLSDNVEDFTFTRSIEEALDNSAKVYTYFPTLANKVNAGVQGGLFLMWANDVVEDFTTNILRKDTLDKISDVSAIIPYIGPALNISNSVRRGNFTEAFAVTGVTILLEAFPEFTIPALGAFVIYSKVQERNEIIKTIDNCLEQRIKRWKDSYEWMMGTWLSRIITQFNNISYQMYDSLNYQAGAIKAKIDLEYKKYSGSDKENIKSQVENLKNSLDVKISEAMNNINKFIRECSVTYLFKNMLPKVIDELNEFDRNTKAKLINLIDSHNIILVGEVDKLKAKVNNSFQNTIPFNIFSYTNNSLLKDIINEYFNNINDSKILSLQNRKNTLVDTSGYNAEVSEEGDVQLNPIFPFDFKLGSSGEDRGKVIVTQNENIVYNSMYESFSISFWIRINKWVSNLPGYTIIDSVKNNSGWSIGIISNFLVFTLKQNEDSEQSINFSYDISNNAPGYNKWFFVTVTNNMMGNMKIYINGKLIDTIKVKELTGINFSKTITFEINKIPDTGLITSDSDNINMWIRDFYIFAKELDGKDINILFNSLQYTNVVKDYWGNDLRYNKEYYMVNIDYLNRYMYANSRQIVFNTRRNNNDFNEGYKIIIKRIRGNTNDTRVRGGDILYFDMTINNKAYNLFMKNETMYADNHSTEDIYAIGLREQTKDINDNIIFQIQPMNNTYYYASQIFKSNFNGENISGICSIGTYRFRLGGDWYRHNYLVPTVKQGNYASLLESTSTHWGFVPVSE

SEQ ID NO: 53 - protein, 1287 aa, Artificial Sequence
  BoNT/D-DiA
MTWPVKDFNYSDPVNDNDILYLRIPQNKLITTPVKAFMITQNIWVIPERFSSDTNPSLSKPPRPTSKYQSYYDPSYLSTDEQKDTFLKGIIKLFKRINERDIGKKLINYLVVGSPFMGDSSTPEDTFDFTRHTTNIAVEKFENGSWKVTNIITPSVLIFGPLPNILDYTASLTLQGQQSNPSFEGFGTLSILKVAPEFLLTFSDVTSNQSSAVLGKSIFCMDPVIALMHELTHSLHQLYGINIPSDKRIRPQVSEGFFSQDGPNVQFEELYTFGGLDVEIIPQIERSQLREKALGHYKDIAKRLNNINKTIPSSWISNIDKYKKIFSEKYNFDKDNTGNFVVNIDKFNSLYSDLTNVMSEVVYSSQYNVKNRTHYFSRHYLPVFANILDDNIYTIRDGFNLTNKGFNIENSGQNIERNPALQKLSSESVVDLFTKVCVRGIITSKTKSLDKGYNKALNDLCIKVKNNRLPYVADKDSISQEIFENKIITDETNVQNYSDKFSLDESILDGQVPINPEIVDPLLPNVNMEPLNLPGEEIVFYDDITKYVDYLNSYYYLESQKLSNNVENITLTTSVEEALGYSNKIYTFLPSLAEKVNKGVQAGLFLNWANEVVEDFTTNIMKKDTLDKISDVSVIIPYIGPALNIGNSALRGNFNQAFATAGVAFLLEGFPEFTIPALGVFTFYSSIQEREKIIKTIENCLEQRVKRWKDSYQWMVSNWLSRITTQFNHINYQMYDSLSYQADAIKAKIDLEYKKYSGSDKENIKSQVENLKNSLDVKISEAMNNINKFIRECSVTYLFKNMLPKVIDELNKFDLRTKTELINLIDSHNIILVGEVDRLKAKVNESFENTMPFNIFSYTNNSLLKDIINEYFNSINDSKILSLQNKKNALVDTSGYNAEVRVGDNVQLNTIYTNDFKLSSSGDKIIVNLNNNILYSAIYENSSVSFWIKISKDLTNSHNEYTIINSIEQNSGWKLCIRNGNIEWILQDVNRKYKSLIFDYSESLSHTGYTNKWFFVTITNNIMGYMKLYINGELKQSQKIEDLDEVKLDKTIVFGIDENIDENQMLWIRDFNIFSKELSNEDINIVYEGQILRNVIKDYWGNPLKFDTEYYIINDNYIDRYIAPESNVLVLVQYPDRSKLYTGNPITIKSVSDKNPYSRILNGDNIILHMLYNSRKYMIIRDTDTIYATQGGECSQNCVYALKLQSNLGNYGIGIFSIKNIVSKNKYCSQIFSSFRENTMLLADIYKPWRFSFKNAYTPVAVTNYETKLLSTSSFWKFISRDPGWVE

SEQ ID NO: 54 - protein, 1282 aa, Artificial Sequence
  BoNT/F-DiA
MPVAINSFNYNDPVNDDTILYMQIPYEEKSKKYYKAFEIMRNVWIIPERNTIGTNPSDFDPPASLKNGSSAYYDPNYLTTDAEKDRYLKTTIKLFKRINSNPAGKVLLQEISYAKPYLGNDHTPIDEFSPVTRTTSVNIKLSTNVESSMLLNLLVLGAGPDIFESCCYPVRKLIDPDVVYDPSNYGFGSINIVTFSPEYEYTFNDISGGHNSSTESFIADPAISLAHELIHALHGLYGARGVTYEETIEVKQAPLMIAEKPIRLEEFLTFGGQDLNIITSAMKEKIYNNLLANYEKIATRLSEVNSAPPEYDINEYKDYFQWKYGLDKNADGSYTVNENKFNEIYKKLYSFTESDLANKFKVKCRNTYFIKYEFLKVPNLLDDDIYTVSEGFNIGNLAVNNRGQSIKLNPKIIDSIPDKGLVEKIVKFCVRGIITSKTKSLDKGYNKALNDLCIRVNNSELFFVASESSYNENDINTPKEIDDTTNLNNNYRNNLDEVILDYNSQTIPQISNRTLNTLVQDNSYVPRYDSNGTSEIEEYDVVDFNVFFYLHAQKVPEGETNISLTSSIDTALLEESKDIFFSSEFIDTINKPVNAALFIDWISKVIRDFTTEATQKSTVDKIADISLIVPYVGLALNIIIEAEKGNFEEAFELLGVGILLEFVPELTIPVILVFTIKSYIDSYENKNKAIKAINNSLIEREAKWKEIYSWIVSNWLTRINTQFNKRKEQMYQALQNQVDAIKTAIEYKYNNYTSDEKNRLESEYNINNIEEELNKKVSLAMKNIERFMTESSISYLMKLINEAKVGKLKKYDNHVKSDLLNYILDHRSILGEQTNELSDLVTSTLNSSIPFELSSYTNDKILIIYFNRLYKKIKDSSILDMRYENNKFIDISGYGSNISINGNVYIYSTNRNQFGIYNSRLSEVNIAQNNDIIYNSRYQNFSISFWVRIPKHYKPMNHNREYTIINCMGNNNSGWKISLRTVRDCEIIWTLQDTSGNKENLIFRYEELNRISNYINKWIFVTITNNRLGNSRIYINGNLIVEKSISNLGDIHVSDNILFKIVGCDDETYVGIRYFKVFNTELDKTEIETLYSNEPDPSILKNYWGNYLLYNKKYYLFNLLRKDKYITLNSGILNINQQRGVTEGSVFLNYKLYEGVEVIIRKNGPIDISNTDNFVRKNDLAYINVVDRGVEYRLYADTKSEKEKIIRTSNLNDSLGQIIVMDSIGNNCTMNFQNNNGSNIGLLGFHSNNLVASSWYYNNIRRNTSSNGCFWSSISKENGWKE

SEQ ID NO: 55 - protein, 1307 aa, Artificial Sequence
  BoNT/G-DiA
MPVNIKNFNYNDPINNDDIIMMEPFNDPGPGTYYKAFRIIDRIWIVPERFTYGFQPDQFNASTGVFSKDVYEYYDPTYLKTDAEKDKFLKTMIKLFNRINSKPSGQRLLDMIVDAIPYLGNASTPPDKFAANVANVSINKKIIQPGAEDQIKGLMTNLIIFGPGPVLSDNFTDSMIMNGHSPISEGFGARMMIRFCPSCLNVFNNVQENKDTSIFSRRAYFADPALTLMHELIHVLHGLYGIKISNLPITPNTKEFFMQHSDPVQAEELYTFGGHDPSVISPSTDMNIYNKALQNFQDIANRLNIVSSAQGSGIDISLYKQIYKNKYDFVEDPNGKYSVDKDKFDKLYKALMFGFTETNLAGEYGIKTRYSYFSEYLPPIKTEKLLDNTIYTQNEGFNIASKNLKTEFNGQNKAVNKEAYEEISLEHLVIYRIAMCVRGIITSKTKSLDKGYNKALNDLCIIVNNEDLFFIANKDSFSKDLAKAETIAYNTQNNTIENNFSIDQLILDNDLSSGIDLPNENTEPFTNFDDIDIPVYIKQSALKKIFVDGDSLFEYLHAQTFPSNIENLQLTNSLNDALRNNNKVYTFFSTNLVEKANTVVGASLFVNWVKGVIDDFTSESTQKSTIDKVSDVSIIIPYIGPALNVGNETAKENFKNAFEIGGAAILMEFIPELIVPIVGFFTLESYVGNKGHIIMTISNALKKRDQKWTDMYGLIVSQWLSTVNTQFYTIKERMYNALNNQSQAIEKIIEDQYNRYSEEDKMNINIDFNDIDFKLNQSINLAINNIDDFINQCSISYLMNRMIPLAVKKLKDFDDNLKRDLLEYIDTNELYLLDEVNILKSKVNRHLKDSIPFDLSLYTKDTILIQVFNNYISNISSNAILSLSYRGGRLIDSSGYGATMNVGSDVIFNDIGNGQFKLNNSENSNITAHQSKFVVYDSMFDNFSINFWVRTPKYNNNDIQTYLQNEYTIISCIKNDSGWKVSIKGNRIIWTLIDVNAKSKSIFFEYSIKDNISDYINKWFSITITNDRLGNANIYINGSLKKSEKILNLDRINSSNDIDFKLINCTDTTKFVWIKDFNIFGRELNATEVSSLYWIQSSTNTLKDFWGNPLRYDTQYYLFNQGMQNIYIKYFSKASMGETAPRTNFNNAAINYQNLYLGLRFIIKKASNSRNINNDNIVREGDYIYLNIDNISDESYRVYVLVNSKEIQTQLFLAPINDDPTFYDVLQIKKYYEKTTYNCQILCEKDTKTFGLFGIGKFVKDYGYVWDTYDNYFCISQWYLRRISENINKLRLGCNWQFIPVDEGWTE

SEQ ID NO: 56 - protein, 1311 aa, Artificial Sequence
  TeNT-DiA
MPITINNFRYSDPVNNDTIIMMEPPYCKGLDIYYKAFKITDRIWIVPERYEFGTKPEDFNPPSSLIEGASEYYDPNYLRTDSDKDRFLQTMVKLFNRIKNNVAGEALLDKIINAIPYLGNSYSLLDKFDTNSNSVSFNLLEQDPSGATTKSAMLTNLIIFGPGPVLNKNEVRGIVLRVDNKNYFPCRDGFGSIMQMAFCPEYVPTFDNVIENITSLTIGKSKYFQDPALLLMHELIHVLHGLYGMQVSSHEIIPSKQEIYMQHTYPISAEELFTFGGQDANLISIDIKNDLYEKTLNDYKAIANKLSQVTSCNDPNIDIDSYKQIYQQKYQFDKDSNGQYIVNEDKFQILYNSIMYGFTEIELGKKFNIKTRLSYFSMNHDPVKIPNLLDDTIYNDTEGFNIESKDLKSEYKGQNMRVNTNAFRNVDGSGLVSKLIGLCVRGIITSKTKSLDKGYNKALNDLCIKIKNEDLTFIAEKNSFSEEPFQDEIVSYNTKNKPLNFNYSLDKIIVDYNLQSKITLPNDRTTPVTKGIPYAPEYKSNAASTIEIHNIDDNTIYQYLYAQKSPTTLQRITMTNSVDDALINSTKIYSYFPSVISKVNQGAQGILFLQWVRDIIDDFTNESSQKTTIDKISDVSTIVPYIGPALNIVKQGYEGNFIGALETTGVVLLLEYIPEITLPVIAALSIAESSTQKEKIIKTIDNFLEKRYEKWIEVYKLVKAKWLGTVNTQFQKRSYQMYRSLEYQVDAIKKIIDYEYKIYSGPDKEQIADEINNLKNKLEEKANKAMININIFMRESSRSFLVNQMINEAKKQLLEFDTQSKNILMQYIKANSKFIGITELKKLESKINKVFSTPIPFSYSKNLDCWVDNEEDIDVILKKSTILNLDINNDIISDISGFNSSVITYPDAQLVPGINGKAIHLVNNESSEVIVHKAMDIEYNDMFNNFTVSFWLRVPKVSASHLEQYGTNEYSIISSMKKHSLSIGSGWSVSLKGNNLIWTLKDSAGEVRQITFRDLPDKFNAYLANKWVFITITNDRLSSANLYINGVLMGSAEITGLGAIREDNNITLKLDRCNNNNQYVSIDKFRIFCKALNPKEIEKLYTSYLSITFLRDFWGNPLRYDTEYYLIPVASSSKDVQLKNITDYMYLTNAPSYTNGKLNIYYRRLYNGLKFIIKRYTPNNEIDSFVKSGDFIKLYVSYNNNEHIVGYPKDGNAFNNLDRILRVGYNAPGIPLYKKMEAVKLRDLKTYSVQLKLYDDKNASLGLVGTHNGQIGNDPNRDILIASNWYFNHLKDKILGCDWYFVPTDEGWTND

SEQ ID NO: 57 - protein, 1278 aa, Artificial Sequence
  BaNT-DiA
MPVNINNFNYNDPINNTTILYMKMPYYEDSNKYYKAFEIMDNVWIIPERNIIGKKPSDFYPPISLDSGSSAYYDPNYLTTDAEKDRFLKTVIKLFNRINSNPAGQVLLEEIKNGKPYLGNDHTAVNEFCANNRSTSVEIKESNGTTDSMLLNLVILGPGPNILECSTFPVRIFPNNIAYDPSEKGFGSIQLMSFSTEYEYAFNDNTDLFIADPAISLAHELIHVLHGLYGAKGVTNKKVIEVDQGALMAAEKDIKIEEFITFGGQDLNIITNSTNQKIYVILLSNYTAIASRLSQVNRNNSALNTTYYKNFFQWKYGLDQDSNGNYTVNISKFNAIYKKLFSFTECDLAQKFQVKNRSNYLFHFKPFRLLDLLDDNIYSISEGFNIGSLRVNNNGQNINLNSRIVGPIPDNGLVERFVGLCVRGIITSKTKSLDKGYNKALNDLCIKVNNRDLFFVASESSYNENGINSPKEIDDTTITNNNYKKNLDEVILDYNSDAIPNLSSRLLNTTAQNDSYVPKYDSNGTSEIKEYTVDKLNVFFYLYAQKAPEGESAISLTSSVNTALLDASKVYTFFSSDFINTVNKPVQAALFISWIQQVINDFTTEATQKSTIDKIADISLIVPYVGLALNIGNEVQKGNFKEAIELLGAGILLEFVPELLIPTILVFTIKSFINSDDSKNKIIKAINNALRERELKWKEVYSWIVSNWLTRINTQFNKRKEQMYQALQNQVDGIKKIIEYKYNNYTLDEKNRLRAEYNIYSIKEELNKKVSLAMQNIDRFLTESSISYLMKLINEAKINKLSEYDKRVNQYLLNYILENSSTLGTSSVPELNNLVSNTLNNSIPFELSEYTNDKILIHILIRFYKRIIDSSILNMKYENNRFIDSSGYGSNISINGDIYIYSTNRNQFGIYSSRLSEVNITQNNTIIYNSRYQNFSVSFWVRIPKYNNLKNLNNEYTIINCMRNNNSGWKISLNYNNIIWTLQDTTGNNQKLVFNYTQMIDISDYINKWTFVTITNNRLGHSKLYINGNLTDQKSILNLGNIHVDDNILFKIVGCNDTRYVGIRYFKIFNMELDKTEIETLYHSEPDSTILKDFWGNYLLYNKKYYLLNLLKPNMSVTKNSDILNINRQRGIYSKTNIFSNARLYTGVEVIIRKVGSTDTSNTDNFVRKNDTVYINVVDGNSEYQLYADVSTSAVEKTIKLRRISNSNYNSNQMIIMDSIGDNCTMNFKTNNGNDIGLLGFHLNNLVASSWYYKNIRNNTRNNGCFWSFISKEHGWQE

SEQ ID NO: 58 - protein, 1261 aa, Artificial Sequence
  BuNT-DiA
MPTINSFNYNDPVNNRTILYIKPGGCQQFYKSFNIMKNIWIIPERNVIGTIPQDFLPPTSLKNGDSSYYDPNYLQSDQEKDKFLKIVTKIFNRINDNLSGRILLEELSKANPYLGNDNTPDGDFIINDASAVPIQFSNGSQSILLPNVIIMGAEPDLFETNSSNISLRNNYMPSNHGFGSIAIVTFSPEYSFRFKDNSMNEFIQDPALTLMHELIHSLHGLYGAKGITTKYTITQKQNPLITNIRGTNIEEFLTFGGTDLNIITSAQSNDIYTNLLADYKKIASKLSKVQVSNPLLNPYKDVFEAKYGLDKDASGIYSVNINKFNDIFKKLYSFTEFDLATKFQVKCRQTYIGQYKYFKLSNLLNDSIYNISEGYNINNLKVNFRGQNANLNPRIITPITGRGLVKKIIRFCVRGIITSKTKSLDKGYNKALNDLCIEINNGELFFVASENSYNDDNINTPKEIDDTVTSNNNYENDLDQVILNFNSESAPGLSDEKLNLTIQNDAYIPKYDSNGTSDIEQHDVNELNVFFYLDAQKVPEGENNVNLTSSIDTALLEQPKIYTFFSSEFINNVNKPVQAALFVGWIQQVLVDFTTEANQKSTVDKIADISIVVPYIGLALNIGNEAQKGNFKDALELLGAGILLEFEPELLIPTILVFTIKSFLGSSDNKNKVIKAINNALKERDEKWKEVYSFIVSNWMTKINTQFNKRKEQMYQALQNQVNALKAIIESKYNSYTLEEKNELTNKYDIEQIENELNQKVSIAMNNIDRFLTESSISYLMKLINEVKINKLREYDENVKTYLLDYIIKHGSILGESQQELNSMVIDTLNNSIPFKLSSYTDDKILISYFNKFFKRIKSSSVLNMRYKNDKYVDTSGYDSNININGDVYKYPTNKNQFGIYNDKLSEVNISQNDYIIYDNKYKNFSISFWVRIPNYDNKIVNVNNEYTIINCMRDNNSGWKVSLNHNEIIWTLQDNSGINQKLAFNYGNANGISDYINKWIFVTITNDRLGDSKLYINGNLIDKKSILNLGNIHVSDNILFKIVNCSYTRYIGIRYFNIFDKELDETEIQTLYNNEPNANILKDFWGNYLLYDKEYYLLNVLKPNNFINRRTDSTLSINNIRSTILLANRLYSGIKVKIQRVNNSSTNDNLVRKNDQVYINFVASKTHLLPLYADTATTNKEKTIKISSSGNRFNQVVVMNSVGNCTMNFKNNNGNNIGLLGFKADTVVASTWYYTHMRDNTNSNGFFWNFISEEHGWQEK

(19) United States
(12) Patent Application Publication
(10) Pub. No.: US 2013/0245227 A1
(43) Pub. Date: Sep. 19, 2013

(54) METHODS OF ACTIVATING CLOSTRIDIAL TOXINS

(71) Applicant: ALLERGAN, INC., Irvine, CA (US)

(72) Inventors: Marc F. Verhagen, Irvine, CA (US); Dean G. Stathakis, Irvine, CA (US); Lance E. Steward, Irvine, CA (US)

(73) Assignee: Allergan, Inc., Irvine, CA (US)

(21) Appl. No.: 13/907,345

(22) Filed: May 31, 2013

Related U.S. Application Data
(63) Continuation of application No. 12/669,447, filed on Jan. 15, 2010, now Pat. No. 8,486,422.

(51) Int. Cl. C07K 14/33

(52) U.S. Cl. CPC C07K 14/33
USPC 530/350; 536/23.7; 435/69.1

(57) ABSTRACT

The specification discloses modified Clostridial toxins comprising an exogenous Clostridial toxin di-chain loop protease cleavage site located within the di-chain loop region; polynucleotide molecules encoding such modified Clostridial toxins; method of producing such modified Clostridial toxins, method of activating such modified Clostridial toxins and methods of activating recombinantly-expressed Clostridial toxins.

This application is a continuation of and claims priority pursuant to 35 U.S.C. §120 to U.S. patent application Ser. No. 12/669,447, filed Jan. 15, 2010, which claims priority pursuant to 35 U.S.C. 371 to application PCT/US08/68504, filed Jun. 27, 2008, which claims priority pursuant to 35 U.S.C. §119(e) to U.S. Provisional Patent Application Ser. No. 60/952,112 filed Jul. 26, 2007, all incorporated entirely by reference.

The ability of Clostridial toxins, such as, e.g., Botulinum neurotoxins (BoNTs), BoNT/A, BoNT/B, BoNT/C1, BoNT/D, BoNT/E, BoNT/F and BoNT/G, Tetanus neurotoxin (TeNT), Baratium neurotoxin (BaNT) and Butyricum neurotoxin (BuNT) to inhibit neuronal transmission are being exploited in a wide variety of therapeutic and cosmetic applications, see e.g., William J. Lipham, COSMETIC AND CLINICAL APPLICATIONS OF BOTULINUM TOXIN (Slack, Inc., 2004). Clostridial toxins commercially available as pharmaceutical compositions include, BoNT/A preparations, such as, e.g., BOTOX® (Allergan, Inc., Irvine, Calif.), Dysport®/Reloxin®, (Beaufour Ipsen, Porton Down, England), Linurase® (Prollenium, Inc., Ontario, Canada), Neuronox® (Medy-Tox, Inc., Ochang-myeon, South Korea) BTX-A (Lanzhou Institute Biological Products, China) and Xeomin® (Merz Pharmaceuticals, GmbH., Frankfurt, Germany); and BoNT/B preparations, such as, e.g., MyoBloc™/NeuroBloc™ (Elan Pharmaceuticals, San Francisco, Calif.). As an example, BOTOX® is currently approved in one or more countries for the following indications: achalasia, adult spasticity, anal fissure, back pain, blepharospasm, bruxism, cervical dystonia, essential tremor, glabellar lines or hyperkinetic facial lines, headache, hemifacial spasm, hyperactivity of bladder, hyperhidrosis, juvenile cerebral palsy, multiple sclerosis, myoclonic disorders, nasal labial lines, spasmodic dysphonia, strabismus and VII nerve disorder.

The increasing use of Clostridial toxin therapies in treating a wider range of human afflictions necessitates increasing the efficiency with which these toxins are produced. However, meeting the needs for the ever increasing demand for such toxin treatments may become difficult. One outstanding problem is that all Clostridial toxins need to be converted into the di-chain form of the molecule in order to achieve optimal activity. Historically, this conversion has been done in one of two ways. The first method simply purifies a Clostridial toxin di-chain from the bacterial strain itself, thereby relying on the naturally-occurring endogenous protease used to convert the single-chain form of the toxin into the di-chain form. The second method utilizes an exogenous protease that converts the single-chain form into the di-chain by either taking advantage of a fortuitous cleavage site found in the appropriate location or by genetically engineering a protease cleavage site of commonly used, commercially available exogenous proteases. However, there are several drawbacks to both of these methods. For example, methods employing an endogenous protease produce low toxin yields because native Clostridial strains usually produce little toxin. In addition these strains are poorly suited for research, thus hindering the efforts to genetic manipulation Clostridial toxins to improve their therapeutic and cosmetic attributes. Lastly, several Clostridial strains do not produce the endogenous protease necessary to convert the single-chain form of the toxin to the di-chain form. A drawback to the use of exogenous proteases is a lack of protease specificity that results in inactive toxin because of proteolytic cleavage in inappropriate locations. In addition, many of the currently available proteases are from animal sources that lack Good Manufacture Standard (GMS) approval, requiring additional purification steps during the manufacturing process. Thus, methods currently used to convert the single-chain form of the toxin into the di-chain form are inefficient, cumbersome and/or lead to higher overall production costs. These drawbacks represent a significant obstacle to the overall commercial production of Clostridial toxins and are thus a major problem since di-chain forms of these toxins are needed for scientific, therapeutic and cosmetic applications. In addition, both the amount of Clostridial toxins anticipated for future therapies and the demand for toxins with enhanced therapeutic properties are increasing. Therefore, there is a need to develop better methods for producing Clostridial toxin di-chain molecules in order to meet this need.

The present invention provides modified Clostridial toxins that rely on a novel method of converting the single-chain form of the toxin into the di-chain form and novel methods of convering single-chain Clostridial toxins. These and related advantages are useful for various clinical, therapeutic and cosmetic applications, such as, e.g., the treatment of neuromuscular disorders, neuropathic disorders, eye disorders, pain, muscle injuries, headache, cardiovascular diseases, neuropsychiatric disorders, endocrine disorders, cancers, otic disorders and hyperkinetic facial lines, as well as, other disorders where a Clostridial toxin administration to a mammal can produce a beneficial effect.

### BRIEF DESCRIPTION OF THE DRAWINGS

FIG. 1 shows a schematic of the current paradigm of Clostridial toxin posttranslational processing. Clostridial toxins are translated as a single-chain polypeptide of approximately 150 kDa comprising an enzymatic domain, a translocation domain and a binding domain. A disulfide bridge formed from a cysteine residue in the enzymatic domain and a cysteine residue from the translocation domain form a di-chain loop. Within this di-chain loop is a protease cleavage site for a naturally-occurring protease that can be produced endogenously from the Clostridial strain synthesizing the toxin, or exogenously from a source found in the environment. Cleavage of the protease cleavage site by the naturally-occurring protease converts the single-chain form of the toxin into the di-chain form. The di-chain form of the toxin is held together by the disulfide bond and non-covalent interactions between the two chains.

FIGS. 2a and 2b show a schematic of the current paradigm of neurotransmitter release and Clostridial toxin intoxication in a central and peripheral neuron. FIG. 2a shows a schematic for the neurotransmitter release mechanism of a central and peripheral neuron. The release process can be described as comprising two steps: 1) vesicle docking, where the vesicle-bound SNARE protein of a vesicle containing neurotransmitter molecules associates with the membrane-bound SNARE proteins located at the plasma membrane; and 2) neurotransmitter release, where the vesicle fuses with the plasma membrane and the neurotransmitter molecules are exocytosed. FIG. 2b shows a schematic of the intoxication mechanism for tetanus and botulinum toxin activity in a central and peripheral neuron. This intoxication process can be described as comprising four steps: 1) receptor binding, where a Clostridial toxin binds to a Clostridial receptor system and initiates the intoxication process; 2) complex internalization, where after toxin binding, a vesicle containing the toxin/receptor system complex is endocytosed into the cell; 3) light chain translocation, where multiple events result in the release of the active light chain into the cytoplasm; and 4) enzymatic target modification, where the active light chain of Clostridial toxin proteolytically cleaves its target SNARE substrate, such as, e.g., SNAP-25, VAMP or Syntaxin, thereby preventing vesicle docking and neurotransmitter release.

### DETAILED DESCRIPTION

Clostridia toxins produced by Clostridium botulinum, Clostridium tetani, Clostridium baratii and Clostridium butyricum are the most widely used in therapeutic and cosmetic treatments of humans and other mammals. Strains of C. botulinum produce seven antigenically-distinct types of Botulinum toxins (BoNTs), which have been identified by investigating botulism outbreaks in man (BoNT/A, /B, /E and /F), animals (BoNT/C1 and /D), or isolated from soil (BoNT/G). BoNTs possess approximately 35% amino acid identity with each other and share the same functional domain organization and overall structural architecture. It is recognized by those of skill in the art that within each type of Clostridial toxin there can be subtypes that differ somewhat in their amino acid sequence, and also in the nucleic acids encoding these proteins. For example, there are presently four BoNT/A subtypes, BoNT/A1, BoNT/A2, BoNT/A3 and BoNT/A4, with specific subtypes showing approximately 89% amino acid identity when compared to another BoNT/A subtype. While all seven BoNT serotypes have similar structure and pharmacological properties, each also displays heterogeneous bacteriological characteristics. In contrast, tetanus toxin (TeNT) is produced by a uniform group of C. tetani. Two other species of Clostridia, C. baratii and C. butyricum, also produce toxins, BaNT and BuNT respectively, which are similar to BoNT/F and BoNT/E, respectively.

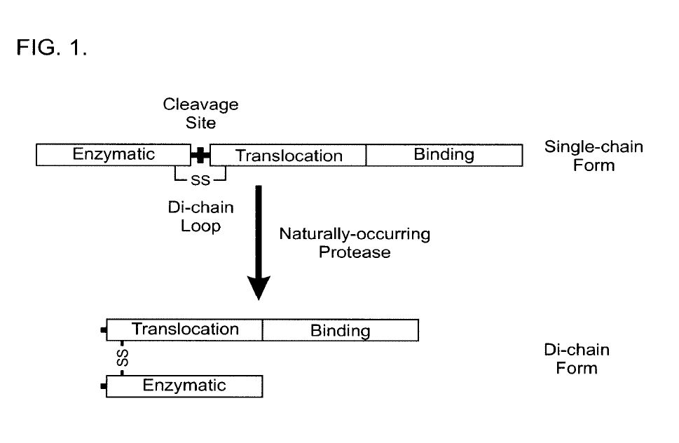

Clostridial toxins are each translated as a single chain polypeptide of approximately 150 kDa that is subsequently cleaved by proteolytic scission within a disulfide loop by a naturally-occurring protease (FIG. 1). This cleavage occurs within the discrete di-chain loop region created between two cysteine residues that form a disulfide bridge. This posttranslational processing yields a di-chain molecule comprising an approximately 50 kDa light chain (LC) and an approximately 100 kDa heavy chain (HC) held together by the single disulfide bond and non-covalent interactions between the two chains. The naturally-occurring protease used to convert the single chain molecule into the di-chain is currently not known. In some bacterial serotypes, such as, e.g., a BoNT/A, a BoNT/B proteolytic, a BoNT/F proteolytic, a BaNT proteolytic strain, or a TeNT, the naturally-occurring protease is produced endogenously by the bacteria serotype and cleavage occurs within the cell before the toxin is release into the environment. However, in other bacterial serotypes, such as, e.g., a BoNT/B nonproteolytic, a BoNT/C1, a BoNT/D, a BoNT/E, a BoNT/F nonproteolytic, a BoNT/G, a BaNT nonproteolytic, or a BuNT, the bacterial strain appears not to produce appreciable amounts of an endogenous protease capable of converting the single chain form of the toxin into the di-chain form. In these situations, the toxin is released from the cell as a single-chain toxin which is subsequently converted into the di-chain form by a naturally-occurring protease found in the environment.

The present invention discloses novel methods that can convert the single-chain polypeptide form of a recombinantly-expressed Clostridial toxin or a modified Clostridial toxins into the di-chain form using the enzymatic activity of a di-chain loop protease isolated from a Clostridial bacteria strain. The present specification discloses several proteases having proteolytic activity for the cleavage site within the di-chain loop region. Thus discovery has lead to the development of methods of activating recombinantly-expressed Clostridial toxins, irrespective of whether these recombinantly-expressed Clostridial toxins are 1) Clostridial toxins capable of cleavage by a di-chain protease expressed within the Clostridial bacterial strain expressing that toxin, such as, e.g., a BoNT/A, a BoNT/B proteolytic, a BoNT/F proteolytic, a BaNT proteolytic, or a TeNT bacterial strain; or 2) modified Clostridial toxins comprising a cleavage site within the di-chain loop region that can be cleaved the di-chain proteases disclosed, such as, e.g., a Clostridial toxin from a BoNT/B nonproteolytic, a BoNT/C1, a BoNT/D, a BoNT/E, a BoNT/F nonproteolytic, a BoNT/G, a BaNT nonproteolytic, or BuNT, bacterial strain modified to include the di-chain loop region from a Clostridial toxin produced by a BoNT/A, a BoNT/B proteolytic or a BoNT/F proteolytic bacterial strain.

As a non-limiting example, BoNT/A expressed naturally from Clostridia botulinum serotype A strain is produced in its di-chain polypeptide form. This is because the single-chain polypeptide is converted into its di-chain form by a di-chain loop protease produced by the bacterium. However, when a BoNT/A is recombinantly expresses, such as, e.g., in an E. coli bacterial strain, this conversion does not occur since E. coli strains do not express the di-chain loop protease. As a result, recombinantly expresses BoNT/A is primarily isolated in its single-chain polypeptide form, a form that is approximately 100 times less active than the di-chain form. Thus, the presently disclosed methods of activating recombinantly-expressed Clostridial toxins include methods that convert a recombinantly expressed single-chain BoNT/A into its di-chain form using a BoNT/A di-chain protease disclosed in the present specification.

As another non-limiting example, BoNT/E expressed naturally from Clostridia botulinum serotype E strain is produced in its single-chain polypeptide form. This is because the C. botulinum serotype E bacterium does not express a di-chain loop protease capable of cleaving the toxin within the di-chain loop region. Similarly, when a BoNT/E is recombinantly expresses, such as, e.g., in an E. coli bacterial strain, this conversion does not occur since E. coli strains also do not express the di-chain loop protease. Thus, the present specification discloses modified Clostridial toxins comprising a di-chain loop protease cleavage site from a Clostridial toxin expressed in a Clostridia botulinum strain expressing an endogenous di-chain loop protease, such as, e.g., a modified BoNT/E comprising a BoNT/A di-chain loop region including a BoNT/A di-chain loop protease cleavage site. This can be accomplished, for instance, by replacing the naturally-occurring di-chain loop region from BoNT/E with a di-chain loop region including the di-chain loop protease cleavage site from BoNT/A. Using such a modified BoNT/E, the presently disclosed methods of activating recombinantly-expressed Clostridial toxins include methods that convert a recombinantly expressed single-chain modified BoNT/E into its di-chain form using a BoNT/A di-chain protease disclosed in the present specification.

Aspects of the present invention provide modified Clostridial toxins comprising an exogenous Clostridial toxin di-chain loop including a Clostridial toxin di-chain loop protease cleavage site from a different Clostridial toxin. It is envisioned that the exogenous di-chain loop region can replace the endogenous di-chain loop region or be in addition to the endogenous di-chain loop region. It is also envisioned that any Clostridial toxin di-chain loop region including a di-chain loop protease cleavage site can be used. including, without limitation, a BoNT/A di-chain loop region including a di-chain loop protease cleavage site, a BoNT/B di-chain loop region including a di-chain loop protease cleavage site, a BoNT/C1 di-chain loop region including a di-chain loop protease cleavage site, a BoNT/D di-chain loop region including a di-chain loop protease cleavage site, a BoNT/E di-chain loop region including a di-chain loop protease cleavage site, a BoNT/F di-chain loop region including a di-chain loop protease cleavage site, a BoNT/G di-chain loop region including a di-chain loop protease cleavage site, a TeNT di-chain loop region including a di-chain loop protease cleavage site, a BaNT di-chain loop region including a di-chain loop protease cleavage site and a BuNT di-chain loop region including a di-chain loop protease cleavage site.

Other aspects of the present invention provide polynucleotide molecules encoding modified Clostridial toxins comprising an exogenous Clostridial toxin di-chain loop including a Clostridial toxin di-chain loop protease cleavage site from a different Clostridial toxin.

Other aspects of the present invention provide methods of producing a modified Clostridial toxin comprising an exogenous Clostridial toxin di-chain loop including a Clostridial toxin di-chain loop protease cleavage site from a different Clostridial toxin. Other aspects of the present invention provide methods of producing in a cell a modified Clostridial toxin comprising an exogenous Clostridial toxin di-chain loop including a Clostridial toxin di-chain loop protease cleavage site from a different Clostridial toxin.

Other aspects of the present invention provide methods of activating a modified Clostridial toxin comprising an exogenous Clostridial toxin di-chain loop including a Clostridial toxin di-chain loop protease cleavage site from a different Clostridial toxin.

Yet other aspects of the present invention provide methods of activating a modified Clostridial toxin comprising an exogenous Clostridial toxin di-chain loop including a Clostridial toxin di-chain loop protease cleavage site from a different Clostridial toxin.

Each mature di-chain molecule comprises three functionally distinct domains: 1) an enzymatic domain located in the LC that includes a metalloprotease region containing a zinc-dependent endopeptidase activity which specifically targets core components of the neurotransmitter release apparatus; 2) a translocation domain contained within the amino-terminal half of the HC (H_{N}) that facilitates release of the LC from intracellular vesicles into the cytoplasm of the target cell; and 3) a binding domain found within the carboxyl-terminal half of the HC (H_{C}) that determines the binding activity and binding specificity of the toxin to the receptor complex located at the surface of the target cell. The H_{C} domain comprises two distinct structural features of roughly equal size that indicate function and are designated the H_{CN} and H_{CC} subdomains. Table 1 gives approximate boundary regions for each domain found in exemplary Clostridial toxins.

TABLE 1
Clostridial Toxin Reference Sequences and Regions

| | SEQ | | | | |
|---|---|---|---|---|---|
| | ID | | | H_{C} | |
| Toxin | NO: | LC | H_{N} | H_{CN} | H_{CC} |
| BoNT/ | 1 | M1-K448 | A449-I873 | I874-P1110 | Y1111-L1296 |
| A | | | | | |
| BoNT/ | 2 | M1-K441 | A442-I860 | L861-E1097 | Y1098-E1291 |
| B | | | | | |
| BoNT/ | 3 | M1-K449 | T450-I868 | N869-E1111 | Y1112-E1291 |
| C1 | | | | | |
| BoNT/ | 4 | M1-R445 | D446-I864 | N865-E1098 | Y1099-E1276 |
| D | | | | | |
| BoNT/E | 5 | M1-R422 | K423-I847 | K848-E1085 | Y1086-K1252 |
| BoNT/F | 6 | M1-K439 | A440-I866 | K867-K1105 | Y1106-E1274 |
| BoNT/ | 7 | M1-K446 | S447-I865 | S866-Q1105 | Y1106-E1297 |
| G | | | | | |
| TeNT | 8 | M1-A457 | S458-L881 | K882-E1127 | Y1128-D1315 |
| BaNT | 9 | M1-K431 | N432-I857 | I858-K1094 | Y1095-E1268 |
| BuNT | 10 | M1-R422 | K423-I847 | K848-E1085 | Y1086-K1251 |

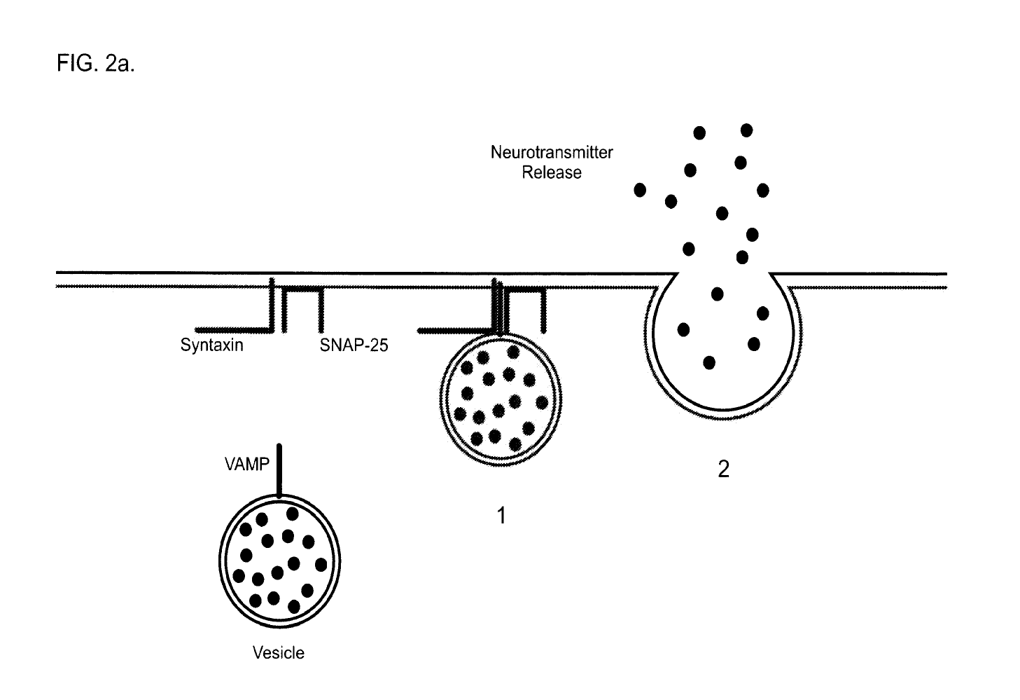

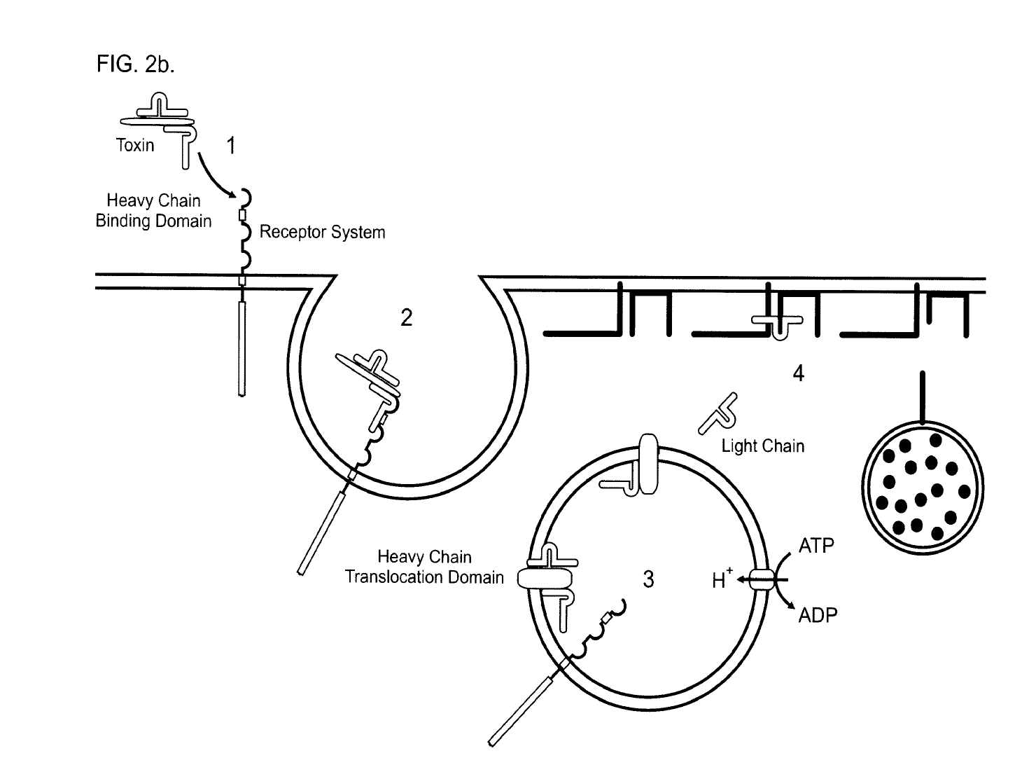

The binding, translocation and enzymatic activity of these three functional domains are all necessary for toxicity. While all details of this process are not yet precisely known, the overall cellular intoxication mechanism whereby Clostridial toxins enter a neuron and inhibit neurotransmitter release is similar, regardless of type. Although the applicants have no wish to be limited by the following description, the intoxication mechanism can be described as comprising at least four steps: 1) receptor binding, 2) complex internalization, 3) light chain translocation, and 4) enzymatic target modification (see FIG. 2). The process is initiated when the H_{C} domain of a Clostridial toxin binds to a toxin-specific receptor complex located on the plasma membrane surface of a target cell. The binding specificity of a receptor complex is thought to be achieved, in part, by specific combinations of gangliosides and protein receptors that appear to distinctly comprise each Clostridial toxin receptor complex. Once bound, the toxin/receptor complexes are internalized by endocytosis and the internalized vesicles are sorted to specific intracellular routes. The translocation step appears to be triggered by the acidification of the vesicle compartment. This process seems to initiate two important pH-dependent structural rearrangements that increase hydrophobicity and promote formation di-chain form of the toxin. Once activated, light chain endopeptidase of the toxin is released from the intracellular vesicle into the cytosol where it specifically targets one of three known core components of the neurotransmitter release apparatus. These core proteins, vesicle-associated membrane protein (VAMP)/synaptobrevin, synaptosomal-associated protein of 25 kDa (SNAP-25) and Syntaxin, are necessary for synaptic vesicle docking and fusion at the nerve terminal and constitute members of the soluble N-ethylmaleimide-sensitive factor-attachment protein-receptor (SNARE) family. BoNT/A and BoNT/E cleave SNAP-25 in the carboxyl-terminal region, releasing a nine or twenty-six amino acid segment, respectively, and BoNT/C1 also cleaves SNAP-25 near the carboxyl-terminus. The botulinum serotypes BoNT/B, BoNT/D, BoNT/F and BoNT/G, and tetanus toxin, act on the conserved central portion of VAMP, and release the amino-terminal portion of VAMP into the cytosol. BoNT/C1 cleaves syntaxin at a single site near the cytosolic membrane surface. The selective proteolysis of synaptic SNAREs accounts for the block of neurotransmitter release caused by Clostridial toxins in vivo. The SNARE protein targets of Clostridial toxins are common to exocytosis in a variety of non-neuronal types; in these cells, as in neurons, light chain peptidase activity inhibits exocytosis, see, e.g., Yann Humeau et al., How Botulinum and Tetanus Neurotoxins Block Neurotransmitter Release, 82(5) Biochimie. 427-446 (2000); Kathryn Turton et al., Botulinum and Tetanus Neurotoxins: Structure, Function and Therapeutic Utility, 27(11) Trends Biochem. Sci. 552-558. (2002); Giovanna Lalli et al., The Journey of Tetanus and Botulinum Neurotoxins in Neurons, 11(9) Trends Microbiol. 431-437, (2003).

Aspects of the present invention provide, in part, a Clostridial toxin. As used herein, the term “Clostridial toxin” means any polypeptide that can execute the overall cellular mechanism whereby a Clostridial toxin enters a neuron and inhibits neurotransmitter release and encompasses the binding of a Clostridial toxin to a low or high affinity receptor complex, the internalization of the toxin/receptor complex, the translocation of the Clostridial toxin light chain into the cytoplasm and the enzymatic modification of a Clostridial toxin substrate.

A Clostridial toxin includes, without limitation, naturally occurring Clostridial toxin variants, such as, e.g., Clostridial toxin isoforms and Clostridial toxin subtypes; non-naturally occurring Clostridial toxin variants, such as, e.g., conservative Clostridial toxin variants, non-conservative Clostridial toxin variants, Clostridial toxin chimeric variants and active Clostridial toxin fragments thereof, or any combination thereof. As used herein, the term “Clostridial toxin variant,” whether naturally-occurring or non-naturally-occurring, means a Clostridial toxin that has at least one amino acid change from the corresponding region of the disclosed reference sequences (see Table 1) and can be described in percent identity to the corresponding region of that reference sequence. As non-limiting examples, a BoNT/A variant comprising amino acids 1-1296 of SEQ ID NO: 1 will have at least one amino acid difference, such as, e.g., an amino acid substitution, deletion or addition, as compared to the amino acid region 1-1296 of SEQ ID NO: 1; a BoNT/B variant comprising amino acids 1-1291 of SEQ ID NO: 2 will have at least one amino acid difference, such as, e.g., an amino acid substitution, deletion or addition, as compared to the amino acid region 1-1291 of SEQ ID NO: 2; a BoNT/C1 variant comprising amino acids 1-1291 of SEQ ID NO: 3 will have at least one amino acid difference, such as, e.g., an amino acid substitution, deletion or addition, as compared to the amino acid region 1-1291 of SEQ ID NO: 3; a BoNT/D variant comprising amino acids 1-1276 of SEQ ID NO: 4 will have at least one amino acid difference, such as, e.g., an amino acid substitution, deletion or addition, as compared to the amino acid region 1-1276 of SEQ ID NO: 4; a BoNT/E variant comprising amino acids 1-1252 of SEQ ID NO: 5 will have at least one amino acid difference, such as, e.g., an amino acid substitution, deletion or addition, as compared to the amino acid region 1-1252 of SEQ ID NO: 5; a BoNT/F variant comprising amino acids 1-1274 of SEQ ID NO: 6 will have at least one amino acid difference, such as, e.g., an amino acid substitution, deletion or addition, as compared to the amino acid region 1-1274 of SEQ ID NO: 6; a BoNT/G variant comprising amino acids 1-1297 of SEQ ID NO: 7 will have at least one amino acid difference, such as, e.g., an amino acid substitution, deletion or addition, as compared to the amino acid region 1-1297 of SEQ ID NO: 7; a TeNT variant comprising amino acids 1-1315 of SEQ ID NO: 8 will have at least one amino acid difference, such as, e.g., an amino acid substitution, deletion or addition, as compared to the amino acid region 1-1315 of SEQ ID NO: 8; a BaNT variant comprising amino acids 1-1268 of SEQ ID NO: 9 will have at least one amino acid difference, such as, e.g., an amino acid substitution, deletion or addition, as compared to the amino acid region 1-1268 of SEQ ID NO: 9; and a BuNT variant comprising amino acids 1-1251 of SEQ ID NO: 10 will have at least one amino acid difference, such as, e.g., an amino acid substitution, deletion or addition, as compared to the amino acid region 1-1251 of SEQ ID NO: 10.

Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

Global methods align sequences from the beginning to the end of the molecule and determine the best alignment by adding up scores of individual residue pairs and by imposing gap penalties. Non-limiting methods include, e.g., CLUSTAL W, see, e.g., Julie D. Thompson et al., CLUSTAL W: Improving the Sensitivity of Progressive Multiple Sequence Alignment Through Sequence Weighting, Position-Specific Gap Penalties and Weight Matrix Choice, 22(22) Nucleic Acids Research 4673-4680 (1994); and iterative refinement, see, e.g., Osamu Gotoh, Significant Improvement in Accuracy of Multiple Protein Sequence Alignments by Iterative Refinement as Assessed by Reference to Structural Alignments, 264(4) J. Mol. Biol. 823-838 (1996).

Local methods align sequences by identifying one or more conserved motifs shared by all of the input sequences. Non-limiting methods include, e.g., Match-box, see, e.g., Eric Depiereux and Ernest Feytmans, Match-Box: A Fundamentally New Algorithm for the Simultaneous Alignment of Several Protein Sequences, 8(5) CABIOS 501-509 (1992); Gibbs sampling, see, e.g., C. E. Lawrence et al., Detecting Subtle Sequence Signals: A Gibbs Sampling Strategy for Multiple Alignment, 262(5131) Science 208-214 (1993); Align-M, see, e.g., Ivo Van Walle et al., Align-M—A New Algorithm for Multiple Alignment of Highly Divergent Sequences, 20(9) Bioinformatics: 1428-1435 (2004).

Hybrid methods combine functional aspects of both global and local alignment methods. Non-limiting methods include, e.g., segment-to-segment comparison, see, e.g., Burkhard Morgenstern et al., Multiple DNA and Protein Sequence Alignment Based On Segment-To-Segment Comparison, 93(22) Proc. Natl. Acad. Sci. U.S.A. 12098-12103 (1996); T-Coffee, see, e.g., Cédric Notredame et al., T-Coffee: A Novel Algorithm for Multiple Sequence Alignment, 302(1) J. Mol. Biol. 205-217 (2000); MUSCLE, see, e.g., Robert C. Edgar, MUSCLE: Multiple Sequence Alignment With High Score Accuracy and High Throughput, 32(5) Nucleic Acids Res. 1792-1797 (2004); and DIALIGN-T, see, e.g., Amarendran R Subramanian et al., DIALIGN-T: An Improved Algorithm for Segment-Based Multiple Sequence Alignment, 6(1) BMC Bioinformatics 66 (2005).

As used herein, the term “naturally occurring Clostridial toxin variant” means any Clostridial toxin produced without the aid of any human manipulation, including, without limitation, Clostridial toxin isoforms produced from alternatively-spliced transcripts, Clostridial toxin isoforms produced by spontaneous mutation and Clostridial toxin subtypes. Non-limiting examples of a Clostridial toxin isoform include, e.g., BoNT/A isoforms, BoNT/B isoforms, BoNT/C1 isoforms, BoNT/D isoforms, BoNT/E isoforms, BoNT/F isoforms, BoNT/G isoforms, TeNT isoforms, BaNT isoforms, and BuNT isoforms. Non-limiting examples of a Clostridial toxin subtype include, e.g., BoNT/A subtypes BoNT/A1, BoNT/A2, BoNT/A3 and BoNT/A4; BoNT/B subtypes BoNT/B1, BoNT/B2, BoNT/B bivalent and BoNT/B nonproteolytic; BoNT/C1 subtypes BoNT/C1-1 and BoNT/C1-2; BoNT/E subtypes BoNT/E1, BoNT/E2 and BoNT/E3; and BoNT/F subtypes BoNT/F1, BoNT/F2, BoNT/F3 and BoNT/F4.

As used herein, the term “non-naturally occurring Clostridial toxin variant” means any Clostridial toxin produced with the aid of human manipulation, including, without limitation, Clostridial toxins produced by genetic engineering using random mutagenesis or rational design and Clostridial toxins produced by chemical synthesis. Non-limiting examples of non-naturally occurring Clostridial toxin variants include, e.g., conservative Clostridial toxin variants, non-conservative Clostridial toxin variants, Clostridial toxin chimeric variants and active Clostridial toxin fragments.

As used herein, the term “conservative Clostridial toxin variant” means a Clostridial toxin that has at least one amino acid substituted by another amino acid or an amino acid analog that has at least one property similar to that of the original amino acid from the reference Clostridial toxin sequence (Table 1). Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin variant can function in substantially the same manner as the reference Clostridial toxin on which the conservative Clostridial toxin variant is based, and can be substituted for the reference Clostridial toxin in any aspect of the present invention. A conservative Clostridial toxin variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the reference Clostridial toxin on which the conservative Clostridial toxin variant is based. A conservative Clostridial toxin variant can also substitute at least 10 contiguous amino acids, at least 15 contiguous amino acids, at least 20 contiguous amino acids, or at least 25 contiguous amino acids from the reference Clostridial toxin on which the conservative Clostridial toxin variant is based, that possess at least 50% amino acid identity, 65% amino acid identity, 75% amino acid identity, 85% amino acid identity or 95% amino acid identity to the reference Clostridial toxin on which the conservative Clostridial toxin variant is based. Non-limiting examples of a conservative Clostridial toxin variant include, e.g., conservative BoNT/A variants, conservative BoNT/B variants, conservative BoNT/C1 variants, conservative BoNT/D variants, conservative BoNT/E variants, conservative BoNT/F variants, conservative BoNT/G variants, conservative TeNT variants, conservative BaNT variants and conservative BuNT variants.

As used herein, the term “non-conservative Clostridial toxin variant” means a Clostridial toxin in which 1) at least one amino acid is deleted from the reference Clostridial toxin on which the non-conservative Clostridial toxin variant is based; 2) at least one amino acid added to the reference Clostridial toxin on which the non-conservative Clostridial toxin is based; or 3) at least one amino acid is substituted by another amino acid or an amino acid analog that does not share any property similar to that of the original amino acid from the reference Clostridial toxin sequence (Table 1). A non-conservative Clostridial toxin variant can function in substantially the same manner as the reference Clostridial toxin on which the non-conservative Clostridial toxin variant is based, and can be substituted for the reference Clostridial toxin in any aspect of the present invention. A non-conservative Clostridial toxin variant can delete one or more amino acids, two or more amino acids, three or more amino acids, four or more amino acids, five or more amino acids, and ten or more amino acids from the reference Clostridial toxin on which the non-conservative Clostridial toxin variant is based. A non-conservative Clostridial toxin variant can add one or more amino acids, two or more amino acids, three or more amino acids, four or more amino acids, five or more amino acids, and ten or more amino acids to the reference Clostridial toxin on which the non-conservative Clostridial toxin variant is based. A non-conservative Clostridial toxin variant may substitute one or more amino acids, two or more amino acids, three or more amino acids, four or more amino acids, five or more amino acids, ten or more amino acids, 20 or more amino acids, 30 or more amino acids, 40 or more amino acids, 50 or more amino acids, 100 or more amino acids, 200 or more amino acids, 300 or more amino acids, 400 or more amino acids, or 500 or more amino acids from the reference Clostridial toxin on which the non-conservative Clostridial toxin variant is based. A non-conservative Clostridial toxin variant can also substitute at least 10 contiguous amino acids, at least 15 contiguous amino acids, at least 20 contiguous amino acids, or at least 25 contiguous amino acids from the reference Clostridial toxin on which the non-conservative Clostridial toxin variant is based, that possess at least 50% amino acid identity, 65% amino acid identity, 75% amino acid identity, 85% amino acid identity or 95% amino acid identity to the reference Clostridial toxin on which the non-conservative Clostridial toxin variant is based. Non-limiting examples of a non-conservative Clostridial toxin variant include, e.g., non-conservative BoNT/A variants, non-conservative BoNT/B variants, non-conservative BoNT/C1 variants, non-conservative BoNT/D variants, non-conservative BoNT/E variants, non-conservative BoNT/F variants, non-conservative BoNT/G variants, non-conservative TeNT variants, non-conservative BaNT variants and non-conservative BuNT variants.

As used herein, the term “Clostridial toxin chimeric variant” means a molecule comprising at least a portion of a Clostridial toxin and at least a portion of at least one other protein to form a toxin with at least one property different from the reference Clostridial toxins of Table 1. One class of Clostridial toxin chimeric variant comprises a modified Clostridial toxin were the endogenous cell binding domain of a naturally-occurring Clostridial toxin is either modified or replaced with a cell binding domain of another molecule. Such modified Clostridial toxin possesses an altered cell binding activity because the modified toxin can, e.g., use the same receptor present on the surface of a naturally occurring Clostridial toxin target cell, referred to as an enhanced cell binding activity for a naturally-occurring Clostridial toxin target cell; use a different receptor present on the surface of a naturally occurring Clostridial toxin target cell, referred to as an altered cell binding activity for a naturally-occurring Clostridial toxin target cell, or use a different receptor present on the surface of the non-Clostridial toxin target cell, referred to as an altered cell binding activity for a non-naturally-occurring Clostridial toxin target cell.

A Clostridial toxin chimeric variant can be a modified Clostridial toxin with an enhanced cell binding activity capable of intoxicating a naturally occurring Clostridial toxin target cell, e.g., a motor neuron. One way this enhanced binding activity is achieved by modifying the endogenous targeting domain of a naturally-occurring Clostridial toxin in order to enhance a cell binding activity of the toxin for its naturally-occurring receptor. Such modifications to a targeting domain result in, e.g., a enhanced cell binding activity that increases binding affinity for an endogenous Clostridial toxin receptor present on a naturally-occurring Clostridial toxin target cell; an enhanced cell binding activity that increases binding specificity for a subgroup of endogenous Clostridial toxin receptors present on a naturally-occurring Clostridial toxin target cell; or an enhanced cell binding activity that increases both binding affinity and binding specificity. Non-limiting examples of modified Clostridial toxins an enhanced cell binding activity for a naturally-occurring Clostridial toxin receptor are described in, e.g., Lance E. Steward, et al., Modified Clostridial Toxins with Enhanced Targeting Capabilities For Endogenous Clostridial Toxin Receptors, International Patent Publication No. 2006/008956 (Mar. 14, 2006), Lance E. Steward, Modified Clostridial Toxins with Enhanced Translocation Capability and Enhanced Targeting Activity, U.S. patent application Ser. No. 11/776,043 (Jul. 11, 2007), each of which is hereby incorporated by reference in its entirety.

A Clostridial toxin chimeric variant can be a modified Clostridial toxin with an altered cell binding activity capable of intoxicating a naturally occurring Clostridial toxin target cell, e.g., a motor neuron. One way this altered capability is achieved by replacing the endogenous targeting domain of a naturally-occurring Clostridial toxin with a targeting domain of another molecule that selectively binds to a different receptor present on the surface of a naturally occurring Clostridial toxin target cell. Such a modification to a targeting domain results in a modified toxin that is able to selectively bind to a non-Clostridial toxin receptor (target receptor) present on a Clostridial toxin target cell. This enhanced binding activity for a naturally occurring Clostridial toxin target cell allows for lower effective doses of a modified Clostridial toxin to be administered to an individual because more toxin will be delivered to the target cell. Thus, modified Clostridial toxins with an enhanced binding activity will reduce the undesirable dispersal of the toxin to areas not targeted for treatment, thereby reducing or preventing the undesirable side-effects associated with diffusion of a Clostridial toxin to an unwanted location. Non-limiting examples of modified Clostridial toxins with an altered cell binding capability for a Clostridial toxin target cell are described in, e.g., Lance E. Steward et al., Modified Clostridial Toxins with Altered Targeting Capabilities For Clostridial Toxin Target Cells, International Patent Publication No. 2006/009831 (Mar. 14, 2005); Lance E. Steward et al., Multivalent Clostridial Toxin Derivatives and Methods of Their Use, U.S. Patent Publication No. 2006/0211619 (Sep. 21, 2006); and Lance E. Steward, Modified Clostridial Toxins with Enhanced Translocation Capabilities and Altered Targeting Activity for Clostridial Toxin Target Cells, U.S. patent application Ser. No. 11/776,052, (Jul. 11, 2007), each of which is hereby incorporated by reference in its entirety.

A Clostridial toxin chimeric variant can be a modified Clostridial toxin with an altered cell binding activity capable of intoxicating a cell other than a naturally occurring Clostridial toxin target cell, e.g., a cell other than a motor neuron. These modified toxins achieve this intoxication by using a target receptor present on non-Clostridial toxin target cell. This re-targeted capability is achieved by replacing a naturally-occurring targeting domain of a Clostridial toxin with a targeting domain showing a selective binding activity for a non-Clostridial toxin receptor present in a non-Clostridial toxin target cell. Such modifications to a targeting domain result in a modified toxin that is able to selectively bind to a non-Clostridial toxin receptor (target receptor) present on a non-Clostridial toxin target cell (re-targeted). A modified Clostridial toxin with an altered targeting activity for a non-Clostridial toxin target cell can bind to a target receptor, translocate into the cytoplasm, and exert its proteolytic effect on the SNARE complex of the non-Clostridial toxin target cell. Non-limiting examples of modified Clostridial toxins with an altered targeting activity for a non-Clostridial toxin target cell are described in, e.g., Keith A. Foster et al., Clostridial Toxin Derivatives Able To Modify Peripheral Sensory Afferent Functions, U.S. Pat. No. 5,989,545 (Nov. 23, 1999); Clifford C. Shone et al., Recombinant Toxin Fragments, U.S. Pat. No. 6,461,617 (Oct. 8, 2002); Conrad P. Quinn et al., Methods and Compounds for the Treatment of Mucus Hypersecretion, U.S. Pat. No. 6,632,440 (Oct. 14, 2003); Lance E. Steward et al., Methods And Compositions For The Treatment Of Pancreatitis, U.S. Pat. No. 6,843,998 (Jan. 18, 2005); Stephan Donovan, Clostridial Toxin Derivatives and Methods For Treating Pain, U.S. Pat. No. 7,138,127 (Nov. 21, 2006); Keith A. Foster et al., Inhibition of Secretion from Non-Neural Cells, U.S. Patent Publication 2003/0180289 (Sep. 25, 2003); J. Oliver Dolly et al., Activatable Recombinant Neurotoxins, U.S. Pat. No. 7,132,259 (Nov. 7, 2006); Keith A. Foster et al., Re-targeted Toxin Conjugates, International Patent Publication WO 2005/023309 (Mar. 17, 2005); Lance E. Steward et al., Multivalent Clostridial Toxin Derivatives and Methods of Their Use, U.S. patent application Ser. No. 11/376,696 (Mar. 15, 2006); Keith A. Foster, Fusion Proteins, International Patent Publication WO 2006/059093 (Jun. 8, 2005); Keith A. Foster, Non-Cytotoxic Protein Conjugates, International Patent Publication WO 2006/059105 (Jun. 8, 2005); and Lance E. Steward, Modified Clostridial Toxins with Enhanced Translocation Capabilities and Altered Targeting Capabilities for Non-Clostridial Toxin Target Cells, U.S. patent application Ser. No. 11/776,075 (Jul. 11, 2007), each of which is hereby incorporated by reference in its entirety. The ability to re-target the therapeutic effects associated with Clostridial toxins has greatly extended the number of medicinal applications able to use a Clostridial toxin therapy. As a non-limiting example, modified Clostridial toxins retargeted to sensory neurons are useful in treating various kinds of chronic pain, such as, e.g., hyperalgesia and allodynia, neuropathic pain and inflammatory pain, see, e.g., Foster, supra, (1999); and Donovan, supra, (2006); and Stephan Donovan, Method For Treating Neurogenic Inflammation Pain with Botulinum Toxin and Substance P Components, U.S. Pat. No. 7,022,329 (Apr. 4, 2006). As another non-limiting example, modified Clostridial toxins retargeted to pancreatic cells are useful in treating pancreatitis, see, e.g., Steward, supra, (2005).

Thus, in an embodiment, a Clostridial toxin chimeric variant can comprise a modified Clostridial toxin disclosed in the present specification where the binding domain comprises an enhanced cell binding activity capable of intoxicating a naturally occurring Clostridial toxin target cell. In another embodiment, a Clostridial toxin chimeric variant can comprise a modified Clostridial toxin disclosed in the present specification where the binding domain comprises an altered cell binding activity capable of intoxicating a naturally occurring Clostridial toxin target cell. In still another embodiment, a Clostridial toxin chimeric variant can comprise a modified Clostridial toxin disclosed in the present specification where the binding domain comprises an altered cell binding activity capable of intoxicating a non-naturally occurring Clostridial toxin target cell.

It is also envisioned that any of a variety of Clostridial toxin fragments can be useful in aspects of the present invention with the proviso that these active fragments can execute the overall cellular mechanism whereby a Clostridial toxin proteolytically cleaves a substrate. Thus, aspects of this embodiment can include Clostridial toxin fragments having a length of, e.g., at least 300 amino acids, at least 400 amino acids, at least 500 amino acids, at least 600 amino acids, at least 700 amino acids, at least 800 amino acids, at least 900 amino acids, at least 1000 amino acids, at least 1100 amino acids and at least 1200 amino acids. Other aspects of this embodiment, can include Clostridial toxin fragments having a length of, e.g., at most 300 amino acids, at most 400 amino acids, at most 500 amino acids, at most 600 amino acids, at most 700 amino acids, at most 800 amino acids, at most 900 amino acids, at most 1000 amino acids, at most 1100 amino acids and at most 1200 amino acids.

It is also envisioned that any of a variety of Clostridial toxin fragments comprising the light chain can be useful in aspects of the present invention with the proviso that these light chain fragments can specifically target the core components of the neurotransmitter release apparatus and thus participate in executing the overall cellular mechanism whereby a Clostridial toxin proteolytically cleaves a substrate. The light chains of Clostridial toxins are approximately 420-460 amino acids in length and comprise an enzymatic domain (Table 1). Research has shown that the entire length of a Clostridial toxin light chain is not necessary for the enzymatic activity of the enzymatic domain. As a non-limiting example, the first eight amino acids of the BoNT/A light chain (residues 1-8 of SEQ ID NO: 1) are not required for enzymatic activity. As another non-limiting example, the first eight amino acids of the TeNT light chain (residues 1-8 of SEQ ID NO: 8) are not required for enzymatic activity. Likewise, the carboxyl-terminus of the light chain is not necessary for activity. As a non-limiting example, the last 32 amino acids of the BoNT/A light chain (residues 417-448 of SEQ ID NO: 1) are not required for enzymatic activity. As another non-limiting example, the last 31 amino acids of the TeNT light chain (residues 427-457 of SEQ ID NO: 8) are not required for enzymatic activity. Thus, aspects of this embodiment can include Clostridial toxin light chains comprising an enzymatic domain having a length of, e.g., at least 350 amino acids, at least 375 amino acids, at least 400 amino acids, at least 425 amino acids and at least 450 amino acids. Other aspects of this embodiment can include Clostridial toxin light chains comprising an enzymatic domain having a length of, e.g., at most 350 amino acids, at most 375 amino acids, at most 400 amino acids, at most 425 amino acids and at most 450 amino acids.

It is also envisioned that any of a variety of Clostridial toxin H_{N} regions comprising a translocation domain can be useful in aspects of the present invention with the proviso that these active fragments can facilitate the release of the LC from intracellular vesicles into the cytoplasm of the target cell and thus participate in executing the overall cellular mechanism whereby a Clostridial toxin proteolytically cleaves a substrate. The H_{N} regions from the heavy chains of Clostridial toxins are approximately 410-430 amino acids in length and comprise a translocation domain (Table 1). Research has shown that the entire length of a H_{N} region from a Clostridial toxin heavy chain is not necessary for the translocating activity of the translocation domain. Thus, aspects of this embodiment can include Clostridial toxin H_{N} regions comprising a translocation domain having a length of, e.g., at least 350 amino acids, at least 375 amino acids, at least 400 amino acids and at least 425 amino acids. Other aspects of this embodiment can include Clostridial toxin H_{N} regions comprising translocation domain having a length of, e.g., at most 350 amino acids, at most 375 amino acids, at most 400 amino acids and at most 425 amino acids.

It is also envisioned that any of a variety of Clostridial toxin H_{C} regions comprising a binding domain can be useful in aspects of the present invention with the proviso that these active fragments can determine the binding activity and binding specificity of the toxin to the receptor complex located at the surface of the target cell execute the overall cellular mechanism whereby a Clostridial toxin proteolytically cleaves a substrate. The H_{C} regions from the heavy chains of Clostridial toxins are approximately 400-440 amino acids in length and comprise a binding domain (Table 1). Research has shown that the entire length of a H_{C} region from a Clostridial toxin heavy chain is not necessary for the binding activity of the binding domain. Thus, aspects of this embodiment can include Clostridial toxin H_{C} regions comprising a binding domain having a length of, e.g., at least 350 amino acids, at least 375 amino acids, at least 400 amino acids and at least 425 amino acids. Other aspects of this embodiment can include Clostridial toxin H_{C} regions comprising a binding domain having a length of, e.g., at most 350 amino acids, at most 375 amino acids, at most 400 amino acids and at most 425 amino acids.

Thus, in an embodiment, a Clostridial toxin comprises a Clostridial toxin enzymatic domain, a Clostridial toxin translocation domain and a Clostridial toxin binding domain. In an aspect of this embodiment, a Clostridial toxin comprises a naturally occurring Clostridial toxin variant, such as, e.g., a Clostridial toxin isoform or a Clostridial toxin subtype. In another aspect of this embodiment, a Clostridial toxin comprises a non-naturally occurring Clostridial toxin variant, such as, e.g., a conservative Clostridial toxin variant, a non-conservative Clostridial toxin variant or an active Clostridial toxin fragment, or any combination thereof. In another aspect of this embodiment, a Clostridial toxin comprises a Clostridial toxin enzymatic domain or an active fragment thereof, a Clostridial toxin translocation domain or an active fragment thereof, a Clostridial toxin binding domain or an active fragment thereof, or any combination thereof. In other aspects of this embodiment, a Clostridial toxin can comprise a BoNT/A, a BoNT/B, a BoNT/C1, a BoNT/D, a BoNT/E, a BoNT/F, a BoNT/G, a TeNT, a BaNT or a BuNT.

In another embodiment, a Clostridial toxin comprises a BoNT/A. In an aspect of this embodiment, a BoNT/A comprises a BoNT/A enzymatic domain, a BoNT/A translocation domain and a BoNT/A binding domain. In another aspect of this embodiment, a BoNT/A comprises SEQ ID NO: 1. In another aspect of this embodiment, a BoNT/A comprises a naturally occurring BoNT/A variant, such as, e.g., a BoNT/A isoform or a BoNT/A subtype. In another aspect of this embodiment, a BoNT/A comprises a naturally occurring BoNT/A variant of SEQ ID NO: 1, such as, e.g., a BoNT/A isoform of SEQ ID NO: 1 or a BoNT/A subtype of SEQ ID NO: 1. In still another aspect of this embodiment, a BoNT/A comprises a non-naturally occurring BoNT/A variant, such as, e.g., a conservative BoNT/A variant, a non-conservative BoNT/A variant or an active BoNT/A fragment, or any combination thereof. In still another aspect of this embodiment, a BoNT/A comprises a non-naturally occurring BoNT/A variant of SEQ ID NO: 1, such as, e.g., a conservative BoNT/A variant of SEQ ID NO: 1, a non-conservative BoNT/A variant of SEQ ID NO: 1 or an active BoNT/A fragment of SEQ ID NO: 1, or any combination thereof. In yet another aspect of this embodiment, a BoNT/A comprises a BoNT/A enzymatic domain or an active fragment thereof, a BoNT/A translocation domain or an active fragment thereof, a BoNT/A binding domain or an active fragment thereof, or any combination thereof. In yet another aspect of this embodiment, a BoNT/A comprising a BoNT/A enzymatic domain of amino acids 1-448 from SEQ ID NO: 1 or an active fragment thereof, a BoNT/A translocation domain of amino acids 449-871 from SEQ ID NO: 1 or an active fragment thereof, a BoNT/A binding domain of amino acids 872-1296 from SEQ ID NO: 1 or an active fragment thereof, and any combination thereof.

In other aspects of this embodiment, a BoNT/A comprises a polypeptide having, e.g., at least 70% amino acid identity with SEQ ID NO: 1, at least 75% amino acid identity with the SEQ ID NO: 1, at least 80% amino acid identity with SEQ ID NO: 1, at least 85% amino acid identity with SEQ ID NO: 1, at least 90% amino acid identity with SEQ ID NO: 1 or at least 95% amino acid identity with SEQ ID NO: 1. In yet other aspects of this embodiment, a BoNT/A comprises a polypeptide having, e.g., at most 70% amino acid identity with SEQ ID NO: 1, at most 75% amino acid identity with the SEQ ID NO: 1, at most 80% amino acid identity with SEQ ID NO: 1, at most 85% amino acid identity with SEQ ID NO: 1, at most 90% amino acid identity with SEQ ID NO: 1 or at most 95% amino acid identity with SEQ ID NO: 1.

In other aspects of this embodiment, a BoNT/A comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 1. In yet other aspects of this embodiment, a BoNT/A comprises a polypeptide having, e.g., at most one, two, three, four, five, six, seven, eight, nine, 10, 20, 30, 40, 50, 100, 200 or 500 non-contiguous amino acid deletions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 non-contiguous amino acid deletions relative to SEQ ID NO: 1. In still other aspects of this embodiment, a BoNT/A comprises a polypeptide having, e.g., at most one, two, three, four, five, six, seven, eight, nine, 10, 20, 30, 40, 50, 100, 200 or 500 non-contiguous amino acid additions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 non-contiguous amino acid additions relative to SEQ ID NO: 1.

In other aspects of this embodiment, a BoNT/A comprises a polypeptide having, e.g., at most one, two, three, four, five, six, seven, eight, nine, 10, 20, 30, 40, 50, 100, 200 or 500 contiguous amino acid substitutions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 contiguous amino acid substitutions relative to SEQ ID NO: 1. In yet other aspects of this embodiment, a BoNT/A comprises a polypeptide having, e.g., at most one, two, three, four, five, six, seven, eight, nine, 10, 20, 30, 40, 50, 100, 200 or 500 contiguous amino acid deletions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 contiguous amino acid deletions relative to SEQ ID NO: 1. In still other aspects of this embodiment, a BoNT/A comprises a polypeptide having, e.g., at most one, two, three, four, five, six, seven, eight, nine, 10, 20, 30, 40, 50, 100, 200 or 500 contiguous amino acid additions relative to SEQ ID NO: 1. In other aspects of this embodiment, a BoNT/A comprises a polypeptide having, e.g., at least one, two, three, four, five, six, seven, eight, nine, 10, 20, 30, 40, 50, 100, 200 or 500 contiguous amino acid additions relative to SEQ ID NO: 1.

In another embodiment, a Clostridial toxin comprises a BoNT/B. In an aspect of this embodiment, a BoNT/B comprises a BoNT/B enzymatic domain, a BoNT/B translocation domain and a BoNT/B binding domain. In another aspect of this embodiment, a BoNT/B comprises SEQ ID NO: 2. In another aspect of this embodiment, a BoNT/B comprises a naturally occurring BoNT/B variant, such as, e.g., a BoNT/β isoform or a BoNT/B subtype. In another aspect of this embodiment, a BoNT/B comprises a naturally occurring BoNT/B variant of SEQ ID NO: 2, such as, e.g., a BoNT/β isoform of SEQ ID NO: 2 or a BoNT/B subtype of SEQ ID NO: 2. In still another aspect of this embodiment, a BoNT/B comprises a non-naturally occurring BoNT/B variant, such as, e.g., a conservative BoNT/B variant, a non-conservative BoNT/B variant or an active BoNT/B fragment, or any combination thereof. In still another aspect of this embodiment, a BoNT/B comprises a non-naturally occurring BoNT/B variant of SEQ ID NO: 2, such as, e.g., a conservative BoNT/B variant of SEQ ID NO: 2, a non-conservative BoNT/B variant of SEQ ID NO: 2 or an active BoNT/B fragment of SEQ ID NO: 2, or any combination thereof. In yet another aspect of this embodiment, a BoNT/B comprising a BoNT/B enzymatic domain or an active fragment thereof, a BoNT/B translocation domain or active fragment thereof, a BoNT/B binding domain or active fragment thereof, and any combination thereof. In yet another aspect of this embodiment, a BoNT/B comprising a BoNT/B enzymatic domain of amino acids 1-441 from SEQ ID NO: 2 or active fragment thereof, a BoNT/B translocation domain of amino acids 442-858 from SEQ ID NO: 2 or active fragment thereof, a BoNT/B binding domain of amino acids 859-1291 from SEQ ID NO: 2 or active fragment thereof, and any combination thereof.

In other aspects of this embodiment, a BoNT/B comprises a polypeptide having, e.g., at least 70% amino acid identity with SEQ ID NO: 2, at least 75% amino acid identity with the SEQ ID NO: 2, at least 80% amino acid identity with SEQ ID NO: 2, at least 85% amino acid identity with SEQ ID NO: 2, at least 90% amino acid identity with SEQ ID NO: 2 or at least 95% amino acid identity with SEQ ID NO: 2. In yet other aspects of this embodiment, a BoNT/B comprises a polypeptide having, e.g., at most 70% amino acid identity with SEQ ID NO: 2, at most 75% amino acid identity with the SEQ ID NO: 2, at most 80% amino acid identity with SEQ ID NO: 2, at most 85% amino acid identity with SEQ ID NO: 2, at most 90% amino acid identity with SEQ ID NO: 2 or at most 95% amino acid identity with SEQ ID NO: 2.

In other aspects of this embodiment, a BoNT/B comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 2. In yet other aspects of this embodiment, a BoNT/B comprises a polypeptide having, e.g., at most one, two, three, four, five, six, seven, eight, nine, 10, 20, 30, 40, 50, 100, 200 or 500 non-contiguous amino acid deletions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 non-contiguous amino acid deletions relative to SEQ ID NO: 2. In still other aspects of this embodiment, a BoNT/B comprises a polypeptide having, e.g., at most one, two, three, four, five, six, seven, eight, nine, 10, 20, 30, 40, 50, 100, 200 or 500 non-contiguous amino acid additions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 non-contiguous amino acid additions relative to SEQ ID NO: 2.

In other aspects of this embodiment, a BoNT/B comprises a polypeptide having, e.g., at most one, two, three, four, five, six, seven, eight, nine, 10, 20, 30, 40, 50, 100, 200 or 500 contiguous amino acid substitutions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 contiguous amino acid substitutions relative to SEQ ID NO: 2. In yet other aspects of this embodiment, a BoNT/B comprises a polypeptide having, e.g., at most one, two, three, four, five, six, seven, eight, nine, 10, 20, 30, 40, 50, 100, 200 or 500 contiguous amino acid deletions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 contiguous amino acid deletions relative to SEQ ID NO: 2. In still other aspects of this embodiment, a BoNT/B comprises a polypeptide having, e.g., at most one, two, three, four, five, six, seven, eight, nine, 10, 20, 30, 40, 50, 100, 200 or 500 contiguous amino acid additions relative to SEQ ID NO: 2. In other aspects of this embodiment, a BoNT/B comprises a polypeptide having, e.g., at least one, two, three, four, five, six, seven, eight, nine, 10, 20, 30, 40, 50, 100, 200 or 500 contiguous amino acid additions relative to SEQ ID NO: 2.

In another embodiment, a Clostridial toxin comprises a BoNT/C1. In an aspect of this embodiment, a BoNT/C1 comprises a BoNT/C1 enzymatic domain, a BoNT/C1 translocation domain and a BoNT/C1 binding domain. In another aspect of this embodiment, a BoNT/C1 comprises SEQ ID NO: 3. In another aspect of this embodiment, a BoNT/C1 comprises a naturally occurring BoNT/C1 variant, such as, e.g., a BoNT/C1 isoform or a BoNT/C1 subtype. In another aspect of this embodiment, a BoNT/C1 comprises a naturally occurring BoNT/C1 variant of SEQ ID NO: 3, such as, e.g., a BoNT/C1 isoform of SEQ ID NO: 3 or a BoNT/C1 subtype of SEQ ID NO: 3. In still another aspect of this embodiment, a BoNT/C1 comprises a non-naturally occurring BoNT/C1 variant, such as, e.g., a conservative BoNT/C1 variant, a non-conservative BoNT/C1 variant or an active BoNT/C1 fragment, or any combination thereof. In still another aspect of this embodiment, a BoNT/C1 comprises a non-naturally occurring BoNT/C1 variant of SEQ ID NO: 3, such as, e.g., a conservative BoNT/C1 variant of SEQ ID NO: 3, a non-conservative BoNT/C1 variant of SEQ ID NO: 3 or an active BoNT/C1 fragment of SEQ ID NO: 3, or any combination thereof. In yet another aspect of this embodiment, a BoNT/C1 comprises a BoNT/C1 enzymatic domain or active fragment thereof, a BoNT/C1 translocation domain or active fragment thereof, a BoNT/C1 binding domain or active fragment thereof, and any combination thereof. In yet another aspect of this embodiment, a BoNT/C1 comprises a BoNT/C1 enzymatic domain of amino acid 1-449 from SEQ ID NO: 3 or active fragment thereof, a BoNT/C1 translocation domain of amino acids 450-866 from SEQ ID NO: 3 or active fragment thereof, a BoNT/C1 binding domain of amino acids 867-1291 from SEQ ID NO: 3 or active fragment thereof, and any combination thereof.

In other aspects of this embodiment, a BoNT/C1 comprises a polypeptide having, e.g., at least 70% amino acid identity with SEQ ID NO: 3, at least 75% amino acid identity with the SEQ ID NO: 3, at least 80% amino acid identity with SEQ ID NO: 3, at least 85% amino acid identity with SEQ ID NO: 3, at least 90% amino acid identity with SEQ ID NO: 3 or at least 95% amino acid identity with SEQ ID NO: 3. In yet other aspects of this embodiment, a BoNT/C1 comprises a polypeptide having, e.g., at most 70% amino acid identity with SEQ ID NO: 3, at most 75% amino acid identity with the SEQ ID NO: 3, at most 80% amino acid identity with SEQ ID NO: 3, at most 85% amino acid identity with SEQ ID NO: 3, at most 90% amino acid identity with SEQ ID NO: 3 or at most 95% amino acid identity with SEQ ID NO: 3.

In other aspects of this embodiment, a BoNT/C1 comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 3. In yet other aspects of this embodiment, a BoNT/C1 comprises a polypeptide having, e.g., at most one, two, three, four, five, six, seven, eight, nine, 10, 20, 30, 40, 50, 100, 200 or 500 non-contiguous amino acid deletions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 non-contiguous amino acid deletions relative to SEQ ID NO: 3. In still other aspects of this embodiment, a BoNT/C1 comprises a polypeptide having, e.g., at most one, two, three, four, five, six, seven, eight, nine, 10, 20, 30, 40, 50, 100, 200 or 500 non-contiguous amino acid additions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 non-contiguous amino acid additions relative to SEQ ID NO: 3.

In other aspects of this embodiment, a BoNT/C1 comprises a polypeptide having, e.g., at most one, two, three, four, five, six, seven, eight, nine, 10, 20, 30, 40, 50, 100, 200 or 500 contiguous amino acid substitutions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 contiguous amino acid substitutions relative to SEQ ID NO: 3. In yet other aspects of this embodiment, a BoNT/C1 comprises a polypeptide having, e.g., at most one, two, three, four, five, six, seven, eight, nine, 10, 20, 30, 40, 50, 100, 200 or 500 contiguous amino acid deletions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 contiguous amino acid deletions relative to SEQ ID NO: 3. In still other aspects of this embodiment, a BoNT/C1 comprises a polypeptide having, e.g., at most one, two, three, four, five, six, seven, eight, nine, 10, 20, 30, 40, 50, 100, 200 or 500 contiguous amino acid additions relative to SEQ ID NO: 3. In other aspects of this embodiment, a BoNT/C1 comprises a polypeptide having, e.g., at least one, two, three, four, five, six, seven, eight, nine, 10, 20, 30, 40, 50, 100, 200 or 500 contiguous amino acid additions relative to SEQ ID NO: 3.

In another embodiment, a Clostridial toxin comprises a BoNT/D. In an aspect of this embodiment, a BoNT/D comprises a BoNT/D enzymatic domain, a BoNT/D translocation domain and a BoNT/D binding domain. In another aspect of this embodiment, a BoNT/D comprises SEQ ID NO: 4. In another aspect of this embodiment, a BoNT/D comprises a naturally occurring BoNT/D variant, such as, e.g., a BoNT/D isoform or a BoNT/D subtype. In another aspect of this embodiment, a BoNT/D comprises a naturally occurring BoNT/D variant of SEQ ID NO: 4, such as, e.g., a BoNT/D isoform of SEQ ID NO: 4 or a BoNT/D subtype of SEQ ID NO: 4. In still another aspect of this embodiment, a BoNT/D comprises a non-naturally occurring BoNT/D variant, such as, e.g., a conservative BoNT/D variant, a non-conservative BoNT/D variant or an active BoNT/D fragment, or any combination thereof. In still another aspect of this embodiment, a BoNT/D comprises a non-naturally occurring BoNT/D variant of SEQ ID NO: 4, such as, e.g., a conservative BoNT/D variant of SEQ ID NO: 4, a non-conservative BoNT/D variant of SEQ ID NO: 4 or an active BoNT/D fragment of SEQ ID NO: 4, or any combination thereof. In yet another aspect of this embodiment, a BoNT/D comprises a BoNT/D enzymatic domain or an active fragment thereof, a BoNT/D translocation domain or an active fragment thereof, a BoNT/D binding domain or an active fragment thereof, or any combination thereof. In yet another aspect of this embodiment, a BoNT/D comprising a BoNT/D enzymatic domain of amino acids 1-445 from SEQ ID NO: 4 or an active fragment thereof, a BoNT/D translocation domain of amino acids 446-862 from SEQ ID NO: 4 or an active fragment thereof, a BoNT/D binding domain of amino acids 863-1276 from SEQ ID NO: 4 or an active fragment thereof, and any combination thereof.

In other aspects of this embodiment, a BoNT/D comprises a polypeptide having, e.g., at least 70% amino acid identity with SEQ ID NO: 4, at least 75% amino acid identity with the SEQ ID NO: 4, at least 80% amino acid identity with SEQ ID NO: 4, at least 85% amino acid identity with SEQ ID NO: 4, at least 90% amino acid identity with SEQ ID NO: 4 or at least 95% amino acid identity with SEQ ID NO: 4. In yet other aspects of this embodiment, a BoNT/D comprises a polypeptide having, e.g., at most 70% amino acid identity with SEQ ID NO: 4, at most 75% amino acid identity with the SEQ ID NO: 4, at most 80% amino acid identity with SEQ ID NO: 4, at most 85% amino acid identity with SEQ ID NO: 4, at most 90% amino acid identity with SEQ ID NO: 4 or at most 95% amino acid identity with SEQ ID NO: 4.

In other aspects of this embodiment, a BoNT/D comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 4. In yet other aspects of this embodiment, a BoNT/D comprises a polypeptide having, e.g., at most one, two, three, four, five, six, seven, eight, nine, 10, 20, 30, 40, 50, 100, 200 or 500 non-contiguous amino acid deletions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 non-contiguous amino acid deletions relative to SEQ ID NO: 4. In still other aspects of this embodiment, a BoNT/D comprises a polypeptide having, e.g., at most one, two, three, four, five, six, seven, eight, nine, 10, 20, 30, 40, 50, 100, 200 or 500 non-contiguous amino acid additions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 non-contiguous amino acid additions relative to SEQ ID NO: 4.

In other aspects of this embodiment, a BoNT/D comprises a polypeptide having, e.g., at most one, two, three, four, five, six, seven, eight, nine, 10, 20, 30, 40, 50, 100, 200 or 500 contiguous amino acid substitutions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 contiguous amino acid substitutions relative to SEQ ID NO: 4. In yet other aspects of this embodiment, a BoNT/D comprises a polypeptide having, e.g., at most one, two, three, four, five, six, seven, eight, nine, 10, 20, 30, 40, 50, 100, 200 or 500 contiguous amino acid deletions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 contiguous amino acid deletions relative to SEQ ID NO: 4. In still other aspects of this embodiment, a BoNT/D comprises a polypeptide having, e.g., at most one, two, three, four, five, six, seven, eight, nine, 10, 20, 30, 40, 50, 100, 200 or 500 contiguous amino acid additions relative to SEQ ID NO: 4. In other aspects of this embodiment, a BoNT/D comprises a polypeptide having, e.g., at least one, two, three, four, five, six, seven, eight, nine, 10, 20, 30, 40, 50, 100, 200 or 500 contiguous amino acid additions relative to SEQ ID NO: 4.

In another embodiment, a Clostridial toxin comprises a BoNT/E. In an aspect of this embodiment, a BoNT/E comprises a BoNT/E enzymatic domain, a BoNT/E translocation domain and a BoNT/E binding domain. In another aspect of this embodiment, a BoNT/E comprises SEQ ID NO: 5. In another aspect of this embodiment, a BoNT/E comprises a naturally occurring BoNT/E variant, such as, e.g., a BoNT/E isoform or a BoNT/E subtype. In another aspect of this embodiment, a BoNT/E comprises a naturally occurring BoNT/E variant of SEQ ID NO: 5, such as, e.g., a BoNT/E isoform of SEQ ID NO: 5 or a BoNT/E subtype of SEQ ID NO: 5. In still another aspect of this embodiment, a BoNT/E comprises a non-naturally occurring BoNT/E variant, such as, e.g., a conservative BoNT/E variant, a non-conservative BoNT/E variant or an active BoNT/E fragment, or any combination thereof. In still another aspect of this embodiment, a BoNT/E comprises a non-naturally occurring BoNT/E variant of SEQ ID NO: 5, such as, e.g., a conservative BoNT/E variant of SEQ ID NO: 5, a non-conservative BoNT/E variant of SEQ ID NO: 5 or an active BoNT/E fragment of SEQ ID NO: 5, or any combination thereof. In yet another aspect of this embodiment, a BoNT/E comprising a BoNT/E enzymatic domain or an active fragment thereof, a BoNT/E translocation domain or active fragment thereof, a BoNT/E binding domain or active fragment thereof, and any combination thereof. In yet another aspect of this embodiment, a BoNT/E comprising a BoNT/E enzymatic domain of amino acids 1-422 from SEQ ID NO: 5 or active fragment thereof, a BoNT/E translocation domain of amino acids 423-845 from SEQ ID NO: 5 or active fragment thereof, a BoNT/E binding domain of amino acids 846-1252 from SEQ ID NO: 5 or active fragment thereof, and any combination thereof.

In other aspects of this embodiment, a BoNT/E comprises a polypeptide having, e.g., at least 70% amino acid identity with SEQ ID NO: 5, at least 75% amino acid identity with the SEQ ID NO: 5, at least 80% amino acid identity with SEQ ID NO: 5, at least 85% amino acid identity with SEQ ID NO: 5, at least 90% amino acid identity with SEQ ID NO: 5 or at least 95% amino acid identity with SEQ ID NO: 5. In yet other aspects of this embodiment, a BoNT/E comprises a polypeptide having, e.g., at most 70% amino acid identity with SEQ ID NO: 5, at most 75% amino acid identity with the SEQ ID NO: 5, at most 80% amino acid identity with SEQ ID NO: 5, at most 85% amino acid identity with SEQ ID NO: 5, at most 90% amino acid identity with SEQ ID NO: 5 or at most 95% amino acid identity with SEQ ID NO: 5.

In other aspects of this embodiment, a BoNT/E comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 5. In yet other aspects of this embodiment, a BoNT/E comprises a polypeptide having, e.g., at most one, two, three, four, five, six, seven, eight, nine, 10, 20, 30, 40, 50, 100, 200 or 500 non-contiguous amino acid deletions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 non-contiguous amino acid deletions relative to SEQ ID NO: 5. In still other aspects of this embodiment, a BoNT/E comprises a polypeptide having, e.g., at most one, two, three, four, five, six, seven, eight, nine, 10, 20, 30, 40, 50, 100, 200 or 500 non-contiguous amino acid additions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 non-contiguous amino acid additions relative to SEQ ID NO: 5.

In other aspects of this embodiment, a BoNT/E comprises a polypeptide having, e.g., at most one, two, three, four, five, six, seven, eight, nine, 10, 20, 30, 40, 50, 100, 200 or 500 contiguous amino acid substitutions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 contiguous amino acid substitutions relative to SEQ ID NO: 5. In yet other aspects of this embodiment, a BoNT/E comprises a polypeptide having, e.g., at most one, two, three, four, five, six, seven, eight, nine, 10, 20, 30, 40, 50, 100, 200 or 500 contiguous amino acid deletions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 contiguous amino acid deletions relative to SEQ ID NO: 5. In still other aspects of this embodiment, a BoNT/E comprises a polypeptide having, e.g., at most one, two, three, four, five, six, seven, eight, nine, 10, 20, 30, 40, 50, 100, 200 or 500 contiguous amino acid additions relative to SEQ ID NO: 5. In other aspects of this embodiment, a BoNT/E comprises a polypeptide having, e.g., at least one, two, three, four, five, six, seven, eight, nine, 10, 20, 30, 40, 50, 100, 200 or 500 contiguous amino acid additions relative to SEQ ID NO: 5.

In another embodiment, a Clostridial toxin comprises a BoNT/F. In an aspect of this embodiment, a BoNT/F comprises a BoNT/F enzymatic domain, a BoNT/F translocation domain and a BoNT/F binding domain. In another aspect of this embodiment, a BoNT/F comprises SEQ ID NO: 6. In another aspect of this embodiment, a BoNT/F comprises a naturally occurring BoNT/F variant, such as, e.g., a BoNT/F isoform or a BoNT/F subtype. In another aspect of this embodiment, a BoNT/F comprises a naturally occurring BoNT/F variant of SEQ ID NO: 6, such as, e.g., a BoNT/F isoform of SEQ ID NO: 6 or a BoNT/F subtype of SEQ ID NO: 6. In still another aspect of this embodiment, a BoNT/F comprises a non-naturally occurring BoNT/F variant, such as, e.g., a conservative BoNT/F variant, a non-conservative BoNT/F variant or an active BoNT/F fragment, or any combination thereof. In still another aspect of this embodiment, a BoNT/F comprises a non-naturally occurring BoNT/F variant of SEQ ID NO: 6, such as, e.g., a conservative BoNT/F variant of SEQ ID NO: 6, a non-conservative BoNT/F variant of SEQ ID NO: 6 or an active BoNT/F fragment of SEQ ID NO: 6, or any combination thereof. In yet another aspect of this embodiment, a BoNT/F comprises a BoNT/F enzymatic domain or active fragment thereof, a BoNT/F translocation domain or active fragment thereof, a BoNT/F binding domain or active fragment thereof, and any combination thereof. In yet another aspect of this embodiment, a BoNT/F comprises a BoNT/F enzymatic domain of amino acid 1-439 from SEQ ID NO: 6 or active fragment thereof, a BoNT/F translocation domain of amino acids 440-864 from SEQ ID NO: 6 or active fragment thereof, a BoNT/F binding domain of amino acids 865-1274 from SEQ ID NO: 6 or active fragment thereof, and any combination thereof.

In other aspects of this embodiment, a BoNT/F comprises a polypeptide having, e.g., at least 70% amino acid identity with SEQ ID NO: 6, at least 75% amino acid identity with the SEQ ID NO: 6, at least 80% amino acid identity with SEQ ID NO: 6, at least 85% amino acid identity with SEQ ID NO: 6, at least 90% amino acid identity with SEQ ID NO: 6 or at least 95% amino acid identity with SEQ ID NO: 6. In yet other aspects of this embodiment, a BoNT/F comprises a polypeptide having, e.g., at most 70% amino acid identity with SEQ ID NO: 6, at most 75% amino acid identity with the SEQ ID NO: 6, at most 80% amino acid identity with SEQ ID NO: 6, at most 85% amino acid identity with SEQ ID NO: 6, at most 90% amino acid identity with SEQ ID NO: 6 or at most 95% amino acid identity with SEQ ID NO: 6.

In other aspects of this embodiment, a BoNT/F comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 6. In yet other aspects of this embodiment, a BoNT/F comprises a polypeptide having, e.g., at most one, two, three, four, five, six, seven, eight, nine, 10, 20, 30, 40, 50, 100, 200 or 500 non-contiguous amino acid deletions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 non-contiguous amino acid deletions relative to SEQ ID NO: 6. In still other aspects of this embodiment, a BoNT/F comprises a polypeptide having, e.g., at most one, two, three, four, five, six, seven, eight, nine, 10, 20, 30, 40, 50, 100, 200 or 500 non-contiguous amino acid additions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 non-contiguous amino acid additions relative to SEQ ID NO: 6.

In other aspects of this embodiment, a BoNT/F comprises a polypeptide having, e.g., at most one, two, three, four, five, six, seven, eight, nine, 10, 20, 30, 40, 50, 100, 200 or 500 contiguous amino acid substitutions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 contiguous amino acid substitutions relative to SEQ ID NO: 6. In yet other aspects of this embodiment, a BoNT/F comprises a polypeptide having, e.g., at most one, two, three, four, five, six, seven, eight, nine, 10, 20, 30, 40, 50, 100, 200 or 500 contiguous amino acid deletions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 contiguous amino acid deletions relative to SEQ ID NO: 6. In still other aspects of this embodiment, a BoNT/F comprises a polypeptide having, e.g., at most one, two, three, four, five, six, seven, eight, nine, 10, 20, 30, 40, 50, 100, 200 or 500 contiguous amino acid additions relative to SEQ ID NO: 6. In other aspects of this embodiment, a BoNT/F comprises a polypeptide having, e.g., at least one, two, three, four, five, six, seven, eight, nine, 10, 20, 30, 40, 50, 100, 200 or 500 contiguous amino acid additions relative to SEQ ID NO: 6.

In another embodiment, a Clostridial toxin comprises a BoNT/G. In an aspect of this embodiment, a BoNT/G comprises a BoNT/G enzymatic domain, a BoNT/G translocation domain and a BoNT/G binding domain. In another aspect of this embodiment, a BoNT/G comprises SEQ ID NO: 7. In another aspect of this embodiment, a BoNT/G comprises a naturally occurring BoNT/G variant, such as, e.g., a BoNT/G isoform or a BoNT/G subtype. In another aspect of this embodiment, a BoNT/G comprises a naturally occurring BoNT/G variant of SEQ ID NO: 7, such as, e.g., a BoNT/G isoform of SEQ ID NO: 7 or a BoNT/G subtype of SEQ ID NO: 7. In still another aspect of this embodiment, a BoNT/G comprises a non-naturally occurring BoNT/G variant, such as, e.g., a conservative BoNT/G variant, a non-conservative BoNT/G variant or an active BoNT/G fragment, or any combination thereof. In still another aspect of this embodiment, a BoNT/D comprises a non-naturally occurring BoNT/G variant of SEQ ID NO: 7, such as, e.g., a conservative BoNT/G variant of SEQ ID NO: 7, a non-conservative BoNT/G variant of SEQ ID NO: 7 or an active BoNT/G fragment of SEQ ID NO: 7, or any combination thereof. In yet another aspect of this embodiment, a BoNT/G comprises a BoNT/G enzymatic domain or an active fragment thereof, a BoNT/G translocation domain or an active fragment thereof, a BoNT/G binding domain or an active fragment thereof, or any combination thereof. In yet another aspect of this embodiment, a BoNT/G comprising a BoNT/G enzymatic domain of amino acids 1-446 from SEQ ID NO: 7 or an active fragment thereof, a BoNT/G translocation domain of amino acids 447-863 from SEQ ID NO: 7 or an active fragment thereof, a BoNT/G binding domain of amino acids 864-1297 from SEQ ID NO: 7 or an active fragment thereof, and any combination thereof.

In other aspects of this embodiment, a BoNT/G comprises a polypeptide having, e.g., at least 70% amino acid identity with SEQ ID NO: 7, at least 75% amino acid identity with the SEQ ID NO: 7, at least 80% amino acid identity with SEQ ID NO: 7, at least 85% amino acid identity with SEQ ID NO: 7, at least 90% amino acid identity with SEQ ID NO: 7 or at least 95% amino acid identity with SEQ ID NO: 7. In yet other aspects of this embodiment, a BoNT/G comprises a polypeptide having, e.g., at most 70% amino acid identity with SEQ ID NO: 7, at most 75% amino acid identity with the SEQ ID NO: 7, at most 80% amino acid identity with SEQ ID NO: 7, at most 85% amino acid identity with SEQ ID NO: 7, at most 90% amino acid identity with SEQ ID NO: 7 or at most 95% amino acid identity with SEQ ID NO: 7.

In other aspects of this embodiment, a BoNT/G comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 7. In yet other aspects of this embodiment, a BoNT/G comprises a polypeptide having, e.g., at most one, two, three, four, five, six, seven, eight, nine, 10, 20, 30, 40, 50, 100, 200 or 500 non-contiguous amino acid deletions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 non-contiguous amino acid deletions relative to SEQ ID NO: 7. In still other aspects of this embodiment, a BoNT/G comprises a polypeptide having, e.g., at most one, two, three, four, five, six, seven, eight, nine, 10, 20, 30, 40, 50, 100, 200 or 500 non-contiguous amino acid additions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 non-contiguous amino acid additions relative to SEQ ID NO: 7.

In other aspects of this embodiment, a BoNT/G comprises a polypeptide having, e.g., at most one, two, three, four, five, six, seven, eight, nine, 10, 20, 30, 40, 50, 100, 200 or 500 contiguous amino acid substitutions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 contiguous amino acid substitutions relative to SEQ ID NO: 7. In yet other aspects of this embodiment, a BoNT/G comprises a polypeptide having, e.g., at most one, two, three, four, five, six, seven, eight, nine, 10, 20, 30, 40, 50, 100, 200 or 500 contiguous amino acid deletions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 contiguous amino acid deletions relative to SEQ ID NO: 7. In still other aspects of this embodiment, a BoNT/G comprises a polypeptide having, e.g., at most one, two, three, four, five, six, seven, eight, nine, 10, 20, 30, 40, 50, 100, 200 or 500 contiguous amino acid additions relative to SEQ ID NO: 7. In other aspects of this embodiment, a BoNT/G comprises a polypeptide having, e.g., at least one, two, three, four, five, six, seven, eight, nine, 10, 20, 30, 40, 50, 100, 200 or 500 contiguous amino acid additions relative to SEQ ID NO: 7.

In another embodiment, a Clostridial toxin comprises a TeNT. In an aspect of this embodiment, a TeNT comprises a TeNT enzymatic domain, a TeNT translocation domain and a TeNT binding domain. In an aspect of this embodiment, a TeNT comprises SEQ ID NO: 8. In another aspect of this embodiment, a TeNT comprises a naturally occurring TeNT variant, such as, e.g., a TeNT isoform or a TeNT subtype. In another aspect of this embodiment, a TeNT comprises a naturally occurring TeNT variant of SEQ ID NO: 8, such as, e.g., a TeNT isoform of SEQ ID NO: 8 or a TeNT subtype of SEQ ID NO: 8. In still another aspect of this embodiment, a TeNT comprises a non-naturally occurring TeNT variant, such as, e.g., a conservative TeNT variant, a non-conservative TeNT variant or an active TeNT fragment, or any combination thereof. In still another aspect of this embodiment, a TeNT comprises a non-naturally occurring TeNT variant of SEQ ID NO: 8, such as, e.g., a conservative TeNT variant of SEQ ID NO: 8, a non-conservative TeNT variant of SEQ ID NO: 8 or an active TeNT fragment of SEQ ID NO: 8, or any combination thereof. In yet another aspect of this embodiment, a TeNT comprising a TeNT enzymatic domain or an active fragment thereof, a TeNT translocation domain or active fragment thereof, a TeNT binding domain or active fragment thereof, and any combination thereof. In yet another aspect of this embodiment, a TeNT comprising a TeNT enzymatic domain of amino acids 1-457 from SEQ ID NO: 8 or active fragment thereof, a TeNT translocation domain of amino acids 458-879 from SEQ ID NO: 8 or active fragment thereof, a TeNT binding domain of amino acids 880-1315 from SEQ ID NO: 8 or active fragment thereof, and any combination thereof.

In other aspects of this embodiment, a TeNT comprises a polypeptide having, e.g., at least 70% amino acid identity with SEQ ID NO: 8, at least 75% amino acid identity with the SEQ ID NO: 8, at least 80% amino acid identity with SEQ ID NO: 8, at least 85% amino acid identity with SEQ ID NO: 8, at least 90% amino acid identity with SEQ ID NO: 8 or at least 95% amino acid identity with SEQ ID NO: 8. In yet other aspects of this embodiment, a TeNT comprises a polypeptide having, e.g., at most 70% amino acid identity with SEQ ID NO: 8, at most 75% amino acid identity with the SEQ ID NO: 8, at most 80% amino acid identity with SEQ ID NO: 8, at most 85% amino acid identity with SEQ ID NO: 8, at most 90% amino acid identity with SEQ ID NO: 8 or at most 95% amino acid identity with SEQ ID NO: 8.

In other aspects of this embodiment, a TeNT comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 8. In yet other aspects of this embodiment, a TeNT comprises a polypeptide having, e.g., at most one, two, three, four, five, six, seven, eight, nine, 10, 20, 30, 40, 50, 100, 200 or 500 non-contiguous amino acid deletions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 non-contiguous amino acid deletions relative to SEQ ID NO: 8. In still other aspects of this embodiment, a TeNT comprises a polypeptide having, e.g., at most one, two, three, four, five, six, seven, eight, nine, 10, 20, 30, 40, 50, 100, 200 or 500 non-contiguous amino acid additions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 non-contiguous amino acid additions relative to SEQ ID NO: 8.

In other aspects of this embodiment, a TeNT comprises a polypeptide having, e.g., at most one, two, three, four, five, six, seven, eight, nine, 10, 20, 30, 40, 50, 100, 200 or 500 contiguous amino acid substitutions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 contiguous amino acid substitutions relative to SEQ ID NO: 8. In yet other aspects of this embodiment, a TeNT comprises a polypeptide having, e.g., at most one, two, three, four, five, six, seven, eight, nine, 10, 20, 30, 40, 50, 100, 200 or 500 contiguous amino acid deletions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 contiguous amino acid deletions relative to SEQ ID NO: 8. In still other aspects of this embodiment, a TeNT comprises a polypeptide having, e.g., at most one, two, three, four, five, six, seven, eight, nine, 10, 20, 30, 40, 50, 100, 200 or 500 contiguous amino acid additions relative to SEQ ID NO: 8. In other aspects of this embodiment, a TeNT comprises a polypeptide having, e.g., at least one, two, three, four, five, six, seven, eight, nine, 10, 20, 30, 40, 50, 100, 200 or 500 contiguous amino acid additions relative to SEQ ID NO: 8.

In another embodiment, a Clostridial toxin comprises a BaNT. In an aspect of this embodiment, a BaNT comprises a BaNT enzymatic domain, a BaNT translocation domain and a BaNT binding domain. In another aspect of this embodiment, a BaNT comprises SEQ ID NO: 9. In another aspect of this embodiment, a BaNT comprises a naturally occurring BaNT variant, such as, e.g., a BaNT isoform or a BaNT subtype. In another aspect of this embodiment, a BaNT comprises a naturally occurring BaNT variant of SEQ ID NO: 9, such as, e.g., a BaNT isoform of SEQ ID NO: 9 or a BaNT subtype of SEQ ID NO: 9. In still another aspect of this embodiment, a BaNT comprises a non-naturally occurring BaNT variant, such as, e.g., a conservative BaNT variant, a non-conservative BaNT variant or an active BaNT fragment, or any combination thereof. In still another aspect of this embodiment, a BaNT comprises a non-naturally occurring BaNT variant of SEQ ID NO: 9, such as, e.g., a conservative BaNT variant of SEQ ID NO: 9, a non-conservative BaNT variant of SEQ ID NO: 9 or an active BaNT fragment of SEQ ID NO: 9, or any combination thereof. In yet another aspect of this embodiment, a BaNT comprises a BaNT enzymatic domain or an active fragment thereof, a BaNT translocation domain or an active fragment thereof, a BaNT binding domain or an active fragment thereof, or any combination thereof. In yet another aspect of this embodiment, a BaNT comprising a BaNT enzymatic domain of amino acids 1-448 from SEQ ID NO: 9 or an active fragment thereof, a BaNT translocation domain of amino acids 449-871 from SEQ ID NO: 9 or an active fragment thereof, a BaNT binding domain of amino acids 872-1296 from SEQ ID NO: 9 or an active fragment thereof, and any combination thereof.

In other aspects of this embodiment, a BaNT comprises a polypeptide having, e.g., at least 70% amino acid identity with SEQ ID NO: 9, at least 75% amino acid identity with the SEQ ID NO: 9, at least 80% amino acid identity with SEQ ID NO: 9, at least 85% amino acid identity with SEQ ID NO: 9, at least 90% amino acid identity with SEQ ID NO: 9 or at least 95% amino acid identity with SEQ ID NO: 9. In yet other aspects of this embodiment, a BaNT comprises a polypeptide having, e.g., at most 70% amino acid identity with SEQ ID NO: 9, at most 75% amino acid identity with the SEQ ID NO: 9, at most 80% amino acid identity with SEQ ID NO: 9, at most 85% amino acid identity with SEQ ID NO: 9, at most 90% amino acid identity with SEQ ID NO: 9 or at most 95% amino acid identity with SEQ ID NO: 9.

In other aspects of this embodiment, a BaNT comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 9. In yet other aspects of this embodiment, a BaNT comprises a polypeptide having, e.g., at most one, two, three, four, five, six, seven, eight, nine, 10, 20, 30, 40, 50, 100, 200 or 500 non-contiguous amino acid deletions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 non-contiguous amino acid deletions relative to SEQ ID NO: 9. In still other aspects of this embodiment, a BaNT comprises a polypeptide having, e.g., at most one, two, three, four, five, six, seven, eight, nine, 10, 20, 30, 40, 50, 100, 200 or 500 non-contiguous amino acid additions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 non-contiguous amino acid additions relative to SEQ ID NO: 9.

In other aspects of this embodiment, a BaNT comprises a polypeptide having, e.g., at most one, two, three, four, five, six, seven, eight, nine, 10, 20, 30, 40, 50, 100, 200 or 500 contiguous amino acid substitutions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 contiguous amino acid substitutions relative to SEQ ID NO: 9. In yet other aspects of this embodiment, a BaNT comprises a polypeptide having, e.g., at most one, two, three, four, five, six, seven, eight, nine, 10, 20, 30, 40, 50, 100, 200 or 500 contiguous amino acid deletions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 contiguous amino acid deletions relative to SEQ ID NO: 9. In still other aspects of this embodiment, a BaNT comprises a polypeptide having, e.g., at most one, two, three, four, five, six, seven, eight, nine, 10, 20, 30, 40, 50, 100, 200 or 500 contiguous amino acid additions relative to SEQ ID NO: 9. In other aspects of this embodiment, a BaNT comprises a polypeptide having, e.g., at least one, two, three, four, five, six, seven, eight, nine, 10, 20, 30, 40, 50, 100, 200 or 500 contiguous amino acid additions relative to SEQ ID NO: 9.

In another embodiment, a Clostridial toxin comprises a BuNT. In an aspect of this embodiment, a BuNT comprises a BuNT enzymatic domain, a BuNT translocation domain and a BuNT binding domain.

In another aspect of this embodiment, a BuNT comprises SEQ ID NO: 10. In another aspect of this embodiment, a BuNT comprises a naturally occurring BuNT variant, such as, e.g., a BuNT isoform or a BuNT subtype. In another aspect of this embodiment, a BuNT comprises a naturally occurring BuNT variant of SEQ ID NO: 10, such as, e.g., a BuNT isoform of SEQ ID NO: 10 or a BuNT subtype of SEQ ID NO: 10. In still another aspect of this embodiment, a BuNT comprises a non-naturally occurring BuNT variant, such as, e.g., a conservative BuNT variant, a non-conservative BuNT variant or an active BuNT fragment, or any combination thereof. In still another aspect of this embodiment, a BuNT comprises a non-naturally occurring BuNT variant of SEQ ID NO: 10, such as, e.g., a conservative BuNT variant of SEQ ID NO: 10, a non-conservative BuNT variant of SEQ ID NO: 10 or an active BuNT fragment of SEQ ID NO: 10, or any combination thereof. In yet another aspect of this embodiment, a BuNT comprises a BuNT enzymatic domain or an active fragment thereof, a BuNT translocation domain or an active fragment thereof, a BuNT binding domain or an active fragment thereof, or any combination thereof. In yet another aspect of this embodiment, a BuNT comprising a BuNT enzymatic domain of amino acids 1-448 from SEQ ID NO: 10 or an active fragment thereof, a BuNT translocation domain of amino acids 449-871 from SEQ ID NO: 10 or an active fragment thereof, a BuNT binding domain of amino acids 872-1296 from SEQ ID NO: 10 or an active fragment thereof, and any combination thereof.

In other aspects of this embodiment, a BuNT comprises a polypeptide having, e.g., at least 70% amino acid identity with SEQ ID NO: 10, at least 75% amino acid identity with the SEQ ID NO: 10, at least 80% amino acid identity with SEQ ID NO: 10, at least 85% amino acid identity with SEQ ID NO: 10, at least 90% amino acid identity with SEQ ID NO: 10 or at least 95% amino acid identity with SEQ ID NO: 10. In yet other aspects of this embodiment, a BuNT comprises a polypeptide having, e.g., at most 70% amino acid identity with SEQ ID NO: 10, at most 75% amino acid identity with the SEQ ID NO: 10, at most 80% amino acid identity with SEQ ID NO: 10, at most 85% amino acid identity with SEQ ID NO: 10, at most 90% amino acid identity with SEQ ID NO: 10 or at most 95% amino acid identity with SEQ ID NO: 10.

In other aspects of this embodiment, a BuNT comprises a polypeptide having, e.g., at most one, two, three, four, five, six, seven, eight, nine, 10, 20, 30, 40, 50, 100, 200 or 500 non-contiguous amino acid substitutions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 non-contiguous amino acid substitutions relative to SEQ ID NO: 10. In yet other aspects of this embodiment, a BuNT comprises a polypeptide having, e.g., at most one, two, three, four, five, six, seven, eight, nine, 10, 20, 30, 40, 50, 100, 200 or 500 non-contiguous amino acid deletions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 non-contiguous amino acid deletions relative to SEQ ID NO: 10. In still other aspects of this embodiment, a BuNT comprises a polypeptide having, e.g., at most one, two, three, four, five, six, seven, eight, nine, 10, 20, 30, 40, 50, 100, 200 or 500 non-contiguous amino acid additions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 non-contiguous amino acid additions relative to SEQ ID NO: 10.

In other aspects of this embodiment, a BuNT comprises a polypeptide having, e.g., at most one, two, three, four, five, six, seven, eight, nine, 10, 20, 30, 40, 50, 100, 200 or 500 contiguous amino acid substitutions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 contiguous amino acid substitutions relative to SEQ ID NO: 10. In yet other aspects of this embodiment, a BuNT comprises a polypeptide having, e.g., at most one, two, three, four, five, six, seven, eight, nine, 10, 20, 30, 40, 50, 100, 200 or 500 contiguous amino acid deletions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 contiguous amino acid deletions relative to SEQ ID NO: 10. In still other aspects of this embodiment, a BuNT comprises a polypeptide having, e.g., at most one, two, three, four, five, six, seven, eight, nine, 10, 20, 30, 40, 50, 100, 200 or 500 contiguous amino acid additions relative to SEQ ID NO: 10. In other aspects of this embodiment, a BuNT comprises a polypeptide having, e.g., at least one, two, three, four, five, six, seven, eight, nine, 10, 20, 30, 40, 50, 100, 200 or 500 contiguous amino acid additions relative to SEQ ID NO: 10.

As mentioned above, a Clostridial toxin is converted from a single polypeptide form into a di-chain molecule by proteolytic cleavage. The location of the di-chain loop protease cleavage site for Clostridial toxins is shown (Table 2). Cleavage within the di-chain loop does not appear to be confined to a single peptide bond. Thus, cleavage of a Clostridial toxin with a naturally-occurring di-chain loop protease results in the lost of several residues centered around the original cleavage site. This loss is limited to a few amino acids located between the two cysteine residues that form the disulfide bridge. As a non-limiting example, BoNT/A single-chain polypeptide cleavage ultimately results in the loss of a ten amino acids within the di-chain loop. For BoNTs, cleavage at K448-A449 converts the single-chain form of BoNT/A into the di-chain form; cleavage at K441-A442 converts the single-chain form of BoNT/B into the di-chain form; cleavage at K449-T450 converts the single-chain form of BoNT/C1 into the di-chain form; cleavage at R445-D446 converts the single-chain form of BoNT/D into the di-chain form; cleavage at R422-K423 converts the single-chain form of BoNT/E into the di-chain form; cleavage at K439-A440 converts the single-chain form of BoNT/F into the di-chain form; and cleavage at K446-S447 converts the single-chain form of BoNT/G into the di-chain form. Proteolytic cleavage of the single-chain form of TeNT at of A457-S458 results in the di-chain form. Proteolytic cleavage of the single-chain form of BaNT at of K431-N432 results in the di-chain form. Proteolytic cleavage of the single-chain form of BuNT at of R422-K423 results in the di-chain form.

TABLE 2
Di-chain Loop Region of Clostridial Toxins

| | | Di-Chain Loop Region Including a |
|---|---|---|
| Toxin | SEQ ID NO: | Di-Chain Protease Cleavage Site |
| BoNT/A | 11 | CVRGIITSKTKSLDKGYNK*----ALNDLC |
| BoNT/B | 12 | CKSVK*-------------------APGIC |
| BoNT/C1 | 13 | CHKAIDGRSLYNK*------------TLDC |
| BoNT/D | 14 | CLRLTKNSR*---------------DDSTC |
| BoNT/E | 15 | CKNIVSVKGIR*--------------KSIC |
| BoNT/F | 16 | CKSVIPRKGTK*------------APPRLC |
| BoNT/G | 17 | CKPVMYKNTGK*--------------SEQC |
| TeNT | 18 | CKKIIPPTNIRENLYNRTA*SLTDLGGELC |
| BaNT | 19 | CKSIVSKKGTK*--------------NSLC |
| BuNT | 20 | CKNIVSVKGIR*--------------KSIC |

The amino acid sequence displayed are as follows: BoNT/A, residues 430-454 of SEQ ID NO: 1; BoNT/B, residues 437-446 of SEQ ID NO: 2; BoNT/C1, residues 437-453 of SEQ ID NO: 3; BoNT/D, residues 437-450 of SEQ ID NO: 4; BoNT/E, residues 412-426 of SEQ ID NO: 5; BoNT/F, residues 429-445 of SEQ ID NO: 6; BoNT/G, residues 436-450 of SEQ ID NO: 7; TeNT, residues 439-467 of SEQ ID NO: 8; BaNT, residues 421-435 of SEQ ID NO: 9; and BuNT, residues 412-426 of SEQ ID NO: 10. An asterisks (*) indicates the peptide bond of the P₁—P_{1′} cleavage site that is believed to be cleaved by a Clostridial toxin di-chain loop protease.

However, it should also be noted that additional cleavage sites within the di-chain loop also appear to be cleaved resulting in the generation of a small peptide fragment being lost. As a non-limiting example, BoNT/A single-chain polypeptide cleavage ultimately results in the loss of a ten amino acid fragment within the di-chain loop. Thus, cleavage at S441-L442 converts the single polypeptide form of BoNT/A into the di-chain form; cleavage at G444-I445 converts the single polypeptide form of BoNT/B into the di-chain form; cleavage at S445-L446 converts the single polypeptide form of BoNT/C1 into the di-chain form; cleavage at K442-N443 converts the single polypeptide form of BoNT/D into the di-chain form; cleavage at K419-G420 converts the single polypeptide form of BoNT/E into the di-chain form; cleavage at K423-S424 converts the single polypeptide form of BoNT/E into the di-chain form; cleavage at K436-G437 converts the single polypeptide form of BoNT/F into the di-chain form; cleavage at T444-G445 converts the single polypeptide form of BoNT/G into the di-chain form; and cleavage at E448-Q449 converts the single polypeptide form of BoNT/G into the di-chain form.

Aspects of the present invention provide, in part, a Clostridial toxin di-chain loop region. As used herein, the term “Clostridial toxin di-chain loop region” means the loop region of a Clostridial toxin formed by a disulfide bridge located between the LC domain and the HC domain of a naturally-occurring Clostridial toxin. A Clostridial toxin di-chain loop region includes, without limitation, a BoNT/A di-chain loop region, a BoNT/B di-chain loop region, a BoNT/C1 di-chain loop region, a BoNT/D di-chain loop region, a BoNT/E di-chain loop region, a BoNT/F di-chain loop region, a BoNT/G di-chain loop region, a TeNT di-chain loop region, a BaNT di-chain loop region, and a BuNT di-chain loop region. A non-limiting example of a BoNT/A di-chain loop region is amino acid sequence CVRGIITSKTKSLDKGYNKALNDLC (SEQ ID NO: 11). A non-limiting example of a BoNT/B di-chain loop region is the amino acid sequence CKSVKAPGIC (SEQ ID NO: 12). A non-limiting example of a BoNT/C1 di-chain loop region is the amino acid sequence CHKAIDGRSLYNKTLDC (SEQ ID NO: 13). A non-limiting example of a BoNT/D di-chain loop region is the amino acid sequence CLRLTKNSRDDSTC (SEQ ID NO: 14). A non-limiting example of a BoNT/E di-chain loop region is the amino acid sequence CKNIVSVKGIRKSIC (SEQ ID NO: 15). A non-limiting example of a BoNT/F di-chain loop region is the amino acid sequence CKSVIPRKGTKAPPRLC (SEQ ID NO: 16). A non-limiting example of a BoNT/G di-chain loop region is the amino acid sequence CKPVMYKNTGKSEQC (SEQ ID NO: 17). A non-limiting example of a TeNT di-chain loop region is the amino acid sequence CKKIIPPTNIRENLYNRTASLTDLGGELC (SEQ ID NO: 18). A non-limiting example of a BaNT di-chain loop region is the amino acid sequence CKSIVSKKGTKNSLC (SEQ ID NO: 19). A non-limiting example of a BuNT di-chain loop region is the amino acid sequence CKNIVSVKGIRKSIC (SEQ ID NO: 20). As discussed below, SEQ ID NO: 11 through SEQ 1N NO: 20 can serve as reference Clostridial toxin di-chain loop region sequences.

A Clostridial toxin di-chain loop region useful in aspects of the invention includes, without limitation, naturally occurring Clostridial toxin di-chain loop region; naturally occurring Clostridial toxin di-chain loop region variants; and non-naturally-occurring Clostridial toxin di-chain loop region variants, such as, e.g., conservative Clostridial toxin di-chain loop region variants, non-conservative Clostridial toxin di-chain loop region variants and Clostridial toxin di-chain loop region peptidomimetics. As used herein, the term “Clostridial toxin di-chain loop region variant,” whether naturally-occurring or non-naturally-occurring, means a Clostridial toxin di-chain loop region that has at least one amino acid change from the corresponding region of the disclosed reference sequences and can be described in percent identity to the corresponding region of that reference sequence. Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

As used herein, the term “naturally occurring Clostridial toxin di-chain loop region variant” means any Clostridial toxin di-chain loop region produced without the aid of any human manipulation, including, without limitation, Clostridial toxin di-chain loop region isoforms produced from alternatively-spliced transcripts, Clostridial toxin di-chain loop region isoforms produced by spontaneous mutation and Clostridial toxin di-chain loop region subtypes. Non-limiting examples of a Clostridial toxin di-chain loop region isoform include, e.g., BoNT/A di-chain loop region isoforms, BoNT/B di-chain loop region isoforms, BoNT/C1 di-chain loop region isoforms, BoNT/D di-chain loop region isoforms, BoNT/E di-chain loop region isoforms, BoNT/F di-chain loop region isoforms, BoNT/G di-chain loop region isoforms, TeNT di-chain loop region isoforms, BaNT di-chain loop region isoforms, and BuNT di-chain loop region isoforms. Non-limiting examples of a Clostridial toxin subtype include, e.g., BoNT/A di-chain loop region subtypes such as, e.g., a BoNT/A1 di-chain loop region, a BoNT/A2 di-chain loop region, a BoNT/A3 di-chain loop region and a BoNT/A4 di-chain loop region; BoNT/B di-chain loop region subtypes, such as, e.g., a BoNT/B1 di-chain loop region, a BoNT/B2 di-chain loop region, a BoNT/B bivalent di-chain loop region and a BoNT/B nonproteolytic di-chain loop region; BoNT/C1 di-chain loop region subtypes, such as, e.g., a BoNT/C1-1 di-chain loop region and a BoNT/C1-2 di-chain loop region; BoNT/E di-chain loop region subtypes, such as, e.g., a BoNT/E1 di-chain loop region, a BoNT/E2 di-chain loop region and a BoNT/E3 di-chain loop region; and BoNT/F di-chain loop region subtypes, such as, e.g., a BoNT/F1 di-chain loop region, a BoNT/F2 di-chain loop region, a BoNT/F3 di-chain loop region and a BoNT/F4 di-chain loop region.

As used herein, the term “non-naturally occurring Clostridial toxin di-chain loop region variant” means any Clostridial toxin di-chain loop region produced with the aid of human manipulation, including, without limitation, Clostridial toxin di-chain loop region variants produced by genetic engineering using random mutagenesis or rational design and Clostridial toxin di-chain loop region variants produced by chemical synthesis. Non-limiting examples of non-naturally occurring Clostridial toxin di-chain loop region variants include, e.g., conservative Clostridial toxin di-chain loop region variants, non-conservative Clostridial toxin di-chain loop region variants and Clostridial toxin di-chain loop region peptidomimetics.

As used herein, the term “conservative Clostridial toxin di-chain loop region variant” means a Clostridial toxin di-chain loop region that has at least one amino acid substituted by another amino acid or an amino acid analog that has at least one property similar to that of the original amino acid from the reference Clostridial toxin di-chain loop region sequence. Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative Clostridial toxin di-chain loop region variant can function in substantially the same manner as the reference Clostridial toxin di-chain loop region on which the conservative Clostridial toxin di-chain loop region variant is based, and can be substituted for the reference Clostridial toxin di-chain loop region in any aspect of the present invention. A conservative Clostridial toxin di-chain loop region variant may substitute one or more amino acids, two or more amino acids, three or more amino acids, four or more amino acids or five or more amino acids from the reference Clostridial toxin di-chain loop region on which the conservative Clostridial toxin di-chain loop region variant is based. A conservative Clostridial toxin di-chain loop region variant can also possess at least 50% amino acid identity, 65% amino acid identity, 75% amino acid identity, 85% amino acid identity or 95% amino acid identity to the reference Clostridial toxin di-chain loop region on which the conservative Clostridial toxin di-chain loop region variant is based. Non-limiting examples of a conservative Clostridial toxin di-chain loop region variant include, e.g., conservative BoNT/A di-chain loop region variants, conservative BoNT/B di-chain loop region variants, conservative BoNT/C1 di-chain loop region variants, conservative BoNT/D di-chain loop region variants, conservative BoNT/E di-chain loop region variants, conservative BoNT/F di-chain loop region variants, conservative BoNT/G di-chain loop region variants, conservative TeNT di-chain loop region variants, conservative BaNT di-chain loop region variants and conservative BuNT di-chain loop region variants.

As used herein, the term “non-conservative Clostridial toxin di-chain loop region variant” means a Clostridial toxin di-chain loop region in which 1) at least one amino acid is deleted from the reference Clostridial toxin di-chain loop region on which the non-conservative Clostridial toxin di-chain loop region variant is based; 2) at least one amino acid added to the reference Clostridial toxin di-chain loop region on which the non-conservative Clostridial toxin di-chain loop region is based; or 3) at least one amino acid is substituted by another amino acid or an amino acid analog that does not share any property similar to that of the original amino acid from the reference Clostridial toxin di-chain loop region sequence. A non-conservative Clostridial toxin di-chain loop region variant can function in substantially the same manner as the reference Clostridial toxin di-chain loop region on which the non-conservative Clostridial toxin di-chain loop region is based, and can be substituted for the reference Clostridial toxin di-chain loop region in any aspect of the present invention. A non-conservative Clostridial toxin di-chain loop region variant can add one or more amino acids, two or more amino acids, three or more amino acids, four or more amino acids, five or more amino acids, and ten or more amino acids to the reference Clostridial toxin di-chain loop region on which the non-conservative Clostridial toxin di-chain loop region variant is based. A non-conservative Clostridial toxin di-chain loop region may substitute one or more amino acids, two or more amino acids, three or more amino acids, four or more amino acids or five or more amino acids from the reference Clostridial toxin di-chain loop region on which the non-conservative Clostridial toxin di-chain loop region variant is based. A non-conservative Clostridial toxin di-chain loop region variant can also possess at least 50% amino acid identity, 65% amino acid identity, 75% amino acid identity, 85% amino acid identity or 95% amino acid identity to the reference Clostridial toxin di-chain loop region on which the non-conservative Clostridial toxin di-chain loop region variant is based. Non-limiting examples of a non-conservative Clostridial toxin di-chain loop region variant include, e.g., non-conservative BoNT/A di-chain loop region variants, non-conservative BoNT/B di-chain loop region variants, non-conservative BoNT/C1 di-chain loop region variants, non-conservative BoNT/D di-chain loop region variants, non-conservative BoNT/E di-chain loop region variants, non-conservative BoNT/F di-chain loop region variants, non-conservative BoNT/G di-chain loop region variants, non-conservative TeNT di-chain loop region variants, non-conservative BaNT di-chain loop region variants and non-conservative BuNT di-chain loop region variants.

As used herein, the term “Clostridial toxin di-chain loop region peptidomimetic” means a Clostridial toxin di-chain loop region that has at least one amino acid substituted by a non-natural oligomer that has at least one property similar to that of the first amino acid. Examples of properties include, without limitation, topography of a peptide primary structural element, functionality of a peptide primary structural element, topology of a peptide secondary structural element, functionality of a peptide secondary structural element, of the like, or any combination thereof. A Clostridial toxin di-chain loop region peptidomimetic can function in substantially the same manner as the reference Clostridial toxin di-chain loop region on which the Clostridial toxin di-chain loop region peptidomimetic is based, and can be substituted for the reference Clostridial toxin di-chain loop region in any aspect of the present invention. A Clostridial toxin di-chain loop region peptidomimetic may substitute one or more amino acids, two or more amino acids, three or more amino acids, four or more amino acids or five or more amino acids from the reference Clostridial toxin di-chain loop region on which the Clostridial toxin di-chain loop region peptidomimetic is based. A Clostridial toxin di-chain loop region peptidomimetic can also possess at least 50% amino acid identity, at least 65% amino acid identity, at least 75% amino acid identity, at least 85% amino acid identity or at least 95% amino acid identity to the reference Clostridial toxin di-chain loop region on which the Clostridial toxin di-chain loop region peptidomimetic is based. For examples of peptidomimetic methods see, e.g., Amy S. Ripka & Daniel H. Rich, Peptidomimetic design, 2(4) CURR. OPIN. CHEM. BIOL. 441-452 (1998); and M. Angels Estiarte & Daniel H. Rich, Peptidomimetics for Drug Design, 803-861 (BURGER'S MEDICINAL CHEMISTRY AND DRUG DISCOVERY Vol. 1 PRINCIPLE AND PRACTICE, Donald J. Abraham ed., Wiley-Interscience, 6^{th} ed 2003). Non-limiting examples of a Clostridial toxin di-chain loop region peptidomimetic include, e.g., BoNT/A di-chain loop region peptidomimetics, BoNT/B di-chain loop region peptidomimetics, BoNT/C1 di-chain loop region peptidomimetics, BoNT/D di-chain loop region peptidomimetics, BoNT/E di-chain loop region peptidomimetics, BoNT/F di-chain loop region peptidomimetics, BoNT/G di-chain loop region peptidomimetics, TeNT di-chain loop region peptidomimetics, BaNT di-chain loop region peptidomimetics and BuNT di-chain loop region peptidomimetics.

Aspects of the present invention provide, in part, a Clostridial toxin di-chain loop protease cleavage site. As used herein, the term “Clostridial toxin di-chain loop protease cleavage site” means means a P₁-P₁ scissile bond located within a Clostridial toxin di-chain loop region, together with adjacent or non-adjacent recognition elements, or both, sufficient for detectable proteolysis at the scissile bond by a Clostridial toxin di-chain loop protease under conditions suitable for Clostridial toxin di-chain loop protease activity. A Clostridial toxin di-chain loop region includes, without limitation, a BoNT/A di-chain loop protease cleavage site, a BoNT/B di-chain loop protease cleavage site, a BoNT/C1 di-chain loop protease cleavage site, a BoNT/D di-chain loop protease cleavage site, a BoNT/E di-chain loop protease cleavage site, a BoNT/F di-chain loop protease cleavage site, a BoNT/G di-chain loop protease cleavage site, a TeNT di-chain loop protease cleavage site, a BaNT di-chain loop protease cleavage site, and a BuNT di-chain loop protease cleavage site. Non-limiting examples of a BoNT/A di-chain loop protease cleavage site include the S441-L442 scissile bond and the K448-A449 scissile bond. Non-limiting examples of a BoNT/B di-chain loop protease cleavage site include the K441-A442 scissile bond and the G444-I445 scissile bond. Non-limiting examples of a BoNT/C1 di-chain loop protease cleavage site include the S445-L446 scissile bond and the K449-T450 scissile bond. Non-limiting examples of a BoNT/D di-chain loop protease cleavage site include the K442-N443 scissile bond and the R445—D446 scissile bond. Non-limiting examples of a BoNT/E di-chain loop protease cleavage site include the K419-G420 scissile bond, the R422—K423 scissile bond, and the K423-S424 scissile bond. Non-limiting examples of a BoNT/F di-chain loop protease cleavage site include the K436-G437 scissile bond and the K439-A440 scissile bond. Non-limiting examples of a BoNT/G di-chain loop protease cleavage site include the T444-G445 scissile bond, the K446-S447 scissile bond, and the E448-Q449 scissile bond. A non-limiting example of a TeNT di-chain loop protease cleavage site is the A457-S458 scissile bond. A non-limiting example of a BaNT di-chain loop protease cleavage site is the K431-N432 scissile bond. A non-limiting example of a BuNT di-chain loop protease cleavage site is the R422—K423 scissile bond.

Thus, in an embodiment, a modified Clostridial toxin comprises a BoNT/A di-chain loop region. In an aspect of this embodiment, a modified Clostridial toxin comprises a BoNT/A di-chain loop region including a BoNT/A di-chain loop protease cleavage site. In another aspect of this embodiment, a modified Clostridial toxin comprises a BoNT/A di-chain loop region including a BoNT/A di-chain loop protease cleavage site comprising the S441-L442 scissile bond. In another aspect of this embodiment, a modified Clostridial toxin comprises a BoNT/A di-chain loop region including a BoNT/A di-chain loop protease cleavage site comprising the K448-A449 scissile bond. In yet another aspect of this embodiment, a modified Clostridial toxin comprises the BoNT/A di-chain loop region of SEQ ID NO: 11.

In another aspect of this embodiment, a modified Clostridial toxin comprises a naturally occurring BoNT/A di-chain loop region variant. In another aspect of this embodiment, a modified Clostridial toxin comprises a naturally occurring BoNT/A di-chain loop region variant, such as, e.g., a BoNT/A di-chain loop region isoform, or a BoNT/A di-chain loop region subtype. In another aspect of this embodiment, a modified Clostridial toxin comprises a naturally occurring BoNT/A di-chain loop region variant of SEQ ID NO: 11, such as, e.g., a BoNT/A di-chain loop region isoform of SEQ ID NO: 11; or a BoNT/A di-chain loop region subtype of SEQ ID NO: 11. In still another aspect of this embodiment, a modified Clostridial toxin comprises a non-naturally occurring BoNT/A di-chain loop region variant, such as, e.g., a conservative BoNT/A di-chain loop region variant, a non-conservative BoNT/A di-chain loop region variant or a BoNT/A di-chain loop region peptidomimetic, or any combination thereof. In still another aspect of this embodiment, a modified Clostridial toxin comprises a non-naturally occurring BoNT/A di-chain loop region variant of SEQ ID NO: 11, such as, e.g., a conservative BoNT/A di-chain loop region variant of SEQ ID NO: 11, a non-conservative BoNT/A di-chain loop region variant of SEQ ID NO: 11 or a BoNT/A di-chain loop region peptidomimetic of SEQ ID NO: 11, or any combination thereof.

In other aspects of this embodiment, a modified Clostridial toxin comprises a BoNT/A di-chain loop region having, e.g., at least 50% amino acid identity with SEQ ID NO: 11, at least 60% amino acid identity with the SEQ ID NO: 11, at least 70% amino acid identity with SEQ ID NO: 11, at least 80% amino acid identity with SEQ ID NO: 11, or at least 90% amino acid identity with SEQ ID NO: 11. In still other aspects of this embodiment, a modified Clostridial toxin comprises a BoNT/A di-chain loop region having, e.g., at most 50% amino acid identity with SEQ ID NO: 11, at most 60% amino acid identity with the SEQ ID NO: 11, at most 70% amino acid identity with SEQ ID NO: 11, at most 80% amino acid identity with SEQ ID NO: 11, or at most 90% amino acid identity with SEQ ID NO: 11.

In other aspects of this embodiment, a modified Clostridial toxin comprises a BoNT/A di-chain loop region having, e.g., at most one, two, three, four, five, six, seven, eight, nine or ten non-contiguous amino acid substitutions relative to SEQ ID NO: 11. In still other aspects of this embodiment, a modified Clostridial toxin comprises a BoNT/A di-chain loop region having, e.g., at least one, two, three, four, five, six, seven, eight, nine or ten non-contiguous amino acid substitutions relative to SEQ ID NO: 11. In yet other aspects of this embodiment, a modified Clostridial toxin comprises a BoNT/A di-chain loop region having, e.g., at most one, two, three, four, five, six, seven, eight, nine or ten non-contiguous amino acid additions relative to SEQ ID NO: 11. In yet other aspects of this embodiment, a modified Clostridial toxin comprises a BoNT/A di-chain loop region having, e.g., at least one, two, three, four, five, six, seven, eight, nine or ten non-contiguous amino acid additions relative to SEQ ID NO: 11. In still other aspects of this embodiment, a modified Clostridial toxin comprises a BoNT/A di-chain loop region having, e.g., at most one, two, three, four, five, six, seven, eight, nine or ten non-contiguous amino acid deletions relative to SEQ ID NO: 11. In still other aspects of this embodiment, a modified Clostridial toxin comprises a BoNT/A di-chain loop region having, e.g., at least one, two, three, four, five, six, seven, eight, nine or ten non-contiguous amino acid deletions relative to SEQ ID NO: 11.

In other aspects of this embodiment, a modified Clostridial toxin comprises a BoNT/A di-chain loop region having, e.g., at most one, two, three, four, five, six, seven, eight, nine or ten contiguous amino acid substitutions relative to SEQ ID NO: 11. In still other aspects of this embodiment, a modified Clostridial toxin comprises a BoNT/A di-chain loop region having, e.g., at least one, two, three, four, five, six, seven, eight, nine or ten contiguous amino acid substitutions relative to SEQ ID NO: 11. In yet other aspects of this embodiment, a modified Clostridial toxin comprises a BoNT/A di-chain loop region having, e.g., at most one, two, three, four, five, six, seven, eight, nine or ten contiguous amino acid additions relative to SEQ ID NO: 11. In yet other aspects of this embodiment, a modified Clostridial toxin comprises a BoNT/A di-chain loop region having, e.g., at least one, two, three, four, five, six, seven, eight, nine or ten contiguous amino acid additions relative to SEQ ID NO: 11. In still other aspects of this embodiment, a modified Clostridial toxin comprises a BoNT/A di-chain loop region having, e.g., at most one, two, three, four, five, six, seven, eight, nine or ten contiguous amino acid deletions relative to SEQ ID NO: 11. In still other aspects of this embodiment, a modified Clostridial toxin comprises a BoNT/A di-chain loop region having, e.g., at least one, two, three, four, five, six, seven, eight, nine or ten contiguous amino acid deletions relative to SEQ ID NO: 11.

In another embodiment, a modified Clostridial toxin comprises a BoNT/B di-chain loop region. In an aspect of this embodiment, a modified Clostridial toxin comprises a BoNT/B di-chain loop region including a BoNT/B di-chain loop protease cleavage site. In another aspect of this embodiment, a modified Clostridial toxin comprises a BoNT/B di-chain loop region including a BoNT/B di-chain loop protease cleavage site comprising the K441-A442 scissile bond. In another aspect of this embodiment, a modified Clostridial toxin comprises a BoNT/B di-chain loop region including a BoNT/B di-chain loop protease cleavage site comprising the G444-I445 scissile bond. In yet another aspect of this embodiment, a modified Clostridial toxin comprises the BoNT/B di-chain loop region of SEQ ID NO: 12.

In another aspect of this embodiment, a modified Clostridial toxin comprises a naturally occurring BoNT/B di-chain loop region variant. In another aspect of this embodiment, a modified Clostridial toxin comprises a naturally occurring BoNT/B di-chain loop region variant, such as, e.g., a BoNT/B di-chain loop region isoform, or a BoNT/B di-chain loop region subtype. In another aspect of this embodiment, a modified Clostridial toxin comprises a naturally occurring BoNT/B di-chain loop region variant of SEQ ID NO: 12, such as, e.g., a BoNT/B di-chain loop region isoform of SEQ ID NO: 12; or a BoNT/B di-chain loop region subtype of SEQ ID NO: 12. In still another aspect of this embodiment, a modified Clostridial toxin comprises a non-naturally occurring BoNT/B di-chain loop region variant, such as, e.g., a conservative BoNT/B di-chain loop region variant, a non-conservative BoNT/B di-chain loop region variant or a BoNT/B di-chain loop region peptidomimetic, or any combination thereof. In still another aspect of this embodiment, a modified Clostridial toxin comprises a non-naturally occurring BoNT/B di-chain loop region variant of SEQ ID NO: 12, such as, e.g., a conservative BoNT/B di-chain loop region variant of SEQ ID NO: 12, a non-conservative BoNT/B di-chain loop region variant of SEQ ID NO: 12 or a BoNT/B di-chain loop region peptidomimetic of SEQ ID NO: 12, or any combination thereof.

In other aspects of this embodiment, a modified Clostridial toxin comprises a BoNT/B di-chain loop region having, e.g., at least 50% amino acid identity with SEQ ID NO: 12, at least 60% amino acid identity with the SEQ ID NO: 12, at least 70% amino acid identity with SEQ ID NO: 12, at least 80% amino acid identity with SEQ ID NO: 12, or at least 90% amino acid identity with SEQ ID NO: 12. In still other aspects of this embodiment, a modified Clostridial toxin comprises a BoNT/B di-chain loop region having, e.g., at most 50% amino acid identity with SEQ ID NO: 12, at most 60% amino acid identity with the SEQ ID NO: 12, at most 70% amino acid identity with SEQ ID NO: 12, at most 80% amino acid identity with SEQ ID NO: 12, or at most 90% amino acid identity with SEQ ID NO: 12.

In other aspects of this embodiment, a modified Clostridial toxin comprises a BoNT/B di-chain loop region having, e.g., at most one, two, three, four, or five non-contiguous amino acid substitutions relative to SEQ ID NO: 12. In still other aspects of this embodiment, a modified Clostridial toxin comprises a BoNT/B di-chain loop region having, e.g., at least one, two, three, four, or five non-contiguous amino acid substitutions relative to SEQ ID NO: 12. In yet other aspects of this embodiment, a modified Clostridial toxin comprises a BoNT/B di-chain loop region having, e.g., at most one, two, three, four, or five non-contiguous amino acid additions relative to SEQ ID NO: 12. In yet other aspects of this embodiment, a modified Clostridial toxin comprises a BoNT/B di-chain loop region having, e.g., at least one, two, three, four, or five non-contiguous amino acid additions relative to SEQ ID NO: 12. In still other aspects of this embodiment, a modified Clostridial toxin comprises a BoNT/B di-chain loop region having, e.g., at most one, two, three, four, or five non-contiguous amino acid deletions relative to SEQ ID NO: 12. In still other aspects of this embodiment, a modified Clostridial toxin comprises a BoNT/B di-chain loop region having, e.g., at least one, two, three, four, or five non-contiguous amino acid deletions relative to SEQ ID NO: 12.

In other aspects of this embodiment, a modified Clostridial toxin comprises a BoNT/B di-chain loop region having, e.g., at most one, two, three, four, or five contiguous amino acid substitutions relative to SEQ ID NO: 12. In still other aspects of this embodiment, a modified Clostridial toxin comprises a BoNT/B di-chain loop region having, e.g., at least one, two, three, four, or five contiguous amino acid substitutions relative to SEQ ID NO: 12. In yet other aspects of this embodiment, a modified Clostridial toxin comprises a BoNT/B di-chain loop region having, e.g., at most one, two, three, four, or five contiguous amino acid additions relative to SEQ ID NO: 12. In yet other aspects of this embodiment, a modified Clostridial toxin comprises a BoNT/B di-chain loop region having, e.g., at least one, two, three, four, or five contiguous amino acid additions relative to SEQ ID NO: 12. In still other aspects of this embodiment, a modified Clostridial toxin comprises a BoNT/B di-chain loop region having, e.g., at most one, two, three, four, or five contiguous amino acid deletions relative to SEQ ID NO: 12. In still other aspects of this embodiment, a modified Clostridial toxin comprises a BoNT/B di-chain loop region having, e.g., at least one, two, three, four, or five contiguous amino acid deletions relative to SEQ ID NO: 12.

In another embodiment, a modified Clostridial toxin comprises a BoNT/C1 di-chain loop region. In an aspect of this embodiment, a modified Clostridial toxin comprises a BoNT/C1 di-chain loop region including a BoNT/C1 di-chain loop protease cleavage site. In another aspect of this embodiment, a modified Clostridial toxin comprises a BoNT/C1 di-chain loop region including a BoNT/C1 di-chain loop protease cleavage site comprising the 5445-L446 scissile bond. In another aspect of this embodiment, a modified Clostridial toxin comprises a BoNT/C1 di-chain loop region including a BoNT/C1 di-chain loop protease cleavage site comprising the K449-T450 scissile bond. In yet another aspect of this embodiment, a modified Clostridial toxin comprises the BoNT/C1 di-chain loop region of SEQ ID NO: 13.

In another aspect of this embodiment, a modified Clostridial toxin comprises a naturally occurring BoNT/C1 di-chain loop region variant. In another aspect of this embodiment, a modified Clostridial toxin comprises a naturally occurring BoNT/C1 di-chain loop region variant, such as, e.g., a BoNT/C1 di-chain loop region isoform, or a BoNT/C1 di-chain loop region subtype. In another aspect of this embodiment, a modified Clostridial toxin comprises a naturally occurring BoNT/C1 di-chain loop region variant of SEQ ID NO: 13, such as, e.g., a BoNT/C1 di-chain loop region isoform of SEQ ID NO: 13; or a BoNT/C1 di-chain loop region subtype of SEQ ID NO: 13. In still another aspect of this embodiment, a modified Clostridial toxin comprises a non-naturally occurring BoNT/C1 di-chain loop region variant, such as, e.g., a conservative BoNT/C1 di-chain loop region variant, a non-conservative BoNT/C1 di-chain loop region variant or a BoNT/C1 di-chain loop region peptidomimetic, or any combination thereof. In still another aspect of this embodiment, a modified Clostridial toxin comprises a non-naturally occurring BoNT/C1 di-chain loop region variant of SEQ ID NO: 13, such as, e.g., a conservative BoNT/C1 di-chain loop region variant of SEQ ID NO: 13, a non-conservative BoNT/C1 di-chain loop region variant of SEQ ID NO: 13 or a BoNT/C1 di-chain loop region peptidomimetic of SEQ ID NO: 13, or any combination thereof.

In other aspects of this embodiment, a modified Clostridial toxin comprises a BoNT/C1 di-chain loop region having, e.g., at least 50% amino acid identity with SEQ ID NO: 13, at least 60% amino acid identity with the SEQ ID NO: 13, at least 70% amino acid identity with SEQ ID NO: 13, at least 80% amino acid identity with SEQ ID NO: 13, or at least 90% amino acid identity with SEQ ID NO: 13. In still other aspects of this embodiment, a modified Clostridial toxin comprises a BoNT/C1 di-chain loop region having, e.g., at most 50% amino acid identity with SEQ ID NO: 13, at most 60% amino acid identity with the SEQ ID NO: 13, at most 70% amino acid identity with SEQ ID NO: 13, at most 80% amino acid identity with SEQ ID NO: 13, or at most 90% amino acid identity with SEQ ID NO: 13.

In other aspects of this embodiment, a modified Clostridial toxin comprises a BoNT/C1 di-chain loop region having, e.g., at most one, two, three, four, five, six, seven, eight, nine or ten non-contiguous amino acid substitutions relative to SEQ ID NO: 13. In still other aspects of this embodiment, a modified Clostridial toxin comprises a BoNT/C1 di-chain loop region having, e.g., at least one, two, three, four, five, six, seven, eight, nine or ten non-contiguous amino acid substitutions relative to SEQ ID NO: 13. In yet other aspects of this embodiment, a modified Clostridial toxin comprises a BoNT/C1 di-chain loop region having, e.g., at most one, two, three, four, five, six, seven, eight, nine or ten non-contiguous amino acid additions relative to SEQ ID NO: 13. In yet other aspects of this embodiment, a modified Clostridial toxin comprises a BoNT/C1 di-chain loop region having, e.g., at least one, two, three, four, five, six, seven, eight, nine or ten non-contiguous amino acid additions relative to SEQ ID NO: 13. In still other aspects of this embodiment, a modified Clostridial toxin comprises a BoNT/C1 di-chain loop region having, e.g., at most one, two, three, four, five, six, seven, eight, nine or ten non-contiguous amino acid deletions relative to SEQ ID NO: 13. In still other aspects of this embodiment, a modified Clostridial toxin comprises a BoNT/C1 di-chain loop region having, e.g., at least one, two, three, four, five, six, seven, eight, nine or ten non-contiguous amino acid deletions relative to SEQ ID NO: 13.

In other aspects of this embodiment, a modified Clostridial toxin comprises a BoNT/C1 di-chain loop region having, e.g., at most one, two, three, four, five, six, seven, eight, nine or ten contiguous amino acid substitutions relative to SEQ ID NO: 13. In still other aspects of this embodiment, a modified Clostridial toxin comprises a BoNT/C1 di-chain loop region having, e.g., at least one, two, three, four, five, six, seven, eight, nine or ten contiguous amino acid substitutions relative to SEQ ID NO: 13. In yet other aspects of this embodiment, a modified Clostridial toxin comprises a BoNT/C1 di-chain loop region having, e.g., at most one, two, three, four, five, six, seven, eight, nine or ten contiguous amino acid additions relative to SEQ ID NO: 13. In yet other aspects of this embodiment, a modified Clostridial toxin comprises a BoNT/C1 di-chain loop region having, e.g., at least one, two, three, four, five, six, seven, eight, nine or ten contiguous amino acid additions relative to SEQ ID NO: 13. In still other aspects of this embodiment, a modified Clostridial toxin comprises a BoNT/C1 di-chain loop region having, e.g., at most one, two, three, four, five, six, seven, eight, nine or ten contiguous amino acid deletions relative to SEQ ID NO: 13. In still other aspects of this embodiment, a modified Clostridial toxin comprises a BoNT/C1 di-chain loop region having, e.g., at least one, two, three, four, five, six, seven, eight, nine or ten contiguous amino acid deletions relative to SEQ ID NO: 13.

In another embodiment, a modified Clostridial toxin comprises a BoNT/D di-chain loop region. In an aspect of this embodiment, a modified Clostridial toxin comprises a BoNT/D di-chain loop region including a BoNT/D di-chain loop protease cleavage site. In another aspect of this embodiment, a modified Clostridial toxin comprises a BoNT/D di-chain loop region including a BoNT/D di-chain loop protease cleavage site comprising the K442-N443 scissile bond. In another aspect of this embodiment, a modified Clostridial toxin comprises a BoNT/D di-chain loop region including a BoNT/D di-chain loop protease cleavage site comprising the R445-D446 scissile bond. In yet another aspect of this embodiment, a modified Clostridial toxin comprises the BoNT/D di-chain loop region of SEQ ID NO: 14.

In another aspect of this embodiment, a modified Clostridial toxin comprises a naturally occurring BoNT/D di-chain loop region variant. In another aspect of this embodiment, a modified Clostridial toxin comprises a naturally occurring BoNT/D di-chain loop region variant, such as, e.g., a BoNT/D di-chain loop region isoform, or a BoNT/D di-chain loop region subtype. In another aspect of this embodiment, a modified Clostridial toxin comprises a naturally occurring BoNT/D di-chain loop region variant of SEQ ID NO: 14, such as, e.g., a BoNT/D di-chain loop region isoform of SEQ ID NO: 14; or a BoNT/D di-chain loop region subtype of SEQ ID NO: 14. In still another aspect of this embodiment, a modified Clostridial toxin comprises a non-naturally occurring BoNT/D di-chain loop region variant, such as, e.g., a conservative BoNT/D di-chain loop region variant, a non-conservative BoNT/D di-chain loop region variant or a BoNT/D di-chain loop region peptidomimetic, or any combination thereof. In still another aspect of this embodiment, a modified Clostridial toxin comprises a non-naturally occurring BoNT/D di-chain loop region variant of SEQ ID NO: 14, such as, e.g., a conservative BoNT/D di-chain loop region variant of SEQ ID NO: 14, a non-conservative BoNT/D di-chain loop region variant of SEQ ID NO: 14 or a BoNT/D di-chain loop region peptidomimetic of SEQ ID NO: 14, or any combination thereof.

In other aspects of this embodiment, a modified Clostridial toxin comprises a BoNT/D di-chain loop region having, e.g., at least 50% amino acid identity with SEQ ID NO: 14, at least 60% amino acid identity with the SEQ ID NO: 14, at least 70% amino acid identity with SEQ ID NO: 14, at least 80% amino acid identity with SEQ ID NO: 14, or at least 90% amino acid identity with SEQ ID NO: 14. In still other aspects of this embodiment, a modified Clostridial toxin comprises a BoNT/D di-chain loop region having, e.g., at most 50% amino acid identity with SEQ ID NO: 14, at most 60% amino acid identity with the SEQ ID NO: 14, at most 70% amino acid identity with SEQ ID NO: 14, at most 80% amino acid identity with SEQ ID NO: 14, or at most 90% amino acid identity with SEQ ID NO: 14.

In other aspects of this embodiment, a modified Clostridial toxin comprises a BoNT/D di-chain loop region having, e.g., at most one, two, three, four, five, six, or seven non-contiguous amino acid substitutions relative to SEQ ID NO: 14. In still other aspects of this embodiment, a modified Clostridial toxin comprises a BoNT/D di-chain loop region having, e.g., at least one, two, three, four, five, six, or seven non-contiguous amino acid substitutions relative to SEQ ID NO: 14. In yet other aspects of this embodiment, a modified Clostridial toxin comprises a BoNT/D di-chain loop region having, e.g., at most one, two, three, four, five, six, or seven non-contiguous amino acid additions relative to SEQ ID NO: 14. In yet other aspects of this embodiment, a modified Clostridial toxin comprises a BoNT/D di-chain loop region having, e.g., at least one, two, three, four, five, six, or seven non-contiguous amino acid additions relative to SEQ ID NO: 14. In still other aspects of this embodiment, a modified Clostridial toxin comprises a BoNT/D di-chain loop region having, e.g., at most one, two, three, four, five, six, or seven non-contiguous amino acid deletions relative to SEQ ID NO: 14. In still other aspects of this embodiment, a modified Clostridial toxin comprises a BoNT/D di-chain loop region having, e.g., at least one, two, three, four, five, six, or seven non-contiguous amino acid deletions relative to SEQ ID NO: 14.

In other aspects of this embodiment, a modified Clostridial toxin comprises a BoNT/D di-chain loop region having, e.g., at most one, two, three, four, five, six, or seven contiguous amino acid substitutions relative to SEQ ID NO: 14. In still other aspects of this embodiment, a modified Clostridial toxin comprises a BoNT/D di-chain loop region having, e.g., at least one, two, three, four, five, six, or seven contiguous amino acid substitutions relative to SEQ ID NO: 14. In yet other aspects of this embodiment, a modified Clostridial toxin comprises a BoNT/D di-chain loop region having, e.g., at most one, two, three, four, five, six, or seven contiguous amino acid additions relative to SEQ ID NO: 14. In yet other aspects of this embodiment, a modified Clostridial toxin comprises a BoNT/D di-chain loop region having, e.g., at least one, two, three, four, five, six, or seven contiguous amino acid additions relative to SEQ ID NO: 14. In still other aspects of this embodiment, a modified Clostridial toxin comprises a BoNT/D di-chain loop region having, e.g., at most one, two, three, four, five, six, or seven contiguous amino acid deletions relative to SEQ ID NO: 14. In still other aspects of this embodiment, a modified Clostridial toxin comprises a BoNT/D di-chain loop region having, e.g., at least one, two, three, four, five, six, or seven contiguous amino acid deletions relative to SEQ ID NO: 14.

In another embodiment, a modified Clostridial toxin comprises a BoNT/E di-chain loop region. In an aspect of this embodiment, a modified Clostridial toxin comprises a BoNT/E di-chain loop region including a BoNT/E di-chain loop protease cleavage site. In another aspect of this embodiment, a modified Clostridial toxin comprises a BoNT/E di-chain loop region including a BoNT/E di-chain loop protease cleavage site comprising the K419-G420 scissile bond. In another aspect of this embodiment, a modified Clostridial toxin comprises a BoNT/E di-chain loop region including a BoNT/E di-chain loop protease cleavage site comprising the R422-K423 scissile bond. In another aspect of this embodiment, a modified Clostridial toxin comprises a BoNT/E di-chain loop region including a BoNT/E di-chain loop protease cleavage site comprising the K423-5424 scissile bond. In yet another aspect of this embodiment, a modified Clostridial toxin comprises the BoNT/E di-chain loop region of SEQ ID NO: 15.

In another aspect of this embodiment, a modified Clostridial toxin comprises a naturally occurring BoNT/E di-chain loop region variant. In another aspect of this embodiment, a modified Clostridial toxin comprises a naturally occurring BoNT/E di-chain loop region variant, such as, e.g., a BoNT/E di-chain loop region isoform, or a BoNT/E di-chain loop region subtype. In another aspect of this embodiment, a modified Clostridial toxin comprises a naturally occurring BoNT/E di-chain loop region variant of SEQ ID NO: 15, such as, e.g., a BoNT/E di-chain loop region isoform of SEQ ID NO: 15; or a BoNT/E di-chain loop region subtype of SEQ ID NO: 15. In still another aspect of this embodiment, a modified Clostridial toxin comprises a non-naturally occurring BoNT/E di-chain loop region variant, such as, e.g., a conservative BoNT/E di-chain loop region variant, a non-conservative BoNT/E di-chain loop region variant or a BoNT/E di-chain loop region peptidomimetic, or any combination thereof. In still another aspect of this embodiment, a modified Clostridial toxin comprises a non-naturally occurring BoNT/E di-chain loop region variant of SEQ ID NO: 15, such as, e.g., a conservative BoNT/E di-chain loop region variant of SEQ ID NO: 15, a non-conservative BoNT/E di-chain loop region variant of SEQ ID NO: 15 or a BoNT/E di-chain loop region peptidomimetic of SEQ ID NO: 15, or any combination thereof.

In other aspects of this embodiment, a modified Clostridial toxin comprises a BoNT/E di-chain loop region having, e.g., at least 50% amino acid identity with SEQ ID NO: 15, at least 60% amino acid identity with the SEQ ID NO: 15, at least 70% amino acid identity with SEQ ID NO: 15, at least 80% amino acid identity with SEQ ID NO: 15, or at least 90% amino acid identity with SEQ ID NO: 15. In still other aspects of this embodiment, a modified Clostridial toxin comprises a BoNT/E di-chain loop region having, e.g., at most 50% amino acid identity with SEQ ID NO: 15, at most 60% amino acid identity with the SEQ ID NO: 15, at most 70% amino acid identity with SEQ ID NO: 15, at most 80% amino acid identity with SEQ ID NO: 15, or at most 90% amino acid identity with SEQ ID NO: 15.

In other aspects of this embodiment, a modified Clostridial toxin comprises a BoNT/E di-chain loop region having, e.g., at most one, two, three, four, five, six, or seven non-contiguous amino acid substitutions relative to SEQ ID NO: 15. In still other aspects of this embodiment, a modified Clostridial toxin comprises a BoNT/E di-chain loop region having, e.g., at least one, two, three, four, five, six, or seven non-contiguous amino acid substitutions relative to SEQ ID NO: 15. In yet other aspects of this embodiment, a modified Clostridial toxin comprises a BoNT/E di-chain loop region having, e.g., at most one, two, three, four, five, six, or seven non-contiguous amino acid additions relative to SEQ ID NO: 15. In yet other aspects of this embodiment, a modified Clostridial toxin comprises a BoNT/E di-chain loop region having, e.g., at least one, two, three, four, five, six, or seven non-contiguous amino acid additions relative to SEQ ID NO: 15. In still other aspects of this embodiment, a modified Clostridial toxin comprises a BoNT/E di-chain loop region having, e.g., at most one, two, three, four, five, six, or seven non-contiguous amino acid deletions relative to SEQ ID NO: 15. In still other aspects of this embodiment, a modified Clostridial toxin comprises a BoNT/E di-chain loop region having, e.g., at least one, two, three, four, five, six, or seven non-contiguous amino acid deletions relative to SEQ ID NO: 15.

In other aspects of this embodiment, a modified Clostridial toxin comprises a BoNT/E di-chain loop region having, e.g., at most one, two, three, four, five, six, or seven contiguous amino acid substitutions relative to SEQ ID NO: 15. In still other aspects of this embodiment, a modified Clostridial toxin comprises a BoNT/E di-chain loop region having, e.g., at least one, two, three, four, five, six, or seven contiguous amino acid substitutions relative to SEQ ID NO: 15. In yet other aspects of this embodiment, a modified Clostridial toxin comprises a BoNT/E di-chain loop region having, e.g., at most one, two, three, four, five, six, or seven contiguous amino acid additions relative to SEQ ID NO: 15. In yet other aspects of this embodiment, a modified Clostridial toxin comprises a BoNT/E di-chain loop region having, e.g., at least one, two, three, four, five, six, or seven contiguous amino acid additions relative to SEQ ID NO: 15. In still other aspects of this embodiment, a modified Clostridial toxin comprises a BoNT/E di-chain loop region having, e.g., at most one, two, three, four, five, six, or seven contiguous amino acid deletions relative to SEQ ID NO: 15. In still other aspects of this embodiment, a modified Clostridial toxin comprises a BoNT/E di-chain loop region having, e.g., at least one, two, three, four, five, six, or seven contiguous amino acid deletions relative to SEQ ID NO: 15.

In another embodiment, a modified Clostridial toxin comprises a BoNT/F di-chain loop region. In an aspect of this embodiment, a modified Clostridial toxin comprises a BoNT/F di-chain loop region including a BoNT/F di-chain loop protease cleavage site. In another aspect of this embodiment, a modified Clostridial toxin comprises a BoNT/F di-chain loop region including a BoNT/F di-chain loop protease cleavage site comprising the K436-G437 scissile bond. In another aspect of this embodiment, a modified Clostridial toxin comprises a BoNT/F di-chain loop region including a BoNT/F di-chain loop protease cleavage site comprising the K439-A440 scissile bond. In yet another aspect of this embodiment, a modified Clostridial toxin comprises the BoNT/F di-chain loop region of SEQ ID NO: 16.

In another aspect of this embodiment, a modified Clostridial toxin comprises a naturally occurring BoNT/F di-chain loop region variant. In another aspect of this embodiment, a modified Clostridial toxin comprises a naturally occurring BoNT/F di-chain loop region variant, such as, e.g., a BoNT/F di-chain loop region isoform, or a BoNT/F di-chain loop region subtype. In another aspect of this embodiment, a modified Clostridial toxin comprises a naturally occurring BoNT/F di-chain loop region variant of SEQ ID NO: 16, such as, e.g., a BoNT/F di-chain loop region isoform of SEQ ID NO: 16; or a BoNT/F di-chain loop region subtype of SEQ ID NO: 16. In still another aspect of this embodiment, a modified Clostridial toxin comprises a non-naturally occurring BoNT/F di-chain loop region variant, such as, e.g., a conservative BoNT/F di-chain loop region variant, a non-conservative BoNT/F di-chain loop region variant or a BoNT/F di-chain loop region peptidomimetic, or any combination thereof. In still another aspect of this embodiment, a modified Clostridial toxin comprises a non-naturally occurring BoNT/F di-chain loop region variant of SEQ ID NO: 16, such as, e.g., a conservative BoNT/F di-chain loop region variant of SEQ ID NO: 16, a non-conservative BoNT/F di-chain loop region variant of SEQ ID NO: 16 or a BoNT/F di-chain loop region peptidomimetic of SEQ ID NO: 16, or any combination thereof.

In other aspects of this embodiment, a modified Clostridial toxin comprises a BoNT/F di-chain loop region having, e.g., at least 50% amino acid identity with SEQ ID NO: 16, at least 60% amino acid identity with the SEQ ID NO: 16, at least 70% amino acid identity with SEQ ID NO: 16, at least 80% amino acid identity with SEQ ID NO: 16, or at least 90% amino acid identity with SEQ ID NO: 16. In still other aspects of this embodiment, a modified Clostridial toxin comprises a BoNT/F di-chain loop region having, e.g., at most 50% amino acid identity with SEQ ID NO: 16, at most 60% amino acid identity with the SEQ ID NO: 16, at most 70% amino acid identity with SEQ ID NO: 16, at most 80% amino acid identity with SEQ ID NO: 16, or at most 90% amino acid identity with SEQ ID NO: 16.

In other aspects of this embodiment, a modified Clostridial toxin comprises a BoNT/F di-chain loop region having, e.g., at most one, two, three, four, five, six, seven, eight, nine or ten non-contiguous amino acid substitutions relative to SEQ ID NO: 16. In still other aspects of this embodiment, a modified Clostridial toxin comprises a BoNT/F di-chain loop region having, e.g., at least one, two, three, four, five, six, seven, eight, nine or ten non-contiguous amino acid substitutions relative to SEQ ID NO: 16. In yet other aspects of this embodiment, a modified Clostridial toxin comprises a BoNT/F di-chain loop region having, e.g., at most one, two, three, four, five, six, seven, eight, nine or ten non-contiguous amino acid additions relative to SEQ ID NO: 16. In yet other aspects of this embodiment, a modified Clostridial toxin comprises a BoNT/F di-chain loop region having, e.g., at least one, two, three, four, five, six, seven, eight, nine or ten non-contiguous amino acid additions relative to SEQ ID NO: 16. In still other aspects of this embodiment, a modified Clostridial toxin comprises a BoNT/F di-chain loop region having, e.g., at most one, two, three, four, five, six, seven, eight, nine or ten non-contiguous amino acid deletions relative to SEQ ID NO: 16. In still other aspects of this embodiment, a modified Clostridial toxin comprises a BoNT/F di-chain loop region having, e.g., at least one, two, three, four, five, six, seven, eight, nine or ten non-contiguous amino acid deletions relative to SEQ ID NO: 16.

In other aspects of this embodiment, a modified Clostridial toxin comprises a BoNT/F di-chain loop region having, e.g., at most one, two, three, four, five, six, seven, eight, nine or ten contiguous amino acid substitutions relative to SEQ ID NO: 16. In still other aspects of this embodiment, a modified Clostridial toxin comprises a BoNT/F di-chain loop region having, e.g., at least one, two, three, four, five, six, seven, eight, nine or ten contiguous amino acid substitutions relative to SEQ ID NO: 16. In yet other aspects of this embodiment, a modified Clostridial toxin comprises a BoNT/F di-chain loop region having, e.g., at most one, two, three, four, five, six, seven, eight, nine or ten contiguous amino acid additions relative to SEQ ID NO: 16. In yet other aspects of this embodiment, a modified Clostridial toxin comprises a BoNT/F di-chain loop region having, e.g., at least one, two, three, four, five, six, seven, eight, nine or ten contiguous amino acid additions relative to SEQ ID NO: 16. In still other aspects of this embodiment, a modified Clostridial toxin comprises a BoNT/F di-chain loop region having, e.g., at most one, two, three, four, five, six, seven, eight, nine or ten contiguous amino acid deletions relative to SEQ ID NO: 16. In still other aspects of this embodiment, a modified Clostridial toxin comprises a BoNT/F di-chain loop region having, e.g., at least one, two, three, four, five, six, seven, eight, nine or ten contiguous amino acid deletions relative to SEQ ID NO: 16.

In another embodiment, a modified Clostridial toxin comprises a BoNT/G di-chain loop region. In an aspect of this embodiment, a modified Clostridial toxin comprises a BoNT/G di-chain loop region including a BoNT/G di-chain loop protease cleavage site. In another aspect of this embodiment, a modified Clostridial toxin comprises a BoNT/G di-chain loop region including a BoNT/G di-chain loop protease cleavage site comprising the T444-G445 scissile bond. In another aspect of this embodiment, a modified Clostridial toxin comprises a BoNT/G di-chain loop region including a BoNT/G di-chain loop protease cleavage site comprising the K446-S447 scissile bond. In another aspect of this embodiment, a modified Clostridial toxin comprises a BoNT/G di-chain loop region including a BoNT/G di-chain loop protease cleavage site comprising the E448-Q449 scissile bond. In yet another aspect of this embodiment, a modified Clostridial toxin comprises the BoNT/G di-chain loop region of SEQ ID NO: 17.

In another aspect of this embodiment, a modified Clostridial toxin comprises a naturally occurring BoNT/G di-chain loop region variant. In another aspect of this embodiment, a modified Clostridial toxin comprises a naturally occurring BoNT/G di-chain loop region variant, such as, e.g., a BoNT/G di-chain loop region isoform, or a BoNT/G di-chain loop region subtype. In another aspect of this embodiment, a modified Clostridial toxin comprises a naturally occurring BoNT/G di-chain loop region variant of SEQ ID NO: 17, such as, e.g., a BoNT/G di-chain loop region isoform of SEQ ID NO: 17; or a BoNT/G di-chain loop region subtype of SEQ ID NO: 17. In still another aspect of this embodiment, a modified Clostridial toxin comprises a non-naturally occurring BoNT/G di-chain loop region variant, such as, e.g., a conservative BoNT/G di-chain loop region variant, a non-conservative BoNT/G di-chain loop region variant or a BoNT/G di-chain loop region peptidomimetic, or any combination thereof. In still another aspect of this embodiment, a modified Clostridial toxin comprises a non-naturally occurring BoNT/G di-chain loop region variant of SEQ ID NO: 17, such as, e.g., a conservative BoNT/G di-chain loop region variant of SEQ ID NO: 17, a non-conservative BoNT/G di-chain loop region variant of SEQ ID NO: 17 or a BoNT/G di-chain loop region peptidomimetic of SEQ ID NO: 17, or any combination thereof.

In other aspects of this embodiment, a modified Clostridial toxin comprises a BoNT/G di-chain loop region having, e.g., at least 50% amino acid identity with SEQ ID NO: 17, at least 60% amino acid identity with the SEQ ID NO: 17, at least 70% amino acid identity with SEQ ID NO: 17, at least 80% amino acid identity with SEQ ID NO: 17, or at least 90% amino acid identity with SEQ ID NO: 17. In still other aspects of this embodiment, a modified Clostridial toxin comprises a BoNT/G di-chain loop region having, e.g., at most 50% amino acid identity with SEQ ID NO: 17, at most 60% amino acid identity with the SEQ ID NO: 17, at most 70% amino acid identity with SEQ ID NO: 17, at most 80% amino acid identity with SEQ ID NO: 17, or at most 90% amino acid identity with SEQ ID NO: 17.

In other aspects of this embodiment, a modified Clostridial toxin comprises a BoNT/G di-chain loop region having, e.g., at most one, two, three, four, five, six, or seven non-contiguous amino acid substitutions relative to SEQ ID NO: 17. In still other aspects of this embodiment, a modified Clostridial toxin comprises a BoNT/G di-chain loop region having, e.g., at least one, two, three, four, five, six, or seven non-contiguous amino acid substitutions relative to SEQ ID NO: 17. In yet other aspects of this embodiment, a modified Clostridial toxin comprises a BoNT/G di-chain loop region having, e.g., at most one, two, three, four, five, six, or seven non-contiguous amino acid additions relative to SEQ ID NO: 17. In yet other aspects of this embodiment, a modified Clostridial toxin comprises a BoNT/G di-chain loop region having, e.g., at least one, two, three, four, five, six, or seven non-contiguous amino acid additions relative to SEQ ID NO: 17. In still other aspects of this embodiment, a modified Clostridial toxin comprises a BoNT/G di-chain loop region having, e.g., at most one, two, three, four, five, six, or seven non-contiguous amino acid deletions relative to SEQ ID NO: 17. In still other aspects of this embodiment, a modified Clostridial toxin comprises a BoNT/G di-chain loop region having, e.g., at least one, two, three, four, five, six, or seven non-contiguous amino acid deletions relative to SEQ ID NO: 17.

In other aspects of this embodiment, a modified Clostridial toxin comprises a BoNT/G di-chain loop region having, e.g., at most one, two, three, four, five, six, or seven contiguous amino acid substitutions relative to SEQ ID NO: 17. In still other aspects of this embodiment, a modified Clostridial toxin comprises a BoNT/G di-chain loop region having, e.g., at least one, two, three, four, five, six, or seven contiguous amino acid substitutions relative to SEQ ID NO: 17. In yet other aspects of this embodiment, a modified Clostridial toxin comprises a BoNT/G di-chain loop region having, e.g., at most one, two, three, four, five, six, or seven contiguous amino acid additions relative to SEQ ID NO: 17. In yet other aspects of this embodiment, a modified Clostridial toxin comprises a BoNT/G di-chain loop region having, e.g., at least one, two, three, four, five, six, or seven contiguous amino acid additions relative to SEQ ID NO: 17. In still other aspects of this embodiment, a modified Clostridial toxin comprises a BoNT/G di-chain loop region having, e.g., at most one, two, three, four, five, six, or seven contiguous amino acid deletions relative to SEQ ID NO: 17. In still other aspects of this embodiment, a modified Clostridial toxin comprises a BoNT/G di-chain loop region having, e.g., at least one, two, three, four, five, six, or seven contiguous amino acid deletions relative to SEQ ID NO: 17.

In another embodiment, a modified Clostridial toxin comprises a TeNT di-chain loop region. In an aspect of this embodiment, a modified Clostridial toxin comprises a TeNT di-chain loop region including a TeNT di-chain loop protease cleavage site. In another aspect of this embodiment, a modified Clostridial toxin comprises a TeNT di-chain loop region including a TeNT di-chain loop protease cleavage site comprising the A457-S458 scissile bond. In yet another aspect of this embodiment, a modified Clostridial toxin comprises the TeNT di-chain loop region of SEQ ID NO: 18.

In another aspect of this embodiment, a modified Clostridial toxin comprises a naturally occurring TeNT di-chain loop region variant. In another aspect of this embodiment, a modified Clostridial toxin comprises a naturally occurring TeNT di-chain loop region variant, such as, e.g., a TeNT di-chain loop region isoform, or a TeNT di-chain loop region subtype. In another aspect of this embodiment, a modified Clostridial toxin comprises a naturally occurring TeNT di-chain loop region variant of SEQ ID NO: 18, such as, e.g., a TeNT di-chain loop region isoform of SEQ ID NO: 18; or a TeNT di-chain loop region subtype of SEQ ID NO: 18. In still another aspect of this embodiment, a modified Clostridial toxin comprises a non-naturally occurring TeNT di-chain loop region variant, such as, e.g., a conservative TeNT di-chain loop region variant, a non-conservative TeNT di-chain loop region variant or a TeNT di-chain loop region peptidomimetic, or any combination thereof. In still another aspect of this embodiment, a modified Clostridial toxin comprises a non-naturally occurring TeNT di-chain loop region variant of SEQ ID NO: 18, such as, e.g., a conservative TeNT di-chain loop region variant of SEQ ID NO: 18, a non-conservative TeNT di-chain loop region variant of SEQ ID NO: 18 or a TeNT di-chain loop region peptidomimetic of SEQ ID NO: 18, or any combination thereof.

In other aspects of this embodiment, a modified Clostridial toxin comprises a TeNT di-chain loop region having, e.g., at least 50% amino acid identity with SEQ ID NO: 18, at least 60% amino acid identity with the SEQ ID NO: 18, at least 70% amino acid identity with SEQ ID NO: 18, at least 80% amino acid identity with SEQ ID NO: 18, or at least 90% amino acid identity with SEQ ID NO: 18. In still other aspects of this embodiment, a modified Clostridial toxin comprises a TeNT di-chain loop region having, e.g., at most 50% amino acid identity with SEQ ID NO: 18, at most 60% amino acid identity with the SEQ ID NO: 18, at most 70% amino acid identity with SEQ ID NO: 18, at most 80% amino acid identity with SEQ ID NO: 18, or at most 90% amino acid identity with SEQ ID NO: 18.

In other aspects of this embodiment, a modified Clostridial toxin comprises a TeNT di-chain loop region having, e.g., at most one, two, three, four, five, six, seven, eight, nine or ten non-contiguous amino acid substitutions relative to SEQ ID NO: 18. In still other aspects of this embodiment, a modified Clostridial toxin comprises a TeNT di-chain loop region having, e.g., at least one, two, three, four, five, six, seven, eight, nine or ten non-contiguous amino acid substitutions relative to SEQ ID NO: 18. In yet other aspects of this embodiment, a modified Clostridial toxin comprises a TeNT di-chain loop region having, e.g., at most one, two, three, four, five, six, seven, eight, nine or ten non-contiguous amino acid additions relative to SEQ ID NO: 18. In yet other aspects of this embodiment, a modified Clostridial toxin comprises a TeNT di-chain loop region having, e.g., at least one, two, three, four, five, six, seven, eight, nine or ten non-contiguous amino acid additions relative to SEQ ID NO: 18. In still other aspects of this embodiment, a modified Clostridial toxin comprises a TeNT di-chain loop region having, e.g., at most one, two, three, four, five, six, seven, eight, nine or ten non-contiguous amino acid deletions relative to SEQ ID NO: 18. In still other aspects of this embodiment, a modified Clostridial toxin comprises a TeNT di-chain loop region having, e.g., at least one, two, three, four, five, six, seven, eight, nine or ten non-contiguous amino acid deletions relative to SEQ ID NO: 18.

In other aspects of this embodiment, a modified Clostridial toxin comprises a TeNT di-chain loop region having, e.g., at most one, two, three, four, five, six, seven, eight, nine or ten contiguous amino acid substitutions relative to SEQ ID NO: 18. In still other aspects of this embodiment, a modified Clostridial toxin comprises a TeNT di-chain loop region having, e.g., at least one, two, three, four, five, six, seven, eight, nine or ten contiguous amino acid substitutions relative to SEQ ID NO: 18. In yet other aspects of this embodiment, a modified Clostridial toxin comprises a TeNT di-chain loop region having, e.g., at most one, two, three, four, five, six, seven, eight, nine or ten contiguous amino acid additions relative to SEQ ID NO: 18. In yet other aspects of this embodiment, a modified Clostridial toxin comprises a TeNT di-chain loop region having, e.g., at least one, two, three, four, five, six, seven, eight, nine or ten contiguous amino acid additions relative to SEQ ID NO: 18. In still other aspects of this embodiment, a modified Clostridial toxin comprises a TeNT di-chain loop region having, e.g., at most one, two, three, four, five, six, seven, eight, nine or ten contiguous amino acid deletions relative to SEQ ID NO: 18. In still other aspects of this embodiment, a modified Clostridial toxin comprises a TeNT di-chain loop region having, e.g., at least one, two, three, four, five, six, seven, eight, nine or ten contiguous amino acid deletions relative to SEQ ID NO: 18.

In another embodiment, a modified Clostridial toxin comprises a BaNT di-chain loop region. In an aspect of this embodiment, a modified Clostridial toxin comprises a BaNT di-chain loop region including a BaNT di-chain loop protease cleavage site. In another aspect of this embodiment, a modified Clostridial toxin comprises a BaNT di-chain loop region including a BaNT di-chain loop protease cleavage site comprising the K431-N432 scissile bond. In yet another aspect of this embodiment, a modified Clostridial toxin comprises the BaNT di-chain loop region of SEQ ID NO: 19.

In another aspect of this embodiment, a modified Clostridial toxin comprises a naturally occurring BaNT di-chain loop region variant. In another aspect of this embodiment, a modified Clostridial toxin comprises a naturally occurring BaNT di-chain loop region variant, such as, e.g., a BaNT di-chain loop region isoform, or a BaNT di-chain loop region subtype. In another aspect of this embodiment, a modified Clostridial toxin comprises a naturally occurring BaNT di-chain loop region variant of SEQ ID NO: 19, such as, e.g., a BaNT di-chain loop region isoform of SEQ ID NO: 19; or a BaNT di-chain loop region subtype of SEQ ID NO: 19. In still another aspect of this embodiment, a modified Clostridial toxin comprises a non-naturally occurring BaNT di-chain loop region variant, such as, e.g., a conservative BaNT di-chain loop region variant, a non-conservative BaNT di-chain loop region variant or a BaNT di-chain loop region peptidomimetic, or any combination thereof. In still another aspect of this embodiment, a modified Clostridial toxin comprises a non-naturally occurring BaNT di-chain loop region variant of SEQ ID NO: 19, such as, e.g., a conservative BaNT di-chain loop region variant of SEQ ID NO: 19, a non-conservative BaNT di-chain loop region variant of SEQ ID NO: 19 or a BaNT di-chain loop region peptidomimetic of SEQ ID NO: 19, or any combination thereof.

In other aspects of this embodiment, a modified Clostridial toxin comprises a BaNT di-chain loop region having, e.g., at least 50% amino acid identity with SEQ ID NO: 19, at least 60% amino acid identity with the SEQ ID NO: 19, at least 70% amino acid identity with SEQ ID NO: 19, at least 80% amino acid identity with SEQ ID NO: 19, or at least 90% amino acid identity with SEQ ID NO: 19. In still other aspects of this embodiment, a modified Clostridial toxin comprises a BaNT di-chain loop region having, e.g., at most 50% amino acid identity with SEQ ID NO: 19, at most 60% amino acid identity with the SEQ ID NO: 19, at most 70% amino acid identity with SEQ ID NO: 19, at most 80% amino acid identity with SEQ ID NO: 19, or at most 90% amino acid identity with SEQ ID NO: 19.

In other aspects of this embodiment, a modified Clostridial toxin comprises a BaNT di-chain loop region having, e.g., at most one, two, three, four, five, six, or seven non-contiguous amino acid substitutions relative to SEQ ID NO: 19. In still other aspects of this embodiment, a modified Clostridial toxin comprises a BaNT di-chain loop region having, e.g., at least one, two, three, four, five, six, or seven non-contiguous amino acid substitutions relative to SEQ ID NO: 19. In yet other aspects of this embodiment, a modified Clostridial toxin comprises a BaNT di-chain loop region having, e.g., at most one, two, three, four, five, six, or seven non-contiguous amino acid additions relative to SEQ ID NO: 19. In yet other aspects of this embodiment, a modified Clostridial toxin comprises a BaNT di-chain loop region having, e.g., at least one, two, three, four, five, six, or seven non-contiguous amino acid additions relative to SEQ ID NO: 19. In still other aspects of this embodiment, a modified Clostridial toxin comprises a BaNT di-chain loop region having, e.g., at most one, two, three, four, five, six, or seven non-contiguous amino acid deletions relative to SEQ ID NO: 19. In still other aspects of this embodiment, a modified Clostridial toxin comprises a BaNT di-chain loop region having, e.g., at least one, two, three, four, five, six, or seven non-contiguous amino acid deletions relative to SEQ ID NO: 19.

In other aspects of this embodiment, a modified Clostridial toxin comprises a BaNT di-chain loop region having, e.g., at most one, two, three, four, five, six, or seven contiguous amino acid substitutions relative to SEQ ID NO: 19. In still other aspects of this embodiment, a modified Clostridial toxin comprises a BaNT di-chain loop region having, e.g., at least one, two, three, four, five, six, or seven contiguous amino acid substitutions relative to SEQ ID NO: 19. In yet other aspects of this embodiment, a modified Clostridial toxin comprises a BaNT di-chain loop region having, e.g., at most one, two, three, four, five, six, or seven contiguous amino acid additions relative to SEQ ID NO: 19. In yet other aspects of this embodiment, a modified Clostridial toxin comprises a BaNT di-chain loop region having, e.g., at least one, two, three, four, five, six, or seven contiguous amino acid additions relative to SEQ ID NO: 19. In still other aspects of this embodiment, a modified Clostridial toxin comprises a BaNT di-chain loop region having, e.g., at most one, two, three, four, five, six, or seven contiguous amino acid deletions relative to SEQ ID NO: 19. In still other aspects of this embodiment, a modified Clostridial toxin comprises a BaNT di-chain loop region having, e.g., at least one, two, three, four, five, six, or seven contiguous amino acid deletions relative to SEQ ID NO: 19.

In another embodiment, a modified Clostridial toxin comprises a BuNT di-chain loop region. In an aspect of this embodiment, a modified Clostridial toxin comprises a BuNT di-chain loop region including a BuNT di-chain loop protease cleavage site. In another aspect of this embodiment, a modified Clostridial toxin comprises a BuNT di-chain loop region including a BuNT di-chain loop protease cleavage site comprising the K431-N432 scissile bond. In yet another aspect of this embodiment, a modified Clostridial toxin comprises the BuNT di-chain loop region of SEQ ID NO: 20.

In another aspect of this embodiment, a modified Clostridial toxin comprises a naturally occurring BuNT di-chain loop region variant. In another aspect of this embodiment, a modified Clostridial toxin comprises a naturally occurring BuNT di-chain loop region variant, such as, e.g., a BuNT di-chain loop region isoform, or a BuNT di-chain loop region subtype. In another aspect of this embodiment, a modified Clostridial toxin comprises a naturally occurring BuNT di-chain loop region variant of SEQ ID NO: 20, such as, e.g., a BuNT di-chain loop region isoform of SEQ ID NO: 20; or a BuNT di-chain loop region subtype of SEQ ID NO: 20. In still another aspect of this embodiment, a modified Clostridial toxin comprises a non-naturally occurring BuNT di-chain loop region variant, such as, e.g., a conservative BuNT di-chain loop region variant, a non-conservative BuNT di-chain loop region variant or a BuNT di-chain loop region peptidomimetic, or any combination thereof. In still another aspect of this embodiment, a modified Clostridial toxin comprises a non-naturally occurring BuNT di-chain loop region variant of SEQ ID NO: 20, such as, e.g., a conservative BuNT di-chain loop region variant of SEQ ID NO: 20, a non-conservative BuNT di-chain loop region variant of SEQ ID NO: 20 or a BuNT di-chain loop region peptidomimetic of SEQ ID NO: 20, or any combination thereof.

In other aspects of this embodiment, a modified Clostridial toxin comprises a BuNT di-chain loop region having, e.g., at least 50% amino acid identity with SEQ ID NO: 20, at least 60% amino acid identity with the SEQ ID NO: 20, at least 70% amino acid identity with SEQ ID NO: 20, at least 80% amino acid identity with SEQ ID NO: 20, or at least 90% amino acid identity with SEQ ID NO: 20. In still other aspects of this embodiment, a modified Clostridial toxin comprises a BuNT di-chain loop region having, e.g., at most 50% amino acid identity with SEQ ID NO: 20, at most 60% amino acid identity with the SEQ ID NO: 20, at most 70% amino acid identity with SEQ ID NO: 20, at most 80% amino acid identity with SEQ ID NO: 20, or at most 90% amino acid identity with SEQ ID NO: 20.

In other aspects of this embodiment, a modified Clostridial toxin comprises a BuNT di-chain loop region having, e.g., at most one, two, three, four, five, six, or seven non-contiguous amino acid substitutions relative to SEQ ID NO: 20. In still other aspects of this embodiment, a modified Clostridial toxin comprises a BuNT di-chain loop region having, e.g., at least one, two, three, four, five, six, or seven non-contiguous amino acid substitutions relative to SEQ ID NO: 20. In yet other aspects of this embodiment, a modified Clostridial toxin comprises a BuNT di-chain loop region having, e.g., at most one, two, three, four, five, six, or seven non-contiguous amino acid additions relative to SEQ ID NO: 20. In yet other aspects of this embodiment, a modified Clostridial toxin comprises a BuNT di-chain loop region having, e.g., at least one, two, three, four, five, six, or seven non-contiguous amino acid additions relative to SEQ ID NO: 20. In still other aspects of this embodiment, a modified Clostridial toxin comprises a BuNT di-chain loop region having, e.g., at most one, two, three, four, five, six, or seven non-contiguous amino acid deletions relative to SEQ ID NO: 20. In still other aspects of this embodiment, a modified Clostridial toxin comprises a BuNT di-chain loop region having, e.g., at least one, two, three, four, five, six, or seven non-contiguous amino acid deletions relative to SEQ ID NO: 20.

In other aspects of this embodiment, a modified Clostridial toxin comprises a BuNT di-chain loop region having, e.g., at most one, two, three, four, five, six, or seven contiguous amino acid substitutions relative to SEQ ID NO: 20. In still other aspects of this embodiment, a modified Clostridial toxin comprises a BuNT di-chain loop region having, e.g., at least one, two, three, four, five, six, or seven contiguous amino acid substitutions relative to SEQ ID NO: 20. In yet other aspects of this embodiment, a modified Clostridial toxin comprises a BuNT di-chain loop region having, e.g., at most one, two, three, four, five, six, or seven contiguous amino acid additions relative to SEQ ID NO: 20. In yet other aspects of this embodiment, a modified Clostridial toxin comprises a BuNT di-chain loop region having, e.g., at least one, two, three, four, five, six, or seven contiguous amino acid additions relative to SEQ ID NO: 20. In still other aspects of this embodiment, a modified Clostridial toxin comprises a BuNT di-chain loop region having, e.g., at most one, two, three, four, five, six, or seven contiguous amino acid deletions relative to SEQ ID NO: 20. In still other aspects of this embodiment, a modified Clostridial toxin comprises a BuNT di-chain loop region having, e.g., at least one, two, three, four, five, six, or seven contiguous amino acid deletions relative to SEQ ID NO: 20.

The di-chain loop region of the Clostridial toxin to be modified can be modified to include an exogenous Clostridial toxin di-chain loop region in addition to the naturally-occurring di-chain loop region (Table 3). In this type of modification, both di-chain loop regions are operably-linked in-frame to the modified Clostridial toxin as a fusion protein and both sites can be cleaved by their respective proteases. In such a modification, the cysteine residues from the exogenous di-chain loop region should not be included because the additional cysteine residues could interfere with the proper formation of the disulfide bridge necessary to for the loop structure. As a non-limiting example, a modified BoNT/E can comprise a di-chain loop containing both the naturally-occurring di-chain loop region and a BoNT/A di-chain loop region (e.g., SEQ ID NO: 11 minus the cysteine residues at position 1 and position 25) that can be cleaved by a BoNT/A di-chain loop protease found in C. botulinum serotype A.

TABLE 3
Examples of Modified Clostridial Toxins

| | Di-Chain | | |
|---|---|---|---|
| | Loop | | |
| Enzymatic Domain | Region¹ | Translocation Domain | Binding Domain |
| BoNT/B, BoNT/C1, | BoNT/A | BoNT/B, BoNT/C1, BoNT/D, | BoNT/B, BoNT/C1, BoNT/D, |
| BoNT/D, BoNT/E, BoNT/F, | | BoNT/E, BoNT/F, BoNT/G, | BoNT/E, BoNT/F, BoNT/G, |
| BoNT/G, TeNT, BaNT, or | | TeNT, BaNT, or BuNT | TeNT, BaNT, BuNT, or |
| BuNT | | | targeting moiety² |
| BoNT/A, BoNT/C1, | BoNT/B | BoNT/A, BoNT/C1, BoNT/D, | BoNT/A, BoNT/C1, BoNT/D, |
| BoNT/D, BoNT/E, BoNT/F, | | BoNT/E, BoNT/F, BoNT/G, | BoNT/E, BoNT/F, BoNT/G, |
| BoNT/G, TeNT, BaNT, or | | TeNT, BaNT, or BuNT | TeNT, BaNT, BuNT, or re- |
| BuNT | | | targeting moeity |
| BoNT/A, BoNT/B, BoNT/D, | BoNT/C1 | BoNT/A, BoNT/B, BoNT/D, | BoNT/A, BoNT/B, BoNT/D, |
| BoNT/E, BoNT/F, BoNT/G, | | BoNT/E, BoNT/F, BoNT/G, | BoNT/E, BoNT/F, BoNT/G, |
| TeNT, BaNT, or BuNT | | TeNT, BaNT, or BuNT | TeNT, BaNT, BuNT, or re- |
| | | | targeting moeity |
| BoNT/A, BoNT/B, | BoNT/D | BoNT/A, BoNT/B, BoNT/C1, | BoNT/A, BoNT/B, BoNT/C1, |
| BoNT/C1, BoNT/E, | | BoNT/E, BoNT/F, BoNT/G, | BoNT/E, BoNT/F, BoNT/G, |
| BoNT/F, BoNT/G, TeNT, | | TeNT, BaNT, or BuNT | TeNT, BaNT, BuNT, or re- |
| BaNT, or BuNT | | | targeting moeity |
| BoNT/A, BoNT/B, | BoNT/E | BoNT/A, BoNT/B, BoNT/C1 | BoNT/A, BoNT/B, BoNT/C1, |
| BoNT/C1, BoNT/D, | | BoNT/D, BoNT/F, BoNT/G, | BoNT/D, BoNT/F, BoNT/G, |
| BoNT/F, BoNT/G, TeNT, | | TeNT, BaNT, or BuNT | TeNT, BaNT, BuNT, or re- |
| BaNT, or BuNT | | | targeting moeity |
| BoNT/A, BoNT/B, | BoNT/F | BoNT/A, BoNT/B, BoNT/C1, | BoNT/A, BoNT/B, BoNT/C1, |
| BoNT/C1, BoNT/D, | | BoNT/D, BoNT/E, BoNT/G, | BoNT/D, BoNT/E, BoNT/G, |
| BoNT/E, BoNT/G, TeNT, | | TeNT, BaNT, or BuNT | TeNT, BaNT, BuNT, or re- |
| BaNT, or BuNT | | | targeting moeity |
| BoNT/A, BoNT/B, | BoNT/G | BoNT/A, BoNT/B, BoNT/C1, | BoNT/A, BoNT/B, BoNT/C1, |
| BoNT/C1, BoNT/D, | | BoNT/D, BoNT/E, BoNT/F, | BoNT/D, BoNT/E, BoNT/F, |
| BoNT/E, BoNT/F, TeNT, | | TeNT, BaNT, or BuNT | TeNT, BaNT, BuNT, or re- |
| BaNT, or BuNT | | | targeting moeity |
| BoNT/A, BoNT/B, | TeNT | BoNT/A, BoNT/B, BoNT/C1, | BoNT/A, BoNT/B, BoNT/C1, |
| BoNT/C1, BoNT/D, | | BoNT/D, BoNT/E, BoNT/F, | BoNT/D, BoNT/E, BoNT/F, |
| BoNT/E, BoNT/F, BoNT/G, | | BoNT/G, BaNT, or BuNT | BoNT/G, BaNT, BuNT, or re- |
| BaNT, or BuNT | | | targeting moeity |
| BoNT/A, BoNT/B, | BaNT | BoNT/A, BoNT/B, BoNT/C1 | BoNT/A, BoNT/B, BoNT/C1, |
| BoNT/C1, BoNT/D, | | BoNT/D, BoNT/E, BoNT/F, | BoNT/D, BoNT/E, BoNT/F, |
| BoNT/E, BoNT/F, BoNT/G, | | BoNT/G, TeNT, or BuNT | BoNT/G, TeNT, BuNT, or re- |
| TeNT, or BuNT | | | targeting moeity |
| BoNT/A, BoNT/B, | BuNT | BoNT/A, BoNT/B, BoNT/C1 | BoNT/A, BoNT/B, BoNT/C1, |
| BoNT/C1, BoNT/D, | | BoNT/D, BoNT/E, BoNT/F | BoNT/D, BoNT/E, BoNT/F, |
| BoNT/E, BoNT/F, BoNT/G, | | BoNT/G, TeNT, or BaNT | BoNT/G, TeNT, BaNT, or re- |
| TeNT, or BaNT | | | targeting moeity |

¹Included in this category is the replacement of the endogenous Clostridial toxin di-chain loop with the indicated exogenous Clostridial toxin di-chain loop; replacement of the endogenous Clostridial toxin di-chain loop protease cleavage site with the indicated exogenous Clostridial toxin di-chain loop protease cleavage site; the addition of an exogenous Clostridial toxin di-chain loop from the indicated Clostridial toxin within the endogenous Clostridial toxin di-chain loop; and the addition of an exogenous Clostridial toxin di-chain loop protease cleavage site from the indicated Clostridial toxin within the endogenous Clostridial toxin di-chain loop.
²Targeting moeities suitable as binding domains disclosed in the present specification are described in Steward, supra, International Patent Publication No. 2006/008956; Steward, supra, U.S. Patent Application No. 11/776,043; Steward, supra, International Patent Publication No. 2006/009831; Steward, supra, U.S. Patent Publication No. 2006/0211619; Steward, supra, U.S. Patent Application No. 11/776,052; Foster, supra, U.S. Pat. No. 5,989,545; Shone, supra, U.S. Pat. No. 6,461,617; Quinn, supra, U.S. Pat. No. 6,632,440; Steward, supra, U.S. Pat. No. 6,843,998; Donovan, supra, U.S. Pat. U.S. Pat. No. 7,138,127; Foster, supra, U.S. Patent Publication 2003/0180289; Dolly, supra, U.S. Pat. No. 7,132,259; Foster, supra, International Patent Publication WO 2005/023309; Steward, supra, U.S. Patent Application No. 11/376,696; Foster, supra, International Patent Publication WO 2006/059093; Foster, supra, International Patent Publication WO 2006/059105; and Steward, supra, U.S. Patent Application No. 11/776,075.

The di-chain loop region of the Clostridial toxin to be modified can be modified to include an exogenous Clostridial toxin di-chain loop protease cleavage site in addition to the naturally-occurring di-chain loop protease cleavage site (Table 3). In this type of modification, both cleavage sites are operably-linked in-frame to a modified Clostridial toxin as a fusion protein and both sites can be cleaved by their respective proteases. As a non-limiting example, a modified BoNT/E can comprise a di-chain loop containing both the naturally-occurring di-chain loop protease cleavage site and a BoNT/A di-chain loop protease cleavage site that can be cleaved by a BoNT/A di-chain loop protease found in C. botulinum serotype A.

The di-chain loop region can also be modified to replace the naturally-occurring di-chain loop region with an exogenous Clostridial toxin di-chain loop region (Table 3). Such a Clostridial toxin di-chain loop region is operably-linked in-frame to a modified Clostridial toxin as a fusion protein. As a non-limiting example, a BoNT/E di-chain loop region (e.g., SEQ ID NO: 15) can be replaced by a BoNT/A di-chain loop region (e.g., SEQ ID NO: 11) that can be cleaved by a BoNT/A di-chain loop protease found in C. botulinum serotype A.

The di-chain loop region can also be modified to replace a naturally-occurring di-chain loop protease cleavage site with an exogenous Clostridial toxin di-chain loop protease cleavage site (Table 3). Such a Clostridial toxin di-chain loop protease cleavage site is operably-linked in-frame to a modified Clostridial toxin as a fusion protein. As a non-limiting example, the R422-K423 scissile bond of a BoNT/E di-chain loop region can be replaced by a K448-A449 scissile bond from a BoNT/A di-chain loop region that can be cleaved by a BoNT/A di-chain loop protease found in C. botulinum serotype A.

The naturally-occurring di-chain loop protease cleavage site can be made inoperable by altering at least the one of the amino acids flanking the peptide bond cleaved by the naturally-occurring protease, i.e., either P₁, P_{1′} or both P₁ and P_{1′}. More extensive alterations can be made, with the proviso that the two cysteine residues of the di-chain loop region remain intact and formation of the disulfide bridge can still be achieved. Non-limiting examples of an amino acid alteration include deletion of an amino acid or replacement of the original amino acid with a different amino acid. These alterations can be made using standard mutagenesis procedures known to a person skilled in the art. In addition, non-limiting examples of mutagensis procedures, as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. These protocols are routine procedures within the scope of one skilled in the art and from the teaching herein.

Thus, in one embodiment, a naturally-occurring di-chain loop protease cleavage site is made inoperable by altering at least one of the amino acids flanking the peptide bond cleaved by a naturally-occurring protease. In aspects of this embodiment, the P₁ amino acid of the di-chain loop protease cleavage site is altered or the P_{1′} amino acid of the di-chain loop protease cleavage site is altered. In other aspects of this embodiment, either K448 or A449 of BoNT/A is altered; either S441 or L442 of BoNT/A is altered; either K441 or A442 of BoNT/B is altered; either G444 or I445 of BoNT/B is altered; either K449 or T450 of BoNT/C1 is altered; either S445 or L446 of BoNT/C1 is altered; either R445 or D446 of BoNT/D is altered; either K442 or N443 of BoNT/D is altered; either R422 or K423 of BoNT/E is altered; either K419 or G420 of BoNT/E is altered; either K423 or S424 of BoNT/E is altered; either K439 or A440 of BoNT/F is altered; either K436 or G437 of BoNT/F is altered; either K446 or S447 of BoNT/G is altered; either T444 or G445 of BoNT/G is altered; either E448 or Q449 of BoNT/G is altered; or either A457 or S458 of TeNT is altered.

In another embodiment, a naturally-occurring di-chain loop protease cleavage site is made inoperable by altering the two amino acids flanking the peptide bond cleaved by a naturally-occurring protease, i.e., P₁ and P_{1′}. In other aspects of this embodiment, both K448 and A449 of BoNT/A are altered; both S441 and L442 of BoNT/A are altered; both K441 and A442 of BoNT/B are altered; both G444 and I445 of BoNT/B are altered; both K449 and T450 of BoNT/C1 are altered; both S445 and L446 of BoNT/C1 are altered; both R445 and D446 of BoNT/D are altered; both K442 and N443 of BoNT/D are altered; both R422 and K423 of BoNT/E are altered; both K419 and G420 of BoNT/E are altered; both K423 and S424 of BoNT/E are altered; both K439 and A440 of BoNT/F are altered; both K436 and G437 of BoNT/F are altered; both K446 and S447 of BoNT/G are altered; both T444 and G445 of BoNT/G are altered; both E448 and Q449 of BoNT/G are altered; or both A457 and S458 of TeNT are altered.

In other aspects of this embodiment, a naturally-occurring di-chain loop protease cleavage site is made inoperable by altering, e.g., at least two amino acids within the di-chain loop region; at least three amino acids within the di-chain loop region; at least four amino acids within the di-chain loop region; at least five amino acids within the di-chain loop region; at least six amino acids within the di-chain loop region; at least seven amino acids within the di-chain loop region; at least eight amino acids within the di-chain loop region; at least nine amino acids within the di-chain loop region; at least ten amino acids within the di-chain loop region; or at least 15 amino acids within the di-chain loop region. In still other aspects of this embodiment, a naturally-occurring di-chain loop protease cleavage site is made inoperable by altering one of the amino acids flanking the peptide bond cleaved by a naturally-occurring protease and, e.g., at least one more amino acid within the di-chain loop region; at least two more amino acids within the di-chain loop region; at least three more amino acids within the di-chain loop region; at least four more amino acids within the di-chain loop region; at least five more amino acids within the di-chain loop region; at least six more amino acids within the di-chain loop region; at least seven more amino acids within the di-chain loop region; at least eight more amino acids within the di-chain loop region; at least nine more amino acids within the di-chain loop region; at least ten more amino acids within the di-chain loop region; at least 15 more amino acids within the di-chain loop region. In yet other aspects of this embodiment, a naturally-occurring di-chain loop protease cleavage site is made inoperable by altering the two amino acids flanking the peptide bond cleaved by a naturally-occurring protease and, e.g., at least one more amino acid within the di-chain loop region; at least two more amino acids within the di-chain loop region; at least three more amino acids within the di-chain loop region; at least four more amino acids within the di-chain loop region; at least five more amino acids within the di-chain loop region; at least six more amino acids within the di-chain loop region; at least seven more amino acids within the di-chain loop region; at least eight more amino acids within the di-chain loop region; at least nine more amino acids within the di-chain loop region; at least ten more amino acids within the di-chain loop region; at least 15 more amino acids within the di-chain loop region.

In other aspects of this embodiment, a naturally-occurring di-chain loop protease cleavage site is made inoperable by altering, e.g., at most two amino acids within the di-chain loop region; at most three amino acids within the di-chain loop region; at most four amino acids within the di-chain loop region; at most five amino acids within the di-chain loop region; at most six amino acids within the di-chain loop region; at most seven amino acids within the di-chain loop region; at most eight amino acids within the di-chain loop region; at most nine amino acids within the di-chain loop region; at most ten amino acids within the di-chain loop region; or at most 15 amino acids within the di-chain loop region. In still other aspects of this embodiment, a naturally-occurring di-chain loop protease cleavage site is made inoperable by altering one of the amino acids flanking the peptide bond cleaved by a naturally-occurring protease and, e.g., at most one more amino acid within the di-chain loop region; at most two more amino acids within the di-chain loop region; at most three more amino acids within the di-chain loop region; at most four more amino acids within the di-chain loop region; at most five more amino acids within the di-chain loop region; at most six more amino acids within the di-chain loop region; at most seven more amino acids within the di-chain loop region; at most eight more amino acids within the di-chain loop region; at most nine more amino acids within the di-chain loop region; at most ten more amino acids within the di-chain loop region; at most 15 more amino acids within the di-chain loop region. In yet other aspects of this embodiment, a naturally-occurring di-chain loop protease cleavage site is made inoperable by altering the two amino acids flanking the peptide bond cleaved by a naturally-occurring protease and, e.g., at most one more amino acid within the di-chain loop region; at most two more amino acids within the di-chain loop region; at most three more amino acids within the di-chain loop region; at most four more amino acids within the di-chain loop region; at most five more amino acids within the di-chain loop region; at most six more amino acids within the di-chain loop region; at most seven more amino acids within the di-chain loop region; at most eight more amino acids within the di-chain loop region; at most nine more amino acids within the di-chain loop region; at most ten more amino acids within the di-chain loop region; at most 15 more amino acids within the di-chain loop region.

It is envisioned that the di-chain loop region of a Clostridial toxin can be modified to include any of the other Clostridial toxin di-chain loop regions. In aspects of this embodiment, a Clostridial toxin di-chain loop region can be modified to comprise, e.g., a BoNT/A di-chain loop region, a BoNT/B di-chain loop region, a BoNT/C1 di-chain loop region, a BoNT/D di-chain loop region, a BoNT/E di-chain loop region, a BoNT/F di-chain loop region, a BoNT/G di-chain loop region, a TeNT di-chain loop region, a BaNT di-chain loop region or a BuNT di-chain loop region. In other aspects of this embodiment, an exogenous Clostridial toxin di-chain loop region, in addition to the naturally-occurring protease cleavage site, can be modified to comprise, e.g., a BoNT/A di-chain loop region, a BoNT/B di-chain loop region, a BoNT/C1 di-chain loop region, a BoNT/D di-chain loop region, a BoNT/E di-chain loop region, a BoNT/F di-chain loop region, a BoNT/G di-chain loop region, a TeNT di-chain loop region, a BaNT di-chain loop region or a BuNT di-chain loop region.

In still other aspects of this embodiment, a di-chain loop of a Clostridial toxin can be modified to replace a naturally-occurring protease cleavage site with, e.g., a BoNT/A substrate cleavage site, a BoNT/B substrate cleavage site, a BoNT/C1 substrate cleavage site, a BoNT/D substrate cleavage site, a BoNT/E substrate cleavage site, a BoNT/F substrate cleavage site, a BoNT/G substrate cleavage site, a TeNT substrate cleavage site, a BaNT substrate cleavage site or a BuNT substrate cleavage site.

The location of the Clostridial toxin substrate cleavage site can be anywhere in the Clostridial toxin, with the proviso that cleavage of the site must occur between the two cysteine residues that form the single disulfide bridge of toxin. Thus, in aspects of this embodiment, location of a Clostridial toxin substrate cleavage site can be, e.g., anywhere in the BoNT/A of SEQ ID NO: 1, with the proviso that cleavage occurs between cysteine 430 and cysteine 454; anywhere in the BoNT/B of SEQ ID NO: 2, with the proviso that cleavage occurs between cysteine 437 and cysteine 446; anywhere in the BoNT/C1 of SEQ ID NO: 2, with the proviso that cleavage occurs between cysteine 437 and cysteine 453; anywhere in the BoNT/D of SEQ ID NO: 4, with the proviso that cleavage occurs between cysteine 437 and cysteine 450; anywhere in the BoNT/E of SEQ ID NO: 5, with the proviso that cleavage occurs between cysteine 412 and cysteine 426; anywhere in the BoNT/F of SEQ ID NO: 6, with the proviso that cleavage occurs between cysteine 429 and cysteine 445; anywhere in the BoNT/G of SEQ ID NO: 7, with the proviso that cleavage occurs between cysteine 436 and cysteine 450; or anywhere in the TeNT of SEQ ID NO: 8, with the proviso that cleavage occurs between cysteine 439 and cysteine 467.

It is understood that a modified Clostridial toxin disclosed in the present specification can optionally include one or more additional components. As a non-limiting example of an optional component, a modified Clostridial toxin can further comprise a flexible region comprising a flexible spacer. Non-limiting examples of a flexible spacer include, e.g., a G-spacer GGGGS (SEQ ID NO: 21) or an A-spacer EAAAK (SEQ ID NO: 22). A flexible region comprising flexible spacers can be used to adjust the length of a polypeptide region in order to optimize a characteristic, attribute or property of a polypeptide. Such a flexible region is operably-linked in-frame to the modified Clostridial toxin as a fusion protein. As a non-limiting example, a polypeptide region comprising one or more flexible spacers in tandem can be use to better expose a protease cleavage site thereby facilitating cleavage of that site by a protease. As another non-limiting example, a polypeptide region comprising one or more flexible spacers in tandem can be use to better present a ligand domain, thereby facilitating the binding of that ligand domain to its binding domain on a receptor.

Thus, in an embodiment, a modified Clostridial toxin disclosed in the present specification can further comprise a flexible region comprising a flexible spacer. In another embodiment, a modified Clostridial toxin disclosed in the present specification can further comprise flexible region comprising a plurality of flexible spacers in tandem. In aspects of this embodiment, a flexible region can comprise in tandem, e.g., at least 1 G-spacer, at least 2 G-spacers, at least 3 G-spacers, at least 4 G-spacers or at least 5 G-spacers. In other aspects of this embodiment, a flexible region can comprise in tandem, e.g., at most 1 G-spacer, at most 2 G-spacers, at most 3 G-spacers, at most 4 G-spacers or at most 5 G-spacers. In still other aspects of this embodiment, a flexible region can comprise in tandem, e.g., at least 1 A-spacer, at least 2 A-spacers, at least 3 A-spacers, at least 4 A-spacers or at least 5 A-spacers. In still other aspects of this embodiment, a flexible region can comprise in tandem, e.g., at most 1 A-spacer, at most 2 A-spacers, at most 3 A-spacers, at most 4 A-spacers or at most 5 A-spacers. In another aspect of this embodiment, a modified Clostridial toxin can comprise a flexible region comprising one or more copies of the same flexible spacers, one or more copies of different flexible-spacer regions, or any combination thereof.

As another non-limiting example of an optional component, a modified Clostridial toxin can further comprise an epitope-binding region. An epitope-binding region can be used in a wide variety of procedures involving, e.g., protein purification and protein visualization. Such an epitope-binding region is operably-linked in-frame to a modified Clostridial toxin as a fusion protein. Non-limiting examples of an epitope-binding region include, e.g., FLAG, Express™ (SEQ ID NO: 23), human Influenza virus hemagluttinin (HA) (SEQ ID NO: 24), human p62^{c-Myc} protein (c-MYC) (SEQ ID NO: 25), Vesicular Stomatitis Virus Glycoprotein (VSV-G) (SEQ ID NO: 26), Substance P (SEQ ID NO: 27), glycoprotein-D precursor of Herpes simplex virus (HSV) (SEQ ID NO: 28), V5 (SEQ ID NO: 29), AU1 (SEQ ID NO: 30) and AU5 (SEQ ID NO: 31); affinity-binding, such as. e.g., polyhistidine (HIS) (SEQ ID NO: 32), streptavidin binding peptide (strep), and biotin or a biotinylation sequence; peptide-binding regions, such as. e.g., the glutathione binding domain of glutathione-S-transferase, the calmodulin binding domain of the calmodulin binding protein, and the maltose binding domain of the maltose binding protein. Non-limiting examples of specific protocols for selecting, making and using an appropriate binding peptide are described in, e.g., Epitope Tagging, pp. 17.90-17.93 (Sambrook and Russell, eds., MOLECULAR CLONING A LABORATORY MANUAL, Vol. 3, 3^{rd} ed. 2001); ANTIBODIES: A LABORATORY MANUAL (Edward Harlow & David Lane, eds., Cold Spring Harbor Laboratory Press, 2^{nd} ed. 1998); and USING ANTIBODIES: A LABORATORY MANUAL: PORTABLE PROTOCOL No. I (Edward Harlow & David Lane, Cold Spring Harbor Laboratory Press, 1998). In addition, non-limiting examples of binding peptides as well as well-characterized reagents, conditions and protocols are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; and Stratagene, La Jolla, Calif. These protocols are routine procedures well within the scope of one skilled in the art and from the teaching herein.

Thus, in an embodiment, a modified Clostridial toxin disclosed in the present specification can further comprise an epitope-binding region. In another embodiment, a modified Clostridial toxin disclosed in the present specification can further comprises a plurality of epitope-binding regions. In aspects of this embodiment, a modified Clostridial toxin can comprise, e.g., at least 1 epitope-binding region, at least 2 epitope-binding regions, at least 3 epitope-binding regions, at least 4 epitope-binding regions or at least 5 epitope-binding regions. In other aspects of this embodiment, a modified Clostridial toxin can comprise, e.g., at most 1 epitope-binding region, at most 2 epitope-binding regions, at most 3 epitope-binding regions, at most 4 epitope-binding regions or at most 5 epitope-binding regions. In another aspect of this embodiment, a modified Clostridial toxin can comprise one or more copies of the same epitope-binding region, one or more copies of different epitope-binding regions, or any combination thereof.

The location of an epitope-binding region can be in various positions, including, without limitation, at the amino terminus of a modified Clostridial toxin, within a modified Clostridial toxin, or at the carboxyl terminus of a modified Clostridial toxin. Thus, in an embodiment, an epitope-binding region is located at the amino-terminus of a modified Clostridial toxin. In such a location, a start methionine should be placed in front of the epitope-binding region. In addition, it is known in the art that when adding a polypeptide that is operationally-linked to the amino terminus of another polypeptide comprising the start methionine that the original methionine residue can be deleted. This is due to the fact that the added polypeptide will contain a new start methionine and that the original start methionine may reduce optimal expression of the fusion protein. In aspects of this embodiment, an epitope-binding region located at the amino-terminus of a modified Clostridial toxin disclosed in the present specification can be, e.g., a FLAG, Express™ epitope-binding region, a human Influenza virus hemagluttinin (HA) epitope-binding region, a human p62^{c-Myc} protein (c-MYC) epitope-binding region, a Vesicular Stomatitis Virus Glycoprotein (VSV-G) epitope-binding region, a Substance P epitope-binding region, a glycoprotein-D precursor of Herpes simplex virus (HSV) epitope-binding region, a V5 epitope-binding region, a AU1 epitope-binding region, a AU5 epitope-binding region, a polyhistidine epitope-binding region, a streptavidin binding peptide epitope-binding region, a biotin epitope-binding region, a biotinylation epitope-binding region, a glutathione binding domain of glutathione-S-transferase, a calmodulin binding domain of the calmodulin binding protein or a maltose binding domain of the maltose binding protein.

In another embodiment, an epitope-binding region is located at the carboxyl-terminus of a modified Clostridial toxin. In aspects of this embodiment, an epitope-binding region located at the carboxyl-terminus of a modified Clostridial toxin disclosed in the present specification can be, e.g., a FLAG, Express™ epitope-binding region, a human Influenza virus hemagluttinin (HA) epitope-binding region, a human p62^{c-Myc} protein (c-MYC) epitope-binding region, a Vesicular Stomatitis Virus Glycoprotein (VSV-G) epitope-binding region, a Substance P epitope-binding region, a glycoprotein-D precursor of Herpes simplex virus (HSV) epitope-binding region, a V5 epitope-binding region, a AU1 epitope-binding region, a AU5 epitope-binding region, a polyhistidine epitope-binding region, a streptavidin binding peptide epitope-binding region, a biotin epitope-binding region, a biotinylation epitope-binding region, a glutathione binding domain of glutathione-S-transferase, a calmodulin binding domain of the calmodulin binding protein or a maltose binding domain of the maltose binding protein.

Aspects of the present invention provide, in part modified Clostridial toxins. As used herein, the term “modified Clostridial toxin” means any naturally-occurring Clostridial toxin or non-naturally occurring Clostridial toxin comprising at least 1) the replacement of a naturally-occurring di-chain loop protease cleavage site with a di-chain loop protease cleavage site from another Clostridial toxin, 2) the addition of a Clostridial toxin di-chain loop protease cleavage site as disclosed in the present specification into the di-chain loop region of a naturally-occurring Clostridial toxin, 3) the replacement of a naturally-occurring di-chain loop region with a di-chain loop region from another Clostridial toxin, or 4) the addition of a Clostridial toxin di-chain loop region as disclosed in the present specification into the di-chain loop region of a naturally-occurring Clostridial toxin.

It is understood that all such modifications do not substantially affect the ability of a Clostridial toxin to intoxicate a cell. As used herein, the term “do not substantially affect” means a Clostridial toxin can still execute the overall cellular mechanism whereby a Clostridial toxin enters a neuron and inhibits neurotransmitter release and encompasses the binding of a Clostridial toxin to a low or high affinity receptor complex, the internalization of the toxin/receptor complex, the translocation of the Clostridial toxin light chain into the cytoplasm and the enzymatic modification of a Clostridial toxin substrate. In aspects of this embodiment, the modified Clostridial toxin is, e.g., at least 10% as toxic as a naturally-occurring Clostridial toxin, at least 20% as toxic as a naturally-occurring Clostridial toxin, at least 30% as toxic as a naturally-occurring Clostridial toxin, at least 40% as toxic as a naturally-occurring Clostridial toxin, at least 50% as toxic as a naturally-occurring Clostridial toxin, at least 60% as toxic as a naturally-occurring Clostridial toxin, at least 70% as toxic as a naturally-occurring Clostridial toxin, at least 80% as toxic as a naturally-occurring Clostridial toxin, at least 90% as toxic as a naturally-occurring Clostridial toxin or at least 95% as toxic as a naturally-occurring Clostridial toxin. In aspects of this embodiment, the modified Clostridial toxin is, e.g., at most 10% as toxic as a naturally-occurring Clostridial toxin, at most 20% as toxic as a naturally-occurring Clostridial toxin, at most 30% as toxic as a naturally-occurring Clostridial toxin, at most 40% as toxic as a naturally-occurring Clostridial toxin, at most 50% as toxic as a naturally-occurring Clostridial toxin, at most 60% as toxic as a naturally-occurring Clostridial toxin, at most 70% as toxic as a naturally-occurring Clostridial toxin, at most 80% as toxic as a naturally-occurring Clostridial toxin, at most 90% as toxic as a naturally-occurring Clostridial toxin or at most 95% as toxic as a naturally-occurring Clostridial toxin.

Aspects of the present invention provide, in part, polynucleotide molecules. As used herein, the term “polynucleotide molecule” is synonymous with “nucleic acid molecule” and means a polymeric form of nucleotides, such as, e.g., ribonucleotides and deoxyribonucleotides, of any length. It is envisioned that any and all polynucleotide molecules that can encode a modified Clostridial toxin disclosed in the present specification can be useful, including, without limitation naturally-occurring and non-naturally-occurring DNA molecules and naturally-occurring and non-naturally-occurring RNA molecules. Non-limiting examples of naturally-occurring and non-naturally-occurring DNA molecules include single-stranded DNA molecules, double-stranded DNA molecules, genomic DNA molecules, cDNA molecules, vector constructs, such as, e.g., plasmid constructs, phagmid constructs, bacteriophage constructs, retroviral constructs and artificial chromosome constructs. Non-limiting examples of naturally-occurring and non-naturally-occurring RNA molecules include single-stranded RNA, double stranded RNA and mRNA.

Well-established molecular biology techniques that may be necessary to make a polynucleotide molecule encoding a modified Clostridial toxin disclosed in the present specification including, but not limited to, procedures involving polymerase chain reaction (PCR) amplification, restriction enzyme reactions, agarose gel electrophoresis, nucleic acid ligation, bacterial transformation, nucleic acid purification, nucleic acid sequencing and recombination-based techniques are routine procedures well within the scope of one skilled in the art and from the teaching herein. Non-limiting examples of specific protocols necessary to make a polynucleotide molecule encoding a modified Clostridial toxin are described in e.g., MOLECULAR CLONING A LABORATORY MANUAL, supra, (2001); and CURRENT PROTOCOLS IN MOLECULAR BIOLOGY (Frederick M. Ausubel et al., eds. John Wiley & Sons, 2004). Additionally, a variety of commercially available products useful for making a polynucleotide molecule encoding a modified Clostridial toxin are widely available. These protocols are routine procedures well within the scope of one skilled in the art and from the teaching herein.

Another aspect of the present invention provides a method of producing a modified Clostridial toxin comprising an exogenous Clostridial toxin di-chain loop region including a Clostridial toxin di-chain loop protease cleavage site from a different Clostridial toxin, such method comprising the step of expressing a polynucleotide molecule encoding a modified Clostridial toxin in a cell. Another aspect of the present invention provides a method of producing a modified Clostridial toxin comprising an exogenous Clostridial toxin di-chain loop region including a Clostridial toxin di-chain loop protease cleavage site from a different Clostridial toxin, such method comprising the steps of introducing an expression construct comprising a polynucleotide molecule encoding the modified Clostridial toxin into a cell and expressing the expression construct in the cell.

The methods disclosed in the present specification include, in part, a modified Clostridial toxin. It is envisioned that any and all modified Clostridial toxins disclosed in the present specification can be produced using the methods disclosed in the present specification. Thus, aspects of this embodiment include producing, without limitation, naturally occurring Clostridial toxins, naturally occurring Clostridial toxins variants, such as, e.g., Clostridial toxins isoforms and Clostridial toxins subtypes, non-naturally occurring Clostridial toxins variants, such as, e.g., conservative Clostridial toxins variants, non-conservative Clostridial toxins variants and Clostridial toxins fragments thereof, or any combination thereof.

The methods disclosed in the present specification include, in part, a polynucleotide molecule. It is envisioned that any and all polynucleotide molecules disclosed in the present specification can be used. Thus, aspects of this embodiment include, without limitation, naturally-occurring and non-naturally-occurring DNA molecules include single-stranded DNA molecules, double-stranded DNA molecules, genomic DNA molecules, cDNA molecules, vector constructs, such as, e.g., plasmid constructs, phagmid constructs, bacteriophage constructs, retroviral constructs and artificial chromosome constructs. Non-limiting examples of naturally-occurring and non-naturally-occurring RNA molecules include single-stranded RNA, double stranded RNA and mRNA.

The methods disclosed in the present specification include, in part, an expression construct. An expression construct comprises a polynucleotide molecule disclosed in the present specification operably-linked to an expression vector useful for expressing the polynucleotide molecule in a cell or cell-free extract. A wide variety of expression vectors can be employed for expressing a polynucleotide molecule encoding a modified Clostridial toxin, including, without limitation, a viral expression vector; a prokaryotic expression vector; eukaryotic expression vectors, such as, e.g., a yeast expression vector, an insect expression vector and a mammalian expression vector; and a cell-free extract expression vector. It is further understood that expression vectors useful to practice aspects of these methods may include those which express a modified Clostridial toxin under control of a constitutive, tissue-specific, cell-specific or inducible promoter element, enhancer element or both. Non-limiting examples of expression vectors, along with well-established reagents and conditions for making and using an expression construct from such expression vectors are readily available from commercial vendors that include, without limitation, BD Biosciences-Clontech, Palo Alto, Calif.; BD Biosciences Pharmingen, San Diego, Calif.; Invitrogen, Inc, Carlsbad, Calif.; EMD Biosciences-Novagen, Madison, Wis.; QIAGEN, Inc., Valencia, Calif.; and Stratagene, La Jolla, Calif. The selection, making and use of an appropriate expression vector are routine procedures well within the scope of one skilled in the art and from the teachings herein.

Thus, aspects of this embodiment include, without limitation, a viral expression vector operably-linked to a polynucleotide molecule encoding a modified Clostridial toxin; a prokaryotic expression vector operably-linked to a polynucleotide molecule encoding a modified Clostridial toxin; a yeast expression vector operably-linked to a polynucleotide molecule encoding a modified Clostridial toxin; an insect expression vector operably-linked to a polynucleotide molecule encoding a modified Clostridial toxin; and a mammalian expression vector operably-linked to a polynucleotide molecule encoding a modified Clostridial toxin. Other aspects of this embodiment include, without limitation, expression constructs suitable for expressing a modified Clostridial toxin disclosed in the present specification using a cell-free extract comprising a cell-free extract expression vector operably linked to a polynucleotide molecule encoding a modified Clostridial toxin.

The methods disclosed in the present specification include, in part, a cell. It is envisioned that any and all cells can be used. Thus, aspects of this embodiment include, without limitation, prokaryotic cells including, without limitation, strains of aerobic, microaerophilic, capnophilic, facultative, anaerobic, gram-negative and gram-positive bacterial cells such as those derived from, e.g., Escherichia coli, Bacillus subtilis, Bacillus licheniformis, Bacteroides fragilis, Clostridia perfringens, Clostridia difficile, Caulobacter crescentus, Lactococcus lactis, Methylobacterium extorquens, Neisseria meningirulls, Neisseria meningitidis, Pseudomonas fluorescens and Salmonella typhimurium; and eukaryotic cells including, without limitation, yeast strains, such as, e.g., those derived from Pichia pastoris, Pichia methanolica, Pichia angusta, Schizosaccharomyces pombe, Saccharomyces cerevisiae and Yarrowia lipolytica; insect cells and cell lines derived from insects, such as, e.g., those derived from Spodoptera frugiperda, Trichoplusia ni, Drosophila melanogaster and Manduca sexta; and mammalian cells and cell lines derived from mammalian cells, such as, e.g., those derived from mouse, rat, hamster, porcine, bovine, equine, primate and human. Cell lines may be obtained from the American Type Culture Collection (2004); European Collection of Cell Cultures (2204); and the German Collection of Microorganisms and Cell Cultures (2004). Non-limiting examples of specific protocols for selecting, making and using an appropriate cell line are described in e.g., INSECT CELL CULTURE ENGINEERING (Mattheus F. A. Goosen et al. eds., Marcel Dekker, 1993); INSECT CELL CULTURES: FUNDAMENTAL AND APPLIED ASPECTS (J. M. Vlak et al. eds., Kluwer Academic Publishers, 1996); Maureen A. Harrison & Ian F. Rae, GENERAL TECHNIQUES OF CELL CULTURE (Cambridge University Press, 1997); CELL AND TISSUE CULTURE: LABORATORY PROCEDURES (Alan Doyle et al eds., John Wiley and Sons, 1998); R. Ian Freshney, CULTURE OF ANIMAL CELLS: A MANUAL OF BASIC TECHNIQUE (Wiley-Liss, 4^{th} ed. 2000); ANIMAL CELL CULTURE: A PRACTICAL APPROACH (John R. W. Masters ed., Oxford University Press, 3^{rd} ed. 2000); MOLECULAR CLONING A LABORATORY MANUAL, supra, (2001); BASIC CELL CULTURE: A PRACTICAL APPROACH (John M. Davis, Oxford Press, 2^{nd} ed. 2002); and CURRENT PROTOCOLS IN MOLECULAR BIOLOGY, supra, (2004). These protocols are routine procedures within the scope of one skilled in the art and from the teaching herein.

The methods disclosed in the present specification include, in part, introducing into a cell a polynucleotide molecule. A polynucleotide molecule introduced into a cell can be transiently or stably maintained by that cell. Stably-maintained polynucleotide molecules may be extra-chromosomal and replicate autonomously, or they may be integrated into the chromosomal material of the cell and replicate non-autonomously. It is envisioned that any and all methods for introducing a polynucleotide molecule disclosed in the present specification into a cell can be used. Methods useful for introducing a nucleic acid molecule into a cell include, without limitation, chemical-mediated transfection such as, e.g., calcium phosphate-mediated, diethyl-aminoethyl (DEAE) dextran-mediated, lipid-mediated, polyethyleneimine (PEI)-mediated, polylysine-mediated and polybrene-mediated; physical-mediated transfection, such as, e.g., biolistic particle delivery, microinjection, protoplast fusion and electroporation; and viral-mediated transfection, such as, e.g., retroviral-mediated transfection, see, e.g., Introducing Cloned Genes into Cultured Mammalian Cells, pp. 16.1-16.62 (Sambrook & Russell, eds., Molecular Cloning A Laboratory Manual, Vol. 3, 3^{rd} ed. 2001). One skilled in the art understands that selection of a specific method to introduce an expression construct into a cell will depend, in part, on whether the cell will transiently contain an expression construct or whether the cell will stably contain an expression construct. These protocols are routine procedures within the scope of one skilled in the art and from the teaching herein.

In an aspect of this embodiment, a chemical-mediated method, termed transfection, is used to introduce a polynucleotide molecule encoding a modified Clostridial toxin into a cell. In chemical-mediated methods of transfection the chemical reagent forms a complex with the nucleic acid that facilitates its uptake into the cells. Such chemical reagents include, without limitation, calcium phosphate-mediated, see, e.g., Martin Jordan & Florian Worm, Transfection of Adherent and Suspended Cells by Calcium Phosphate, 33(2) Methods 136-143 (2004); diethyl-aminoethyl (DEAE) dextran-mediated, lipid-mediated, cationic polymer-mediated like polyethyleneimine (PEI)-mediated and polylysine-mediated and polybrene-mediated, see, e.g., Chun Zhang et al., Polyethylenimine Strategies for Plasmid Delivery to Brain-Derived Cells, 33(2) Methods 144-150 (2004). Such chemical-mediated delivery systems can be prepared by standard methods and are commercially available, see, e.g., CellPhect Transfection Kit (Amersham Biosciences, Piscataway, N.J.); Mammalian Transfection Kit, Calcium phosphate and DEAE Dextran, (Stratagene, Inc., La Jolla, Calif.); Lipofectamine™ Transfection Reagent (Invitrogen, Inc., Carlsbad, Calif.); ExGen 500 Transfection kit (Fermentas, Inc., Hanover, Md.), and SuperFect and Effectene Transfection Kits (Qiagen, Inc., Valencia, Calif.).

In another aspect of this embodiment, a physical-mediated method is used to introduce a polynucleotide molecule encoding a modified Clostridial toxin into a cell. Physical techniques include, without limitation, electroporation, biolistic and microinjection. Biolistics and microinjection techniques perforate the cell wall in order to introduce the nucleic acid molecule into the cell, see, e.g., Jeike E. Biewenga et al., Plasmid-Mediated Gene Transfer in Neurons Using the Biolistics Technique, 71(1) J. Neurosci. Methods. 67-75 (1997); and John O'Brien & Sarah C. R. Lummis, Biolistic and Diolistic Transfection: Using the Gene Gun to Deliver DNA and Lipophilic Dyes into Mammalian Cells, 33(2) Methods 121-125 (2004). Electroporation, also termed electropermeabilization, uses brief, high-voltage, electrical pulses to create transient pores in the membrane through which the nucleic acid molecules enter and can be used effectively for stable and transient transfections of all cell types, see, e.g., M. Golzio et al., In vitro and in vivo Electric Field-Mediated Permeabilization, Gene Transfer, and Expression, 33(2) Methods 126-135 (2004); and Oliver Gresch et al., New Non-Viral Method for Gene Transfer into Primary Cells, 33(2) Methods 151-163 (2004).

In another aspect of this embodiment, a viral-mediated method, termed transduction, is used to introduce a polynucleotide molecule encoding a modified Clostridial toxin into a cell. In viral-mediated methods of transient transduction, the process by which viral particles infect and replicate in a host cell has been manipulated in order to use this mechanism to introduce a nucleic acid molecule into the cell. Viral-mediated methods have been developed from a wide variety of viruses including, without limitation, retroviruses, adenoviruses, adeno-associated viruses, herpes simplex viruses, picornaviruses, alphaviruses and baculoviruses, see, e.g., Armin Blesch, Lentiviral and MLV based Retroviral Vectors for ex vivo and in vivo Gene Transfer, 33(2) Methods 164-172 (2004); and Maurizio Federico, From Lentiviruses to Lentivirus Vectors, 229 Methods Mol. Biol. 3-15 (2003); E. M. Poeschla, Non-Primate Lentiviral Vectors, 5(5) Curr. Opin. Mol. Ther. 529-540 (2003); Karim Benihoud et al, Adenovirus Vectors for Gene Delivery, 10(5) Curr. Opin. Biotechnol. 440-447 (1999); H. Bueler, Adeno-Associated Viral Vectors for Gene Transfer and Gene Therapy, 380(6) Biol. Chem. 613-622 (1999); Chooi M. Lai et al., Adenovirus and Adeno-Associated Virus Vectors, 21(12) DNA Cell Biol. 895-913 (2002); Edward A. Burton et al., Gene Delivery Using Herpes Simplex Virus Vectors, 21(12) DNA Cell Biol. 915-936 (2002); Paola Grandi et al., Targeting HSV Amplicon Vectors, 33(2) Methods 179-186 (2004); Ilya Frolov et al., Alphavirus-Based Expression Vectors: Strategies and Applications, 93(21) Proc. Natl. Acad. Sci. U.S.A. 11371-11377 (1996); Markus U. Ehrengruber, Alphaviral Gene Transfer in Neurobiology, 59(1) Brain Res. Bull. 13-22 (2002); Thomas A. Kost & J. Patrick Condreay, Recombinant Baculoviruses as Mammalian Cell Gene-Delivery Vectors, 20(4) Trends Biotechnol. 173-180 (2002); and A. Huser & C. Hofmann, Baculovirus Vectors Novel Mammalian Cell Gene-Delivery Vehicles and Their Applications, 3(1) Am. J. Pharmacogenomics 53-63 (2003).

Adenoviruses, which are non-enveloped, double-stranded DNA viruses, are often selected for mammalian cell transduction because adenoviruses handle relatively large polynucleotide molecules of about 36 kb, are produced at high titer, and can efficiently infect a wide variety of both dividing and non-dividing cells, see, e.g., Wim T. J. M. C. Hermens et al., Transient Gene Transfer to Neurons and Glia: Analysis of Adenoviral Vector Performance in the CNS and PNS, 71(1) J. Neurosci. Methods 85-98 (1997); and Hiroyuki Mizuguchi et al., Approaches for Generating Recombinant Adenovirus Vectors, 52(3) Adv. Drug Deliv. Rev. 165-176 (2001). Transduction using adenoviral-based system do not support prolonged protein expression because the nucleic acid molecule is carried from an episome in the cell nucleus, rather than being integrated into the host cell chromosome. Adenoviral vector systems and specific protocols for how to use such vectors are disclosed in, e.g., ViraPower™ Adenoviral Expression System (Invitrogen, Inc., Carlsbad, Calif.) and ViraPower™ Adenoviral Expression System Instruction Manual 25-0543 version A, Invitrogen, Inc., (Jul. 15, 2002); and AdEasy™ Adenoviral Vector System (Stratagene, Inc., La Jolla, Calif.) and AdEasy™ Adenoviral Vector System Instruction Manual 064004f, Stratagene, Inc.

Polynucleotide molecule delivery can also use single-stranded RNA retroviruses, such as, e.g., oncoretroviruses and lentiviruses. Retroviral-mediated transduction often produce transduction efficiencies close to 100%, can easily control the proviral copy number by varying the multiplicity of infection (MOI), and can be used to either transiently or stably transduce cells, see, e.g., Tiziana Tonini et al., Transient Production Of Retroviral-and Lentiviral-Based Vectors For the Transduction of Mammalian Cells, 285 Methods Mol. Biol. 141-148 (2004); Armin Blesch, Lentiviral and MLV Based Retroviral Vectors for ex vivo and in vivo Gene Transfer, 33(2) Methods 164-172 (2004); Felix Recillas-Targa, Gene Transfer and Expression in Mammalian Cell Lines and Transgenic Animals, 267 Methods Mol. Biol. 417-433 (2004); and Roland Wolkowicz et al., Lentiviral Vectors for the Delivery of DNA into Mammalian Cells, 246 Methods Mol. Biol. 391-411 (2004). Retroviral particles consist of an RNA genome packaged in a protein capsid, surrounded by a lipid envelope. The retrovirus infects a host cell by injecting its RNA into the cytoplasm along with the reverse transcriptase enzyme. The RNA template is then reverse transcribed into a linear, double stranded cDNA that replicates itself by integrating into the host cell genome. Viral particles are spread both vertically (from parent cell to daughter cells via the provirus) as well as horizontally (from cell to cell via virions). This replication strategy enables long-term persistent expression since the nucleic acid molecules of interest are stably integrated into a chromosome of the host cell, thereby enabling long-term expression of the protein. For instance, animal studies have shown that lentiviral vectors injected into a variety of tissues produced sustained protein expression for more than 1 year, see, e.g., Luigi Naldini et al., In vivo Gene Delivery and Stable Transduction of Non-Dividing Cells By a Lentiviral Vector, 272(5259) Science 263-267 (1996). The Oncoretroviruses-derived vector systems, such as, e.g., Moloney murine leukemia virus (MoMLV), are widely used and infect many different non-dividing cells. Lentiviruses can also infect many different cell types, including dividing and non-dividing cells and possess complex envelope proteins, which allows for highly specific cellular targeting.

Retroviral vectors and specific protocols for how to use such vectors are disclosed in, e.g., U.S. patent Nos. Manfred Gossen & Hermann Bujard, Tight Control of Gene Expression in Eukaryotic Cells By Tetracycline-Responsive Promoters, U.S. Pat. No. 5,464,758 (Nov. 7, 1995) and Hermann Bujard & Manfred Gossen, Methods for Regulating Gene Expression, U.S. Pat. No. 5,814,618 (Sep. 29, 1998) David S. Hogness, Polynucleotides Encoding Insect Steroid Hormone Receptor Polypeptides and Cells Transformed With Same, U.S. Pat. No. 5,514,578 (May 7, 1996) and David S. Hogness, Polynucleotide Encoding Insect Ecdysone Receptor, U.S. Pat. No. 6,245,531 (Jun. 12, 2001); Elisabetta Vegeto et al., Progesterone Receptor Having C. Terminal Hormone Binding Domain Truncations, U.S. Pat. No. 5,364,791 (Nov. 15, 1994), Elisabetta Vegeto et al., Mutated Steroid Hormone Receptors, Methods For Their Use and Molecular Switch For Gene Therapy, U.S. Pat. No. 5,874,534 (Feb. 23, 1999) and Elisabetta Vegeto et al., Mutated Steroid Hormone Receptors, Methods For Their Use and Molecular Switch For Gene Therapy, U.S. Pat. No. 5,935,934 (Aug. 10, 1999). Furthermore, such viral delivery systems can be prepared by standard methods and are commercially available, see, e.g., BD™ Tet-Off and Tet-On Gene Expression Systems (BD Biosciences-Clonetech, Palo Alto, Calif.) and BD™ Tet-Off and Tet-On Gene Expression Systems User Manual, PT3001-1, BD Biosciences Clonetech, (Mar. 14, 2003), GeneSwitch™ System (Invitrogen, Inc., Carlsbad, Calif.) and GeneSwitch™ System A Mifepristone-Regulated Expression System for Mammalian Cells version D, 25-0313, Invitrogen, Inc., (Nov. 4, 2002); ViraPower™ Lentiviral Expression System (Invitrogen, Inc., Carlsbad, Calif.) and ViraPower™ Lentiviral Expression System Instruction Manual 25-0501 version E, Invitrogen, Inc., (Dec. 8, 2003); and Complete Control® Retroviral Inducible Mammalian Expression System (Stratagene, La Jolla, Calif.) and Complete Control® Retroviral Inducible Mammalian Expression System Instruction Manual, 064005e.

The methods disclosed in the present specification include, in part, expressing a modified Clostridial toxin from a polynucleotide molecule. It is envisioned that any of a variety of expression systems may be useful for expressing a modified Clostridial toxin from a polynucleotide molecule disclosed in the present specification, including, without limitation, cell-based systems and cell-free expression systems. Cell-based systems include, without limitation, viral expression systems, prokaryotic expression systems, yeast expression systems, baculoviral expression systems, insect expression systems and mammalian expression systems. Cell-free systems include, without limitation, wheat germ extracts, rabbit reticulocyte extracts and E. coli extracts and generally are equivalent to the method disclosed herein. Expression of a polynucleotide molecule using an expression system can include any of a variety of characteristics including, without limitation, inducible expression, non-inducible expression, constitutive expression, viral-mediated expression, stably-integrated expression, and transient expression. Expression systems that include well-characterized vectors, reagents, conditions and cells are well-established and are readily available from commercial vendors that include, without limitation, Ambion, Inc. Austin, Tex.; BD Biosciences-Clontech, Palo Alto, Calif.; BD Biosciences Pharmingen, San Diego, Calif.; Invitrogen, Inc, Carlsbad, Calif.; QIAGEN, Inc., Valencia, Calif.; Roche Applied Science, Indianapolis, Ind.; and Stratagene, La Jolla, Calif. Non-limiting examples on the selection and use of appropriate heterologous expression systems are described in e.g., PROTEIN EXPRESSION. A PRACTICAL APPROACH(S. J. Higgins and B. David Hames eds., Oxford University Press, 1999); Joseph M. Fernandez & James P. Hoeffler, GENE EXPRESSION SYSTEMS. USING NATURE FOR THE ART OF EXPRESSION (Academic Press, 1999); and Meena Rai & Harish Padh, Expression Systems for Production of Heterologous Proteins, 80(9) Curr. Sci. 1121-1128, (2001). These protocols are routine procedures well within the scope of one skilled in the art and from the teaching herein.

A variety of cell-based expression procedures are useful for expressing a modified Clostridial toxin encoded by polynucleotide molecule disclosed in the present specification. Examples included, without limitation, viral expression systems, prokaryotic expression systems, yeast expression systems, baculoviral expression systems, insect expression systems and mammalian expression systems. Viral expression systems include, without limitation, the ViraPower™ Lentiviral (Invitrogen, Inc., Carlsbad, Calif.), the Adenoviral Expression Systems (Invitrogen, Inc., Carlsbad, Calif.), the AdEasy™ XL Adenoviral Vector System (Stratagene, La Jolla, Calif.) and the ViraPort® Retroviral Gene Expression System (Stratagene, La Jolla, Calif.). Non-limiting examples of prokaryotic expression systems include the Champion™ pET Expression System (EMD Biosciences-Novagen, Madison, Wis.), the TriEx™ Bacterial Expression Systems (EMD Biosciences-Novagen, Madison, Wis.), the QIAexpress® Expression System (QIAGEN, Inc.), and the Affinity® Protein Expression and Purification System (Stratagene, La Jolla, Calif.). Yeast expression systems include, without limitation, the EasySelect™ Pichia Expression Kit (Invitrogen, Inc., Carlsbad, Calif.), the YES-Echo™ Expression Vector Kits (Invitrogen, Inc., Carlsbad, Calif.) and the SpECTRA™ S. pombe Expression System (Invitrogen, Inc., Carlsbad, Calif.). Non-limiting examples of baculoviral expression systems include the BaculoDirect™ (Invitrogen, Inc., Carlsbad, Calif.), the Bac-to-Bac® (Invitrogen, Inc., Carlsbad, Calif.), and the BD BaculoGold™ (BD Biosciences-Pharmigen, San Diego, Calif.). Insect expression systems include, without limitation, the Drosophila Expression System (DES®) (Invitrogen, Inc., Carlsbad, Calif.), InsectSelect™ System (Invitrogen, Inc., Carlsbad, Calif.) and InsectDirect™ System (EMD Biosciences-Novagen, Madison, Wis.). Non-limiting examples of mammalian expression systems include the T-REx™ (Tetracycline-Regulated Expression) System (Invitrogen, Inc., Carlsbad, Calif.), the Flp-In™ T-REx™ System (Invitrogen, Inc., Carlsbad, Calif.), the pcDNA™ system (Invitrogen, Inc., Carlsbad, Calif.), the pSecTag2 system (Invitrogen, Inc., Carlsbad, Calif.), the Exchanger® System, InterPlay™ Mammalian TAP System (Stratagene, La Jolla, Calif.), Complete Control® Inducible Mammalian Expression System (Stratagene, La Jolla, Calif.) and LacSwitch® II Inducible Mammalian Expression System (Stratagene, La Jolla, Calif.).

Another procedure of expressing a modified Clostridial toxin encoded by polynucleotide molecule disclosed in the present specification employs a cell-free expression system such as, without limitation, prokaryotic extracts and eukaryotic extracts. Non-limiting examples of prokaryotic cell extracts include the RTS 100 E. coli HY Kit (Roche Applied Science, Indianapolis, Ind.), the ActivePro In Vitro Translation Kit (Ambion, Inc., Austin, Tex.), the EcoPro™ System (EMD Biosciences-Novagen, Madison, Wis.) and the Expressway™ Plus Expression System (Invitrogen, Inc., Carlsbad, Calif.). Eukaryotic cell extract include, without limitation, the RTS 100 Wheat Germ CECF Kit (Roche Applied Science, Indianapolis, Ind.), the TnT® Coupled Wheat Germ Extract Systems (Promega Corp., Madison, Wis.), the Wheat Germ IVT™ Kit (Ambion, Inc., Austin, Tex.), the Retic Lysate IVT™ Kit (Ambion, Inc., Austin, Tex.), the PROTEINscript® II System (Ambion, Inc., Austin, Tex.) and the TnT® Coupled Reticulocyte Lysate Systems (Promega Corp., Madison, Wis.).

Another aspect of the present invention provides a method of activating a modified Clostridial toxin comprising an exogenous Clostridial toxin di-chain loop region including a Clostridial toxin di-chain loop protease cleavage site from a different Clostridial toxin, such method comprising the step of incubating the modified Clostridial toxin with a Clostridial toxin di-chain loop protease under physiological conditions, wherein the Clostridial toxin di-chain loop protease is capable of cleaving the Clostridial toxin di-chain loop protease cleavage site present in the exogenous Clostridial toxin di-chain loop region and wherein cleavage of the modified Clostridial toxin by the Clostridial toxin di-chain loop protease converts the modified Clostridial toxin from its single-chain polypeptide form into its di-chain form, thereby activating the modified Clostridial toxin.

Another aspect of the present invention provides a method of activating a recombinantly-expressed Clostridial toxin, such method comprising the step of incubating the Clostridial toxin with a Clostridial toxin di-chain loop protease under physiological conditions, wherein the Clostridial toxin di-chain loop protease is capable of cleaving the Clostridial toxin di-chain loop protease cleavage site present in the Clostridial toxin di-chain loop region and wherein cleavage of the Clostridial toxin by the Clostridial toxin di-chain loop protease converts the Clostridial toxin from its single-chain polypeptide form into its di-chain form, thereby activating the recombinantly-expressed Clostridial toxin.

Aspects of the present invention provide, in part, a Clostridial toxin di-chain loop protease. As used herein, the term “Clostridial toxin di-chain loop protease” means any protease capable of selectively cleaving the P₁-P_{1′}scissile bond comprising the di-chain loop protease cleavage site. As used herein, the term “selectively” means having a highly preferred activity or effect. Thus, with reference to a Clostridial toxin di-chain loop protease, there is a discriminatory proteolytic cleavage of the P₁-P_{1′} scissile bond comprising the di-chain loop protease cleavage site. It is envisioned that any and all proteases capable of selectively cleaving the P₁-P_{1′} scissile bond comprising the di-chain loop protease cleavage site can be useful in the disclosed methods, including, without exception, a sulfhydryl proteinase. One example of a sulfhydryl proteinase is clostripain, also known as clostridiopeptidase B, endoproteinase-Arg-C, or γ-protease. See, e.g., William M. Mitchell & William F. Harrington, Purification and Properties of Clostridiopeptidase B (Clostripain), 243(18) J. Biol. Chem. 4683-4692. (1968); William M. Mitchell & William F. Harrington, Clostripain, 19 Methods Enzymol. 635-642 (1970); and Ashu A. Kembhavi, et al., Clostripain: Characterization of the Active Site, 283(2) FEBS Lett. 277-280 (1991), each of which is hereby incorporated by reference in its entirety. This two chain cysteine proteinase is highly specific for the carboxyl peptide bond of arginine. Non-limiting examples of clostripain include SEQ ID NO: 33, SEQ ID NO; 34, SEQ ID NO: 35, SEQ ID NO: 36, SEQ ID NO: 37 and SEQ ID NO: 38. Clostripain selectively hydrolysis of arginyl bonds, although lysyl bonds are cleaved at a lower rate.

A clostripain useful in aspects of the invention includes, without limitation, naturally occurring clostripain; naturally occurring clostripain variants; and non-naturally-occurring clostripain variants, such as, e.g., conservative clostripain variants, non-conservative clostripain variants and clostripain peptidomimetics. As used herein, the term “clostripain variant,” whether naturally-occurring or non-naturally-occurring, means a clostripain that has at least one amino acid change from the corresponding region of the disclosed reference sequences and can be described in percent identity to the corresponding region of that reference sequence. Any of a variety of sequence alignment methods can be used to determine percent identity, including, without limitation, global methods, local methods and hybrid methods, such as, e.g., segment approach methods. Protocols to determine percent identity are routine procedures within the scope of one skilled in the art and from the teaching herein.

As used herein, the term “naturally occurring clostripain variant” means any clostripain produced without the aid of any human manipulation, including, without limitation, clostripain isoforms produced from alternatively-spliced transcripts, clostripain isoforms produced by spontaneous mutation and clostripain subtypes. Non-limiting examples of a clostripain isoform include, e.g., BoNT/A di-chain loop region isoforms, BoNT/B di-chain loop region isoforms, BoNT/C1 di-chain loop region isoforms, BoNT/D di-chain loop region isoforms, BoNT/E di-chain loop region isoforms, BoNT/F di-chain loop region isoforms, BoNT/G di-chain loop region isoforms, TeNT di-chain loop region isoforms, BaNT di-chain loop region isoforms, and BuNT di-chain loop region isoforms. Non-limiting examples of a Clostridial toxin subtype include, e.g., BoNT/A di-chain loop region subtypes such as, e.g., a BoNT/A1 di-chain loop region, a BoNT/A2 di-chain loop region, a BoNT/A3 di-chain loop region and a BoNT/A4 di-chain loop region; BoNT/B di-chain loop region subtypes, such as, e.g., a BoNT/B1 di-chain loop region, a BoNT/B2 di-chain loop region, a BoNT/B bivalent di-chain loop region and a BoNT/B nonproteolytic di-chain loop region; BoNT/C1 di-chain loop region subtypes, such as, e.g., a BoNT/C1-1 di-chain loop region and a BoNT/C1-2 di-chain loop region; BoNT/E di-chain loop region subtypes, such as, e.g., a BoNT/E1 di-chain loop region, a BoNT/E2 di-chain loop region and a BoNT/E3 di-chain loop region; and BoNT/F di-chain loop region subtypes, such as, e.g., a BoNT/F1 di-chain loop region, a BoNT/F2 di-chain loop region, a BoNT/F3 di-chain loop region and a BoNT/F4 di-chain loop region.

As used herein, the term “non-naturally occurring clostripain variant” means any clostripain produced with the aid of human manipulation, including, without limitation, clostripain variants produced by genetic engineering using random mutagenesis or rational design and clostripain variants produced by chemical synthesis. Non-limiting examples of non-naturally occurring clostripain variants include, e.g., conservative clostripain variants, non-conservative clostripain variants and clostripain peptidomimetics.

As used herein, the term “conservative clostripain variant” means a clostripain that has at least one amino acid substituted by another amino acid or an amino acid analog that has at least one property similar to that of the original amino acid from the reference clostripain sequence. Examples of properties include, without limitation, similar size, topography, charge, hydrophobicity, hydrophilicity, lipophilicity, covalent-bonding capacity, hydrogen-bonding capacity, a physicochemical property, of the like, or any combination thereof. A conservative clostripain variant can function in substantially the same manner as the reference clostripain on which the conservative clostripain variant is based, and can be substituted for the reference clostripain in any aspect of the present invention. A conservative clostripain variant may substitute one or more amino acids, two or more amino acids, three or more amino acids, four or more amino acids or five or more amino acids from the reference clostripain on which the conservative clostripain variant is based. A conservative clostripain variant can also possess at least 50% amino acid identity, 65% amino acid identity, 75% amino acid identity, 85% amino acid identity or 95% amino acid identity to the reference clostripain on which the conservative clostripain variant is based. Non-limiting examples of a conservative clostripain variant include, e.g., conservative clostripain variants of SEQ ID NO: 33, conservative clostripain variants of SEQ ID NO: 34, conservative clostripain variants of SEQ ID NO: 35, conservative clostripain variants of SEQ ID NO: 36, conservative clostripain variants of SEQ ID NO: 37, and conservative clostripain variants of SEQ ID NO: 38.

As used herein, the term “non-conservative clostripain variant” means a clostripain in which 1) at least one amino acid is deleted from the reference clostripain on which the non-conservative clostripain variant is based; 2) at least one amino acid added to the reference clostripain on which the non-conservative clostripain is based; or 3) at least one amino acid is substituted by another amino acid or an amino acid analog that does not share any property similar to that of the original amino acid from the reference clostripain sequence. A non-conservative clostripain variant can function in substantially the same manner as the reference clostripain on which the non-conservative clostripain is based, and can be substituted for the reference clostripain in any aspect of the present invention. A non-conservative clostripain variant can add one or more amino acids, two or more amino acids, three or more amino acids, four or more amino acids, five or more amino acids, and ten or more amino acids to the reference clostripain on which the non-conservative clostripain variant is based. A non-conservative clostripain may substitute one or more amino acids, two or more amino acids, three or more amino acids, four or more amino acids or five or more amino acids from the reference clostripain on which the non-conservative clostripain variant is based. A non-conservative clostripain variant can also possess at least 50% amino acid identity, 65% amino acid identity, 75% amino acid identity, 85% amino acid identity or 95% amino acid identity to the reference clostripain on which the non-conservative clostripain variant is based. Non-limiting examples of a non-conservative clostripain variant include, e.g., non-conservative clostripain variants of SEQ ID NO: 33, non-conservative clostripain variants of SEQ ID NO: 34, non-conservative clostripain variants of SEQ ID NO: 35, non-conservative clostripain variants of SEQ ID NO: 36, non-conservative clostripain variants of SEQ ID NO: 37, and non-conservative clostripain variants of SEQ ID NO: 38.

As used herein, the term “clostripain peptidomimetic” means a clostripain that has at least one amino acid substituted by a non-natural oligomer that has at least one property similar to that of the first amino acid. Examples of properties include, without limitation, topography of a peptide primary structural element, functionality of a peptide primary structural element, topology of a peptide secondary structural element, functionality of a peptide secondary structural element, of the like, or any combination thereof. A clostripain peptidomimetic can function in substantially the same manner as the reference clostripain on which the clostripain peptidomimetic is based, and can be substituted for the reference clostripain in any aspect of the present invention. A clostripain peptidomimetic may substitute one or more amino acids, two or more amino acids, three or more amino acids, four or more amino acids or five or more amino acids from the reference clostripain on which the clostripain peptidomimetic is based. A clostripain peptidomimetic can also possess at least 50% amino acid identity, at least 65% amino acid identity, at least 75% amino acid identity, at least 85% amino acid identity or at least 95% amino acid identity to the reference clostripain on which the clostripain peptidomimetic is based. For examples of peptidomimetic methods see, e.g., Amy S. Ripka & Daniel H. Rich, Peptidomimetic design, 2(4) CURR. OPIN. CHEM. BIOL. 441-452 (1998); and M. Angels Estiarte & Daniel H. Rich, Peptidomimetics for Drug Design, 803-861 (BURGER'S MEDICINAL CHEMISTRY AND DRUG DISCOVERY Vol. 1 PRINCIPLE AND PRACTICE, Donald J. Abraham ed., Wiley-Interscience, 6^{th} ed 2003). Non-limiting examples of a clostripain peptidomimetic include, e.g., clostripain peptidomimetics of SEQ ID NO: 33, clostripain peptidomimetics of SEQ ID NO: 34, clostripain peptidomimetics of SEQ ID NO: 35, clostripain peptidomimetics of SEQ ID NO: 36, clostripain peptidomimetics of SEQ ID NO: 37, and clostripain peptidomimetics of SEQ ID NO: 38.

Thus, in an embodiment, a Clostridial toxin di-chain loop protease comprises a clostripain. In an aspect of this embodiment, a clostripain can be a naturally occurring clostripain variant, such as, e.g., a clostripain isoform or a clostripain subtype. In another aspect of this embodiment, a clostripain can be a non-naturally occurring clostripain variant, such as, e.g., a conservative clostripain variant, a non-conservative clostripain variant or an active clostripain fragment, or any combination thereof.

In another embodiment, a clostripain comprises a naturally occurring clostripain variant of SEQ ID NO: 33, such as, e.g., a clostripain isoform of SEQ ID NO: 33 or a clostripain subtype of SEQ ID NO: 33. In still another aspect of this embodiment, a clostripain comprises a non-naturally occurring clostripain variant of SEQ ID NO: 33, such as, e.g., a conservative clostripain variant of SEQ ID NO: 33, a non-conservative clostripain variant of SEQ ID NO: 33 or an active clostripain fragment of SEQ ID NO: 33, or any combination thereof. In yet another embodiment, a clostripain comprises a clostripain of SEQ ID NO: 33.

In other aspects of this embodiment, a clostripain comprises a polypeptide having, e.g., at least 70% amino acid identity with SEQ ID NO: 33, at least 75% amino acid identity with SEQ ID NO: 33, at least 80% amino acid identity with SEQ ID NO: 33, at least 85% amino acid identity with SEQ ID NO: 33, at least 90% amino acid identity with SEQ ID NO: 33 or at least 95% amino acid identity with SEQ ID NO: 33. In yet other aspects of this embodiment, a clostripain comprises a polypeptide having, e.g., at most 70% amino acid identity with SEQ ID NO: 33, at most 75% amino acid identity with SEQ ID NO: 33, at most 80% amino acid identity with SEQ ID NO: 33, at most 85% amino acid identity with SEQ ID NO: 33, at most 90% amino acid identity with SEQ ID NO: 33 or at most 95% amino acid identity with SEQ ID NO: 33.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 33. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 33. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 33.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 33. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 33. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 33. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 33.

In another embodiment, a clostripain comprises a naturally occurring clostripain variant of SEQ ID NO: 34, such as, e.g., a clostripain isoform of SEQ ID NO: 34 or a clostripain subtype of SEQ ID NO: 34. In still another aspect of this embodiment, a clostripain comprises a non-naturally occurring clostripain variant of SEQ ID NO: 34, such as, e.g., a conservative clostripain variant of SEQ ID NO: 34, a non-conservative clostripain variant of SEQ ID NO: 34 or an active clostripain fragment of SEQ ID NO: 34, or any combination thereof. In yet another embodiment, a clostripain comprises a clostripain of SEQ ID NO: 34.

In other aspects of this embodiment, a clostripain comprises a polypeptide having, e.g., at least 70% amino acid identity with SEQ ID NO: 34, at least 75% amino acid identity with SEQ ID NO: 34, at least 80% amino acid identity with SEQ ID NO: 34, at least 85% amino acid identity with SEQ ID NO: 34, at least 90% amino acid identity with SEQ ID NO: 34 or at least 95% amino acid identity with SEQ ID NO: 34. In yet other aspects of this embodiment, a clostripain comprises a polypeptide having, e.g., at most 70% amino acid identity with SEQ ID NO: 34, at most 75% amino acid identity with SEQ ID NO: 34, at most 80% amino acid identity with SEQ ID NO: 34, at most 85% amino acid identity with SEQ ID NO: 34, at most 90% amino acid identity with SEQ ID NO: 34 or at most 95% amino acid identity with SEQ ID NO: 34.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 34. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 34. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 34.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 34. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 34. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 34. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 34.

In another embodiment, a clostripain comprises a naturally occurring clostripain variant of SEQ ID NO: 35, such as, e.g., a clostripain isoform of SEQ ID NO: 35 or a clostripain subtype of SEQ ID NO: 35. In still another aspect of this embodiment, a clostripain comprises a non-naturally occurring clostripain variant of SEQ ID NO: 35, such as, e.g., a conservative clostripain variant of SEQ ID NO: 35, a non-conservative clostripain variant of SEQ ID NO: 35 or an active clostripain fragment of SEQ ID NO: 35, or any combination thereof. In yet another embodiment, a clostripain comprises a clostripain of SEQ ID NO: 35.

In other aspects of this embodiment, a clostripain comprises a polypeptide having, e.g., at least 70% amino acid identity with SEQ ID NO: 35, at least 75% amino acid identity with SEQ ID NO: 35, at least 80% amino acid identity with SEQ ID NO: 35, at least 85% amino acid identity with SEQ ID NO: 35, at least 90% amino acid identity with SEQ ID NO: 35 or at least 95% amino acid identity with SEQ ID NO: 35. In yet other aspects of this embodiment, a clostripain comprises a polypeptide having, e.g., at most 70% amino acid identity with SEQ ID NO: 35, at most 75% amino acid identity with SEQ ID NO: 35, at most 80% amino acid identity with SEQ ID NO: 35, at most 85% amino acid identity with SEQ ID NO: 35, at most 90% amino acid identity with SEQ ID NO: 35 or at most 95% amino acid identity with SEQ ID NO: 35.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 35. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 35. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 35.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 35. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 35. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 35. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 35.

In another embodiment, a clostripain comprises a naturally occurring clostripain variant of SEQ ID NO: 36, such as, e.g., a clostripain isoform of SEQ ID NO: 36 or a clostripain subtype of SEQ ID NO: 36. In still another aspect of this embodiment, a clostripain comprises a non-naturally occurring clostripain variant of SEQ ID NO: 36, such as, e.g., a conservative clostripain variant of SEQ ID NO: 36, a non-conservative clostripain variant of SEQ ID NO: 36 or an active clostripain fragment of SEQ ID NO: 36, or any combination thereof. In yet another embodiment, a clostripain comprises a clostripain of SEQ ID NO: 36.

In other aspects of this embodiment, a clostripain comprises a polypeptide having, e.g., at least 70% amino acid identity with SEQ ID NO: 36, at least 75% amino acid identity with SEQ ID NO: 36, at least 80% amino acid identity with SEQ ID NO: 36, at least 85% amino acid identity with SEQ ID NO: 36, at least 90% amino acid identity with SEQ ID NO: 36 or at least 95% amino acid identity with SEQ ID NO: 36. In yet other aspects of this embodiment, a clostripain comprises a polypeptide having, e.g., at most 70% amino acid identity with SEQ ID NO: 36, at most 75% amino acid identity with SEQ ID NO: 36, at most 80% amino acid identity with SEQ ID NO: 36, at most 85% amino acid identity with SEQ ID NO: 36, at most 90% amino acid identity with SEQ ID NO: 36 or at most 95% amino acid identity with SEQ ID NO: 36.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 36. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 36. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 36.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 36. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 36. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 36. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 36.

In another embodiment, a clostripain comprises a naturally occurring clostripain variant of SEQ ID NO: 37, such as, e.g., a clostripain isoform of SEQ ID NO: 37 or a clostripain subtype of SEQ ID NO: 37. In still another aspect of this embodiment, a clostripain comprises a non-naturally occurring clostripain variant of SEQ ID NO: 37, such as, e.g., a conservative clostripain variant of SEQ ID NO: 37, a non-conservative clostripain variant of SEQ ID NO: 37 or an active clostripain fragment of SEQ ID NO: 37, or any combination thereof. In yet another embodiment, a clostripain comprises a clostripain of SEQ ID NO: 37.

In other aspects of this embodiment, a clostripain comprises a polypeptide having, e.g., at least 70% amino acid identity with SEQ ID NO: 37, at least 75% amino acid identity with SEQ ID NO: 37, at least 80% amino acid identity with SEQ ID NO: 37, at least 85% amino acid identity with SEQ ID NO: 37, at least 90% amino acid identity with SEQ ID NO: 37 or at least 95% amino acid identity with SEQ ID NO: 37. In yet other aspects of this embodiment, a clostripain comprises a polypeptide having, e.g., at most 70% amino acid identity with SEQ ID NO: 37, at most 75% amino acid identity with SEQ ID NO: 37, at most 80% amino acid identity with SEQ ID NO: 37, at most 85% amino acid identity with SEQ ID NO: 37, at most 90% amino acid identity with SEQ ID NO: 37 or at most 95% amino acid identity with SEQ ID NO: 37.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 37. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 37. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 37.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 37. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 37. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 37. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 37.

In another embodiment, a clostripain comprises a naturally occurring clostripain variant of SEQ ID NO: 38, such as, e.g., a clostripain isoform of SEQ ID NO: 38 or a clostripain subtype of SEQ ID NO: 38. In still another aspect of this embodiment, a clostripain comprises a non-naturally occurring clostripain variant of SEQ ID NO: 38, such as, e.g., a conservative clostripain variant of SEQ ID NO: 38, a non-conservative clostripain variant of SEQ ID NO: 38 or an active clostripain fragment of SEQ ID NO: 38, or any combination thereof. In yet another embodiment, a clostripain comprises a clostripain of SEQ ID NO: 38.

In other aspects of this embodiment, a clostripain comprises a polypeptide having, e.g., at least 70% amino acid identity with SEQ ID NO: 38, at least 75% amino acid identity with SEQ ID NO: 38, at least 80% amino acid identity with SEQ ID NO: 38, at least 85% amino acid identity with SEQ ID NO: 38, at least 90% amino acid identity with SEQ ID NO: 38 or at least 95% amino acid identity with SEQ ID NO: 38. In yet other aspects of this embodiment, a clostripain comprises a polypeptide having, e.g., at most 70% amino acid identity with SEQ ID NO: 38, at most 75% amino acid identity with SEQ ID NO: 38, at most 80% amino acid identity with SEQ ID NO: 38, at most 85% amino acid identity with SEQ ID NO: 38, at most 90% amino acid identity with SEQ ID NO: 38 or at most 95% amino acid identity with SEQ ID NO: 38.

In other aspects of this embodiment, a clostripain comprises a polypeptide having, e.g., at most one, two, three, four, five, six, seven, eight, nine, 10, 20, 30, 40, 50, or 100 non-contiguous amino acid substitutions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 non-contiguous amino acid substitutions relative to SEQ ID NO: 38. In yet other aspects of this embodiment, a clostripain comprises a polypeptide having, e.g., at most one, two, three, four, five, six, seven, eight, nine, 10, 20, 30, 40, 50, or 100 non-contiguous amino acid deletions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 non-contiguous amino acid deletions relative to SEQ ID NO: 38. In still other aspects of this embodiment, a clostripain comprises a polypeptide having, e.g., at most one, two, three, four, five, six, seven, eight, nine, 10, 20, 30, 40, 50, or 100 non-contiguous amino acid additions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 non-contiguous amino acid additions relative to SEQ ID NO: 38.

In other aspects of this embodiment, a clostripain comprises a polypeptide having, e.g., at most one, two, three, four, five, six, seven, eight, nine, 10, 20, 30, 40, 50, or 100 contiguous amino acid substitutions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 contiguous amino acid substitutions relative to SEQ ID NO: 38. In yet other aspects of this embodiment, a clostripain comprises a polypeptide having, e.g., at most one, two, three, four, five, six, seven, eight, nine, 10, 20, 30, 40, 50, or 100 contiguous amino acid deletions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 contiguous amino acid deletions relative to SEQ ID NO: 38. In still other aspects of this embodiment, a clostripain comprises a polypeptide having, e.g., at most one, two, three, four, five, six, seven, eight, nine, 10, 20, 30, 40, 50, or 100 contiguous amino acid additions relative to SEQ ID NO: 38. In other aspects of this embodiment, a clostripain comprises a polypeptide having, e.g., at least one, two, three, four, five, six, seven, eight, nine, 10, 20, 30, 40, 50, or 100 contiguous amino acid additions relative to SEQ ID NO: 38.

Other examples of a di-chain loop protease include SEQ ID NO: 39, SEQ ID NO: 40, SEQ ID NO: 41, SEQ ID NO: 42, SEQ ID NO: 43, SEQ ID NO: 44, SEQ ID NO: 45, SEQ ID NO: 46, SEQ ID NO: 47, SEQ ID NO: 48, and SEQ ID NO: 49, or a naturally-occurring of non-naturally occurring variant.

It is envisioned that any and all assay conditions suitable for proteolytic cleavage of the scissile bond comprising a di-chain protease cleavage site by a di-chain loop protease are useful in the methods disclosed in the present specification, such as, e.g., linear assay conditions and non-linear assay conditions. In an embodiment of the present invention, the assay conditions are linear. In an aspect of this embodiment, the assay amount of a recombinantly-expressed or a modified Clostridial toxin is in excess. In an aspect of this embodiment, the assay amount of a di-chain loop protease is in excess. In another aspect of this embodiment, the assay amount of a recombinantly-expressed or a modified Clostridial toxin is rate-limiting. In another aspect of this embodiment, the assay amount of a di-chain loop protease is rate-limiting.

In other aspects of this embodiment, assay conditions suitable for activating a recombinantly-expressed or modified Clostridial toxin can be provided such that, e.g., at least 10% of the recombinantly-expressed or modified Clostridial toxin is cleaved, at least 20% of the recombinantly-expressed or modified Clostridial toxin is cleaved, at least 30% of the recombinantly-expressed or modified Clostridial toxin is cleaved, at least 40% of the recombinantly-expressed or modified Clostridial toxin is cleaved, at least 50% of the recombinantly-expressed or modified Clostridial toxin is cleaved, at least 60% of the recombinantly-expressed or modified Clostridial toxin is cleaved, at least 70% of the recombinantly-expressed or modified Clostridial toxin is cleaved, at least 80% of the recombinantly-expressed or modified Clostridial toxin is cleaved or at least 90% of the recombinantly-expressed or modified Clostridial toxin is cleaved. In other aspects of this embodiment, conditions suitable for activating a recombinantly-expressed or a modified Clostridial toxin can be provided such that, e.g., at most 10% of the recombinantly-expressed or modified Clostridial toxin is cleaved, at most 20% of the recombinantly-expressed or modified Clostridial toxin is cleaved, at most 30% of the recombinantly-expressed or modified Clostridial toxin is cleaved, at most 40% of the recombinantly-expressed or modified Clostridial toxin is cleaved, at most 50% of the recombinantly-expressed or modified Clostridial toxin is cleaved, at most 60% of the recombinantly-expressed or modified Clostridial toxin is cleaved, at most 70% of the recombinantly-expressed or modified Clostridial toxin is cleaved, at most 80% of the recombinantly-expressed or modified Clostridial toxin is cleaved or at most 90% of the recombinantly-expressed or modified Clostridial toxin is cleaved. In another aspect of this embodiment, conditions suitable for activating a recombinantly-expressed or a modified Clostridial toxin can be provided such that 100% of the recombinantly-expressed or modified Clostridial toxin is cleaved. In another aspect of this embodiment, the conditions suitable for activating a recombinantly-expressed or a modified Clostridial toxin are provided such that the assay is linear. In another aspect of this embodiment, the conditions suitable for activating a recombinantly-expressed or a modified Clostridial toxin are provided such that the assay is non-linear.

The presence of calcium ions is essential for Clostripain proteolytic activity. Thus, in another embodiment, assay conditions suitable for activating a recombinantly-expressed or modified Clostridial toxin include a source of calcium, such as calcium chloride or calcium acetate. In aspects of this embodiment, assay conditions include calcium in the range of about 0.1 μM to about 500 μM, for example, about 0.1 μM to about 50 μM, about 0.1 μM to about 5 μM, about 1 μM to about 500 μM, about 1 μM to about 50 μM, about 1 μM to about 5 μM, about 5 μM to about 15 μM, and about 5 μM to about 10 μM. One skilled in the art understands that calcium chelators such as EGTA generally are excluded from an assay condition used to activate a recombinantly-expressed or modified Clostridial toxin. Potent inhibitors of clostripain activity include, e.g., oxidizing agents, thiol-blocking agents, Co²⁺, Cu²⁺, Cd²⁺ and heavy metal ions. Citrate, borate and Tris partially inhibit Clostripain proteolytic activity.

In addition, the activity of clostripain depends upon a cysteine thiol group, so a reducing agent such as, e.g., dithiothreitol (DTT), cysteine, β-mercaptoethanol, dimethylsulfoxide (DMSO), or other sulfhydryl containing reagents is included in the assay buffer. In aspect of this embodiment, concentrations for a reducing agent may include, e.g., at least 10 nM, at least 50 nM, at least 100 nM, at least 500 nM, at least 1 mM, at least 10 mM or at least 100 mM. In another aspect of this embodiment, concentrations for a reducing agent may include, e.g., at most 10 nM, at most 50 nM, at most 100 nM, at most 500 nM, at most 1 mM, at most 10 mM or at most 100 mM. Non-limiting examples of how to make and use specific reducing agents are described in, e.g., MOLECULAR CLONING, A LABORATORY MANUAL, supra, (2001); and CURRENT PROTOCOLS IN MOLECULAR BIOLOGY, supra, (2004).

In another embodiment, the amount of di-chain loop protease used to activate a recombinantly-expressed or a modified Clostridial toxin can be in the range of about 0.001 μg to about 500 μg, for example, from about 0.001 μg to about 05 μg, about 0.001 μg to about 5 μg, about 0.001 μg to about 50 pg, about 0.01 μg to about 05 μg, 0.01 μg to about 5 μg, about 0.01 μg to about 50 μg, about 0.01 μg to about 500 μg, about 0.1 μg to about 05 μg, 0.1 μg to about 5 μg, about 0.1 μg to about 50 μg, about 0.1 pg to about 500 μg, about 1 μg to about 05 μg, about 1 μg to about 5 μg, about 1 μg to about 50 μg, or about 1 μg to about 500 μg.

In another embodiment, the pH of the buffer used in the method to activate a recombinantly-expressed or a modified Clostridial toxin can be in the range of about pH 6.0 to about pH 9.5, for example, from about pH 6.0 to about pH 9.0, about pH 6.0 to about pH 8.5, about pH 6.0 to about pH 8.0, about pH 6.0 to about pH 7.5, about pH 7.0 to about pH 9.0, about pH 7.0 to about pH 8.5, about pH 7.0 to about pH 8.0, about pH 7.2 to about pH 8.0, about pH 7.2 to about pH 7.8, about pH 7.2 to about pH 7.6, about pH 7.2 to about pH 7.4, about pH 7.4 to about pH 8.0, about pH 7.4 to about pH 7.8, about pH 7.4 to about pH 7.6, about pH 7.4 to about pH 8.0, about pH 7.4 to about pH 7.8, or about pH 7.4 to about pH 7.6.

In a further embodiment, it is also envisioned that any and all buffers that allow the cleavage of the di-chain loop protease cleavage site by a di-chain loop protease can optionally be used in the activation methods disclosed in the present specification. Assay buffers can be varied as appropriate by one skilled in the art and generally depend, in part, on the pH value desired for the assay and the detection means employed. Therefore, aspects of this embodiment may optionally include, e.g., 2-amino-2-hydroxymethyl-1,3-propanediol (Tris) buffers; Phosphate buffers, such as, e.g., potassium phosphate buffers and sodium phosphate buffers; Good buffers, such as, e.g., 2-(N-morpholino) ethanesulfonic acid (MES), piperazine-N,N′-bis(2-ethanesulfonic acid) (PIPES), N,N′-bis(2-hydroxyethyl)-2-aminoethanesulfonic acid (BES),3-(N-morpholino) propanesulfonic acid (MOPS), N-(2-hydroxyethyl)piperazine-N′-(2-ethanesulfonic acid) (HEPES), piperazine-N,N′-bis(2-hydroxypropanesulfonic acid) (POPSO), N-tris(hydroxymethyl)methylglycine (Tricine), N-tris(hydroxymethyl)methyl-3-aminopropanesulfonic acid (TAPS), 3-[(1,1-dimethyl-2-hydroxyethyl)amino]-2-hydroxypropanesulfonic acid (AMPSO), 3-(cyclohexylamino)-2-hydroxy-1-propanesulfonic acid (CAPSO), and 3-(cyclohexylamino)-1-propanesulfonic acid (CAPS); saline buffers, such as, e.g., Phosphate-buffered saline (PBS), HEPES-buffered saline, and Tris-buffered saline (TBS); Acetate buffers, such as, e.g., magnesium acetate, potassium actetate, and Tris acetate; and the like, or any combination thereof. In addition, the buffer concentration in a method disclosed in the present specification can be varied as appropriate by one skilled in the art and generally depend, in part, on the buffering capacity of a particular buffer being used and the detection means employed. Thus, aspects of this embodiment may include a buffer concentration of, e.g., at least 1 mM, at least 5 mM, at least 10 mM, at least 20 mM, at least 30 mM, at least 40 mM, at least 50 mM, at least 60 mM, at least 70 mM, at least 80 mM, at least 90 mM, or at least 100 mM. Non-limiting examples of how to make and use specific buffers are described in, e.g., MOLECULAR CLONING, A LABORATORY MANUAL, supra, (2001); and CURRENT PROTOCOLS IN MOLECULAR BIOLOGY, supra, (2004).

In a further embodiment, it is also envisioned that any and all salts that allow the cleavage of the di-chain loop protease cleavage site by a di-chain loop protease can optionally be used in the activation methods disclosed in the present specification. Assay salts can be varied as appropriate by one skilled in the art and generally depend, in part, on the physiological conditions desired for the assay and the detection means employed. Therefore, aspects of this embodiment may optionally include, e.g., sodium chloride, potassium chloride, calcium chloride, magnesium chloride, manganese chloride, zinc chloride, magnesium sulfate, zinc sulfate, and the like, or any combination thereof. In addition, the salt concentration in a method disclosed in the present specification can be varied as appropriate by one skilled in the art and generally depend, in part, on the buffering capacity of a particular buffer being used and the detection means employed. Thus, aspects of this embodiment may include a salt concentration of, e.g., at least 1 mM, at least 5 mM, at least 10 mM, at least 20 mM, at least 30 mM, at least 40 mM, at least 50 mM, at least 60 mM, at least 70 mM, at least 80 mM, at least 90 mM, or at least 100 mM. Non-limiting examples of how to make and use specific salts are described in, e.g., MOLECULAR CLONING, A LABORATORY MANUAL, supra, (2001); and CURRENT PROTOCOLS IN MOLECULAR BIOLOGY, supra, (2004).

In another embodiment, the concentration of a recombinantly-expressed or a modified Clostridial toxin to be activated can be in the range of about 0.0001 ng/ml to 500 μg/ml toxin, for example, about 0.0001 ng/ml to 50 μg/ml toxin, 0.001 ng/ml to 500 μg/ml toxin, 0.001 ng/ml to 50 μg/ml toxin, 0.0001 to 5000 ng/ml toxin, 0.001 ng/ml to 5000 ng/ml, 0.01 ng/ml to 5000 ng/ml, 0.1 ng/ml to 5000 ng/ml, 0.1 ng/ml to 500 ng/ml, 0.1 ng/ml to 50 ng/ml, 1 ng/ml to 5000 ng/ml, 1 ng/ml to 500 ng/ml, 1 ng/ml to 50 ng/ml, 10 ng/ml to 5000 ng/ml, 10 ng/ml to 500 ng/ml, 50 ng/ml to 5000 ng/ml, 50 ng/ml to 500 ng/ml or 100 ng/ml to 5000 ng/ml toxin. In another embodiment, the concentration of a recombinantly-expressed or a modified Clostridial toxin to be activated can be in the range of about 0.1 pM to 500 μM, 0.1 pM to 100 μM, 0.1 pM to 10 μM, 0.1 pM to 1 μM, 0.1 pM to 500 nM, 0.1 pM to 100 nM, 0.1 pM to 10 nM, 0.1 pM to 1 nM, 0.1 pM to 500 pM, 0.1 pM to 100 pM, 0.1 pM to 50 pM, 0.1 pM to 10 pM, 1 pM to 500 μM, 1 pM to 100 μM, 1 pM to 10 μM, 1 pM to 1 μM, 1 pM to 500 nM, 1 pM to 100 nM, 1 pM to 10 nM, 1 pM to 1 nM, 1 pM to 500 pM, 1 pM to 100 pM, 1 pM to 50 pM, 1 pM to 10 pM, 10 pM to 500 μM, 10 pM to 100 μM, 10 pM to 10 μM, 10 pM to 10 μM, 10 pM to 500 nM, 10 pM to 100 nM, 10 pM to 10 nM, 10 pM to 1 nM, 10 pM to 500 pM, 10 pM to 100 pM, 10 pM to 50 pM, 100 pM to 500 μM, 100 pM to 100 μM, 100 pM to 10 μM, 100 pM to 1 μM, 100 pM to 500 nM, 100 pM to 100 nM, 100 pM to 10 nM, 100 pM to 1 nM, 100 pM to 500 pM 1 nM to 500 μM, 1 nM to 100 μM, 1 nM to 10 μM, 1 nM to 1 μM, 1 nM to 500 nM, 1 nM to 100 nM, 1 nM to 50 nM, 1 nM to 10 nM, 3 nM to 100 nM toxin. One skilled in the art understands that the concentration of a recombinantly-expressed or a modified Clostridial toxin to be activated will depend on the particular recombinantly-expressed or modified Clostridial toxin to be activated, as well as, the particular di-chain loop protease used, the presence of inhibitory components, and the assay conditions.

In still another embodiment, it is envisioned that any and all temperatures that allow activation of a recombinantly-expressed or a modified Clostridial toxin by a di-chain loop protease can be used in methods disclosed in the present specification. Assay temperatures can be varied as appropriate by one skilled in the art and generally depend, in part, on the concentration, purity of the recombinantly-expressed or modified Clostridial toxin, the activity of the di-chain loop protease, the assay time or the convenience of the artisan. Thus, an assay temperature should not be as low as to cause the solution to freeze and should not be as high as to denature a recombinantly-expressed or a modified Clostridial toxin or a di-chain loop protease disclosed in the present specification. In an aspect of this embodiment, the activation method is performed within a temperature range above 0° C., but below 40° C. In another aspect of this embodiment, the activation method is performed within a temperature range of about 4° C. to about 37° C. In yet another aspect of this embodiment, the activation method is performed within a temperature range of about 2° C. to 10° C. In yet another aspect of this embodiment, the activation method is performed at about 4° C. In still another aspect of this embodiment, the activation method is performed within a temperature range of about 10° C. to about 18° C. In still another aspect of this embodiment, the activation method is performed at about 16° C. In yet another aspect of this embodiment, the activation method is performed within a temperature range of about 18° C. to about 32° C. In yet another aspect of this embodiment, the activation method is performed at about 20° C. In another aspect of this embodiment, the activation method is performed within a temperature range of about 32° C. to about 40° C. In another aspect of this embodiment, the activation method is performed at about 37° C.

In still another embodiment, it is envisioned that any and all times sufficient for activating a recombinantly-expressed or a modified Clostridial toxin can be used in methods disclosed in the present specification. Assay times can be varied as appropriate by the skilled artisan and generally depend, in part, on the concentration and purity of the recombinantly-expressed or a modified Clostridial toxin, activity of the di-chain loop protease, incubation temperature or the convenience of the artisan. Assay times generally vary, without limitation, in the range of about 15 minutes to about 4 hours, 30 minutes to 8 hours, 1 hour to 12 hours, 2 hours to 24 hours, 4 hours to 48 hours, 6 hours to 72 hours. It is understood that assays can be terminated at any time.

Aspects of the present invention can also be described as follows:
1. A modified Clostridial toxin comprising an exogenous Clostridial toxin di-chain loop region including a di-chain protease cleavage site; wherein the Clostridial toxin di-chain loop region replaces an endogenous Clostridial toxin di-chain loop region.
2. The modified Clostridial toxin of 1, wherein the Clostridial toxin being modified is BoNT/B, BoNT/C1, BoNT/D, BoNT/E, BoNT/F, BoNT/G, TeNT, BaNT, or BuNT and the exogenous Clostridial toxin di-chain loop region is BoNT/A.
3. The modified Clostridial toxin of 2, wherein the modified Clostridial toxin is SEQ ID NO: 50, SEQ ID NO: 51, SEQ ID NO: 52, SEQ ID NO: 53, SEQ ID NO: 54, SEQ ID NO: 55, SEQ ID NO: 56, SEQ ID NO: 57, or SEQ ID NO: 58.
4. The modified Clostridial toxin of 1, wherein the Clostridial toxin being modified is BoNT/A, BoNT/C1, BoNT/D, BoNT/E, BoNT/F, BoNT/G, TeNT, BaNT, or BuNT and the exogenous Clostridial toxin di-chain loop region is BoNT/B.
5. The modified Clostridial toxin of 1, wherein the Clostridial toxin being modified is BoNT/A, BoNT/B, BoNT/C1, BoNT/D, BoNT/E, BoNT/G, TeNT, BaNT, or BuNT and the exogenous Clostridial toxin di-chain loop region is BoNT/F.
6. The modified Clostridial toxin of 1, wherein the Clostridial toxin being modified is BoNT/A, BoNT/B, BoNT/C1, BoNT/D, BoNT/E, BoNT/G, BaNT, or BuNT and the exogenous Clostridial toxin di-chain loop region is TeNT.
7. The modified Clostridial toxin of 1, wherein the Clostridial toxin being modified is BoNT/A, BoNT/B, BoNT/C1, BoNT/D, BoNT/E, BoNT/G, TeNT, or BuNT and the exogenous Clostridial toxin di-chain loop region is BaNT.
8. A polynucleotide molecule encoding a modified Clostridial toxin according to any one of 1-7.
9. The polynucleotide molecule of 8, further comprising an expression vector.
10. A method of producing a modified Clostridial toxin comprising the step of expressing in a cell a polynucleotide molecule according to 9, wherein expression from the polynucleotide molecule produces the encoded modified Clostridial toxin.
11. A method of producing a modified Clostridial toxin comprising the steps of:
   a. introducing into a cell a polynucleotide molecule as defined in 9; and
   b. expressing the polynucleotide molecule, wherein expression from the polynucleotide molecule produces the encoded modified Clostridial toxin.
12. A method of activating a modified Clostridial toxin, the method comprising the step of incubating a modified Clostridial toxin according to any one of 1-7 with a di-chain loop protease, wherein cleavage of the modified Clostridial toxin by the di-chain loop protease converts the modified Clostridial toxin from its single-chain polypeptide form into its di-chain form, thereby activating the modified Clostridial toxin.
13. A method of activating a modified Clostridial toxin, the method comprising the step of incubating a modified Clostridial toxin according to any one of 2 with a BoNT/A di-chain loop protease under physiological conditions;
   wherein cleavage of the modified Clostridial toxin by the BoNT/A di-chain loop protease converts the modified Clostridial toxin from its single-chain polypeptide form into its di-chain form, thereby activating the modified Clostridial toxin.
14. A method of either 13 or 14, wherein the BoNT/A toxin di-chain loop protease is SEQ ID NO: 33, SEQ ID NO: 34, SEQ ID NO: 35, SEQ ID NO: 36, SEQ ID NO: 37, or SEQ ID NO: 38; and
15. A method of activating a recombinatly-expressed Clostridial toxin, the method comprising the steps of:
   a. expressing in an aerobic bacterial cell a polynucleotide molecule encoding a Clostridial toxin;
   b. purifying the Clostridial toxin; and
   c. incubating the purified Clostridial toxin with a Clostridial toxin di-chain loop protease under physiological conditions;
   wherein cleavage of the purified Clostridial toxin by the Clostridial toxin di-chain loop protease converts the purified Clostridial toxin from its single-chain polypeptide form into its di-chain form, thereby activating the recombinantly-expressed Clostridial toxin.
16. A method of activating a recombinantly-expressed BoNT/A, the method comprising the steps of:
   a. expressing in an aerobic bacterial cell a polynucleotide molecule encoding a BoNT/A;
   b. purifying the BoNT/A; and
   c. incubating the purified BoNT/A with a BoNT/A di-chain loop protease under physiological conditions;
   wherein the BoNT/A toxin di-chain loop protease is SEQ ID NO: 33, SEQ ID NO: 34, SEQ ID NO: 35, SEQ ID NO: 36, SEQ ID NO: 37, or SEQ ID NO: 38; and
   wherein cleavage of the purified BoNT/A by the BoNT/A di-chain loop protease converts the purified BoNT/A from its single-chain polypeptide form into its di-chain form, thereby activating the recombinantly-expressed BoNT/A.
17. A modified Clostridial toxin comprising:
   a) a Clostridial toxin enzymatic domain;
   b) a Clostridial toxin translocation domain;
   c) a targeting moiety;
   d) an exogenous Clostridial toxin di-chain loop region including a di-chain protease cleavage site; wherein the Clostridial toxin di-chain loop region replaces an endogenous Clostridial toxin di-chain loop region.
18. The modified Clostridial toxin of 17, wherein the targeting moiety is one disclosed in Steward, supra, International Patent Publication No. 2006/008956; Steward, supra, U.S. patent application Ser. No. 11/776,043; Steward, supra, International Patent Publication No. 2006/009831; Steward, supra, U.S. Patent Publication No. 2006/0211619; Steward, supra, U.S. patent application Ser. No. 11/776,052; Foster, supra, U.S. Pat. No. 5,989,545; Shone, supra, U.S. Pat. No. 6,461,617; Quinn, supra, U.S. Pat. No. 6,632,440; Steward, supra, U.S. Pat. No. 6,843,998; Donovan, supra, U.S. Pat. No. 7,138,127; Foster, supra, U.S. Patent Publication 2003/0180289; Dolly, supra, U.S. Pat. No. 7,132,259; Foster, supra, International Patent Publication WO 2005/023309; Steward, supra, U.S. patent application Ser. No. 11/376,696; Foster, supra, International Patent Publication WO 2006/059093; Foster, supra, International Patent Publication WO 2006/059105; or Steward, supra, U.S. patent application Ser. No. 11/776,075.

### EXAMPLES

The following non-limiting examples are provided for illustrative purposes only in order to facilitate a more complete understanding of disclosed embodiments and are in no way intended to limit any of the embodiments disclosed in the present specification.

### Example 1

### Construction of Modified Clostridial Toxins Comprising a Di-Chain Loop Protease Cleavage Site from a Different Clostridial Toxin

This example illustrates how to make a modified Clostridial toxin comprising a di-chain loop protease cleavage site from a different Clostridial toxin located in the di-chain loop region of the modified toxin.

A polynucleotide molecule based on BoNT/E-DiA (SEQ ID NO: 50) will be synthesized using standard procedures (BlueHeron® Biotechnology, Bothell, Wash.). BoNT/E-DiA is a toxin which is modified to replace the endogenous di-chain loop region of BoNT/E (SEQ ID NO: 15) with the BoNT/A di-chain loop region of SEQ ID NO: 11. Oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the SmaI site to generate pUCBHB1/BoNT/E-DiA. The synthesized polynucleotide molecule is verified by sequencing using Big Dye Terminator™ Chemistry 3.1 (Applied Biosystems, Foster City, Calif.) and an ABI 3100 sequencer (Applied Biosystems, Foster City, Calif.).

If desired, an expression optimized polynucleotide molecule based on BoNT/E-DiA (SEQ ID NO: 50) can be synthesized in order to improve expression in an Escherichia coli strain. The polynucleotide molecule encoding the BoNT/E-DiA can be modified to 1) contain synonymous codons typically present in native polynucleotide molecules of an Escherichia coli strain; 2) contain a G+C content that more closely matches the average G+C content of native polynucleotide molecules found in an Escherichia coli strain; 3) reduce polymononucleotide regions found within the polynucleotide molecule; and/or 4) eliminate internal regulatory or structural sites found within the polynucleotide molecule, see, e.g., Lance E. Steward et al. Optimizing Expression of Active Botulinum Toxin Type E, International Patent Publication No. WO 2006/011966 (Feb. 2, 2006); Lance E. Steward et al. Optimizing Expression of Active Botulinum Toxin Type A, International Patent Publication No. WO 2006/017749 (Feb. 16, 2006), each of which is hereby incorporated by reference in its entirety. Once sequence optimization is complete, oligonucleotides of 20 to 50 bases in length are synthesized using standard phosphoramidite synthesis. These oligonucleotides are hybridized into double stranded duplexes that are ligated together to assemble the full-length polynucleotide molecule. This polynucleotide molecule is cloned using standard molecular biology methods into a pUCBHB1 vector at the SmaI site to generate pUCBHB1/BoNT/E-DiA. The synthesized polynucleotide molecule is verified by sequencing using Big Dye Terminator™ Chemistry 3.1 (Applied Biosystems, Foster City, Calif.) and an ABI 3100 sequencer (Applied Biosystems, Foster City, Calif.). If so desired, optimization to a different organism, such as, e.g., a yeast strain, an insect cell-line or a mammalian cell line, can be done, see, e.g., Steward, supra, International Patent Publication No. WO 2006/011966 (Feb. 2, 2006); and Steward, supra, International Patent Publication No. WO 2006/017749 (Feb. 16, 2006).

A similar cloning strategy is used to make pUCBHB1 cloning constructs for BoNT/E-DiB, a modified BoNT/E where SEQ ID NO: 15 is replaced with SEQ ID NO: 12, a BoNT/B di-chain loop region; BoNT/E-DiF, a modified BoNT/E where SEQ ID NO: 15 is replaced with SEQ ID NO: 16, a BoNT/F di-chain loop region; BoNT/E-DiBa, a modified BoNT/E where SEQ ID NO: 15 is replaced with SEQ ID NO: 19, a BaNT di-chain loop region; and BoNT/E-DiT, a modified BoNT/E where SEQ ID NO: 15 is replaced with SEQ ID NO: 18, a TeNT di-chain loop region. In addition, using a similar strategy one skilled in the art can, e.g., modify BoNT/B by replacing the BoNT/B di-chain loop region of SEQ ID NO: 12 with SEQ ID NO: 11, a BoNT/A di-chain loop region, to construct BoNT/B-Di-A (SEQ ID NO: 51); modify BoNT/C1 by replacing the BoNT/C1 di-chain loop region of SEQ ID NO: 13 with SEQ ID NO: 11, a BoNT/A di-chain loop region, to construct BoNT/C1-Di-A (SEQ ID NO: 52); modify BoNT/D by replacing the BoNT/D di-chain loop region of SEQ ID NO: 14 with SEQ ID NO: 11, a BoNT/A di-chain loop region, to construct BoNT/D-Di-A (SEQ ID NO: 53); modify BoNT/F by replacing the BoNT/F di-chain loop region of SEQ ID NO: 16 with SEQ ID NO: 11, a BoNT/A di-chain loop region, to construct BoNT/F-Di-A (SEQ ID NO: 54); modify BoNT/G by replacing the BoNT/G di-chain loop region of SEQ ID NO: 17 with SEQ ID NO: 11, a BoNT/A di-chain loop region, to construct BoNT/G-Di-A (SEQ ID NO: 55); modify TeNT by replacing the TeNT di-chain loop region of SEQ ID NO: 18 with SEQ ID NO: 11, a BoNT/A di-chain loop region, to construct TeNT-Di-A (SEQ ID NO: 56); modify BaNT by replacing the BaNT di-chain loop region of SEQ ID NO: 19 with SEQ ID NO: 11, a BoNT/A di-chain loop region, to construct BaNT-Di-A (SEQ ID NO: 57); and modify BuNT by replacing the BuNT di-chain loop region of SEQ ID NO: 20 with SEQ ID NO: 11, a BoNT/A di-chain loop region, to construct BuNT-Di-A (SEQ ID NO: 58).

To construct pET29/BoNT/E-DiA, a pUCBHB1/BoNT/E-DiA construct is digested with restriction endonucleases that 1) will excise the polynucleotide molecule encoding BoNT/E-DiA; and 2) will enable this polynucleotide molecule to be operably-linked to a pET29 vector (EMD Biosciences-Novagen, Madison, Wis.). This insert will be subcloned using a T4 DNA ligase procedure into a pET29 vector that is digested with appropriate restriction endonucleases to yield pET29/BoNT/E-DiA. The ligation mixture will be transformed into chemically competent E. coli DH5α cells (Invitrogen, Inc, Carlsbad, Calif.) using a heat shock method, will be plated on 1.5% Luria-Bertani agar plates (pH 7.0) containing 50 μg/mL of Kanamycin, and will be placed in a 37° C. incubator for overnight growth. Bacteria containing expression constructs will be identified as Kanamycin resistant colonies. Candidate constructs will be isolated using an alkaline lysis plasmid mini-preparation procedure and will be analyzed by restriction endonuclease digest mapping to determine the presence and orientation of the insert. This cloning strategy will yield a pET29 expression construct comprising the polynucleotide molecule encoding the BoNT/E-DiA operably-linked to a carboxyl terminal polyhistidine affinity binding peptide.

A similar cloning strategy can be used to make pET29 expression constructs comprising the polynucleotide molecule encoding BoNT/E-DiB, BoNT/E-DiF, BoNT/E-Ba and BoNT/E-DiT. Likewise, a similar cloning strategy will be used to make pET29 expression constructs comprising a polynucleotide molecule encoding BoNT/B-DiA, BoNT/C1-DiA, BoNT/D-DiA, BoNT/F-DiA, BoNT/G-DiA, TeNT-DiA, BaNT-DiA, and BuNT-DiA.

### Example 2

### Expression of Modified Clostridial Toxins in a Bacterial Cell

The following example illustrates a procedure useful for expressing any of the modified Clostridial toxins disclosed in the present specification in a bacterial cell.

An expression construct, such as, e.g., pET29/BoNT/E-DiA, see, e.g., Example 1 is introduced into chemically competent E. coli BL21 (DE3) cells (Invitrogen, Inc, Carlsbad, Calif.) using a heat-shock transformation protocol. The heat-shock reaction is plated onto 1.5% Luria-Bertani agar plates (pH 7.0) containing 50 μg/mL of Kanamycin and is placed in a 37° C. incubator for overnight growth. Kanamycin-resistant colonies of transformed E. coli containing the expression construct, such as, e.g., pET29/BoNT/E-DiA are used to inoculate a baffled flask containing 3.0 mL of PA-0.5G media containing 50 μg/mL of Kanamycin which is then placed in a 37° C. incubator, shaking at 250 rpm, for overnight growth. The resulting overnight starter culture is in turn used to inoculate a 3 L baffled flask containing ZYP-5052 autoinducing media containing 50 μg/mL of Kanamycin at a dilution of 1:1000. Culture volumes ranged from about 600 mL (20% flask volume) to about 750 mL (25% flask volume). These cultures are grown in a 37° C. incubator shaking at 250 rpm for approximately 5.5 hours and are then transferred to a 16° C. incubator shaking at 250 rpm for overnight expression. Cells are harvested by centrifugation (4,000 rpm at 4° C. for 20-30 minutes) and are used immediately, or stored dry at −80° C. until needed.

### Example 3

### Purification and Quantification of Modified Clostridial Toxins

The following example illustrates methods useful for purification and quantification of any modified Clostridial toxins disclosed in the present specification.

For immobilized metal affinity chromatography (IMAC) protein purification, E. coli BL21 (DE3) cell pellets used to express a modified Clostridial toxin, as described in Example 2, are resuspended in Column Binding Buffer (25 mM N-(2-hydroxyethyl)piperazine-N′-(2-ethanesulfonic acid) (HEPES), pH 7.8; 500 mM sodium chloride; 10 mM imidazole; 2× Protease Inhibitor Cocktail Set III (EMD Biosciences-Calbiochem, San Diego Calif.); 5 units/mL of Benzonase (EMD Biosciences-Novagen, Madison, Wis.); 0.1% (v/v) Triton-X® 100, 4-octylphenol polyethoxylate; 10% (v/v) glycerol), and then are transferred to a cold Oakridge centrifuge tube. The cell suspension is sonicated on ice (10-12 pulses of 10 seconds at 40% amplitude with 60 seconds cooling intervals on a Branson Digital Sonifier) in order to lyse the cells and then is centrifuged (16,000 rpm at 4° C. for 20 minutes) to clarify the lysate. An immobilized metal affinity chromatography column is prepared using a 20 mL Econo-Pac column support (Bio-Rad Laboratories, Hercules, Calif.) packed with 2.5-5.0 mL of TALON™ SuperFlow Co²⁺ affinity resin (BD Biosciences-Clontech, Palo Alto, Calif.), which is then equilibrated by rinsing with 5 column volumes of deionized, distilled water, followed by 5 column volumes of Column Binding Buffer. The clarified lysate is applied slowly to the equilibrated column by gravity flow (approximately 0.25-0.3 mL/minute). The column is then washed with 5 column volumes of Column Wash Buffer (N-(2-hydroxyethyl)piperazine-N′-(2-ethanesulfonic acid) (HEPES), pH 7.8; 500 mM sodium chloride; 10 mM imidazole; 0.1% (v/v) Triton-X® 100, 4-octylphenol polyethoxylate; 10% (v/v) glycerol). The Clostridial toxin is eluted with 20-30 mL of Column Elution Buffer (25 mM N-(2-hydroxyethyl)piperazine-N′-(2-ethanesulfonic acid) (HEPES), pH 7.8; 500 mM sodium chloride; 500 mM imidazole; 0.1% (v/v) Triton-X® 100, 4-octylphenol polyethoxylate; 10% (v/v) glycerol) and is collected in approximately twelve 1 mL fractions. The amount of Clostridial toxin contained in each elution fraction is determined by a Bradford dye assay. In this procedure, 20 μL aliquots of each 1.0 mL fraction is combined with 200 μL of Bio-Rad Protein Reagent (Bio-Rad Laboratories, Hercules, Calif.), diluted 1 to 4 with deionized, distilled water, and then the intensity of the colorimetric signal is measured using a spectrophotometer. The five fractions with the strongest signal are considered the elution peak and are combined together. Total protein yield is determined by estimating the total protein concentration of the pooled peak elution fractions using bovine gamma globulin as a standard (Bio-Rad Laboratories, Hercules, Calif.).

For purification of a modified Clostridial toxin using a FPLC desalting column, a HiPrep™ 26/10 size exclusion column (Amersham Biosciences, Piscataway, N.J.) is pre-equilibrated with 80 mL of 4° C. Column Buffer (50 mM sodium phosphate, pH 6.5). After the column is equilibrated, a Clostridial toxin sample is applied to the size exclusion column with an isocratic mobile phase of 4° C. Column Buffer and at a flow rate of 10 mL/minute using a BioLogic DuoFlow chromatography system (Bio-Rad Laboratories, Hercules, Calif.). The desalted modified Clostridial toxin sample is collected as a single fraction of approximately 7-12 mL.

For purification of a modified Clostridial toxin using a FPLC ion exchange column, a Clostridial toxin sample that has been desalted following elution from an IMAC column is applied to a 1 mL Q1™ anion exchange column (Bio-Rad Laboratories, Hercules, Calif.) using a BioLogic DuoFlow chromatography system (Bio-Rad Laboratories, Hercules, Calif.). The sample is applied to the column in 4° C. Column Buffer (50 mM sodium phosphate, pH 6.5) and is eluted by linear gradient with 4° C. Elution Buffer (50 mM sodium phosphate, 1 M sodium chloride, pH 6.5) as follows: step 1, 5.0 mL of 5% Elution Buffer at a flow rate of 1 mL/minute; step 2, 20.0 mL of 5-30% Elution Buffer at a flow rate of 1 mL/minute; step 3, 2.0 mL of 50% Elution Buffer at a flow rate of 1.0 mL/minute; step 4, 4.0 mL of 100% Elution Buffer at a flow rate of 1.0 mL/minute; and step 5, 5.0 mL of 0% Elution Buffer at a flow rate of 1.0 mL/minute. Elution of Clostridial toxin from the column is monitored at 280, 260, and 214 nm, and peaks absorbing above a minimum threshold (0.01 au) at 280 nm are collected. Most of the Clostridial toxin will elute at a sodium chloride concentration of approximately 100 to 200 mM. Average total yields of Clostridial toxin will be determined by a Bradford assay.

Expression of a modified Clostridial toxin is analyzed by polyacrylamide gel electrophoresis. Samples purified using the procedure described above are added to 2×LDS Sample Buffer (Invitrogen, Inc, Carlsbad, Calif.) and are separated by MOPS polyacrylamide gel electrophoresis using NuPAGE® Novex 4-12% Bis-Tris precast polyacrylamide gels (Invitrogen, Inc, Carlsbad, Calif.) under denaturing, reducing conditions. Gels are stained with SYPRO® Ruby (Bio-Rad Laboratories, Hercules, Calif.) and the separated polypeptides are imaged using a Fluor-S MAX MultiImager (Bio-Rad Laboratories, Hercules, Calif.) for quantification of Clostridial toxin expression levels. The size and amount of the Clostridial toxin is determined by comparison to MagicMark™ protein molecular weight standards (Invitrogen, Inc, Carlsbad, Calif.).

Expression of modified Clostridial toxin is also analyzed by Western blot analysis. Protein samples purified using the procedure described above are added to 2×LDS Sample Buffer (Invitrogen, Inc, Carlsbad, Calif.) and are separated by MOPS polyacrylamide gel electrophoresis using NuPAGE® Novex 4-12% Bis-Tris precast polyacrylamide gels (Invitrogen, Inc, Carlsbad, Calif.) under denaturing, reducing conditions. Separated polypeptides are transferred from the gel onto polyvinylidene fluoride (PVDF) membranes (Invitrogen, Inc, Carlsbad, Calif.) by Western blotting using a Trans-Blot® SD semi-dry electrophoretic transfer cell apparatus (Bio-Rad Laboratories, Hercules, Calif.). PVDF membranes are blocked by incubating at room temperature for 2 hours in a solution containing 25 mM Tris-Buffered Saline (25 mM 2-amino-2-hydroxymethyl-1,3-propanediol hydrochloric acid (Tris-HCl) (pH 7.4), 137 mM sodium chloride, 2.7 mM potassium chloride), 0.1% TWEEN-20®, polyoxyethylene (20) sorbitan monolaureate, 2% bovine serum albumin, 5% nonfat dry milk. Blocked membranes are incubated at 4° C. for overnight in Tris-Buffered Saline TWEEN-20® (25 mM Tris-Buffered Saline, 0.1% TWEEN-20®, polyoxyethylene (20) sorbitan monolaureate) containing appropriate primary antibodies as a probe. Primary antibody probed blots are washed three times for 15 minutes each time in Tris-Buffered Saline TWEEN-20®. Washed membranes are incubated at room temperature for 2 hours in Tris-Buffered Saline TWEEN-20® containing an appropriate immunoglobulin G antibody conjugated to horseradish peroxidase as a secondary antibody. Secondary antibody-probed blots are washed three times for 15 minutes each time in Tris-Buffered Saline TWEEN-20®. Signal detection of the labeled Clostridial toxin are visualized using the ECL Plus™ Western Blot Detection System (Amersham Biosciences, Piscataway, N.J.) and are imaged with a Typhoon 9410 Variable Mode Imager (Amersham Biosciences, Piscataway, N.J.) for quantification of modified Clostridial toxin expression levels.

### Example 4

### Expression of Modified Clostridial Toxins in a Yeast Cell

The following example illustrates a procedure useful for expressing any of the modified Clostridial toxins disclosed in the present specification in a yeast cell.

To construct a suitable yeast expression construct encoding a modified Clostridial toxin, restriction endonuclease sites suitable for cloning an operably linked polynucleotide molecule into a pPIC A vector (Invitrogen, Inc, Carlsbad, Calif.) are incorporated into the 5′- and 3′ ends of the polynucleotide molecule encoding BoNT/E-DiA of SEQ ID NO: 50. This polynucleotide molecule is synthesized and a pUCBHB1/BoNT/E-DiA construct is obtained as described in Example 1. This construct is digested with restriction enzymes that 1) will excise the insert containing the open reading frame encoding BoNT/E-DiA; and 2) enable this insert to be operably-linked to a pPIC A vector. This insert is subcloned using a T4 DNA ligase procedure into a pPIC A vector that is digested with appropriate restriction endonucleases to yield pPIC A/BoNT/E-DiA. The ligation mixture is transformed into chemically competent E. coli DH5α cells (Invitrogen, Inc, Carlsbad, Calif.) using a heat shock method, plated on 1.5% low salt Luria-Bertani agar plates (pH 7.5) containing 25 μg/mL of Zeocin™, and placed in a 37° C. incubator for overnight growth. Bacteria containing expression constructs are identified as Zeocin™ resistant colonies. Candidate constructs are isolated using an alkaline lysis plasmid mini-preparation procedure and analyzed by restriction endonuclease digest mapping to determine the presence and orientation of the insert. This cloning strategy yielded a pPIC A expression construct comprising the polynucleotide molecule encoding the BoNT/E-DiA of SEQ ID NO: 50 operably-linked to a carboxyl-terminal c-myc and polyhistidine binding peptides.

A similar cloning strategy is used to make pPIC A expression constructs encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

To construct a yeast cell line expressing a modified Clostridial toxin, pPICZ A/BoNT/E-DiA is digested with a suitable restriction endonuclease (i.e., SacI, PmeI or BstXI) and the resulting linearized expression construct is transformed into an appropriate P. pastoris Mut^{S} strain KM71H using an electroporation method. The transformation mixture is plated on 1.5% YPDS agar plates (pH 7.5) containing 100 μg/mL of Zeocin™ and placed in a 28-30° C. incubator for 1-3 days of growth. Selection of transformants integrating the pPICZ A/BoNT/E-DiA at the 5′ AOX1 locus is determined by colony resistance to Zeocin™. Cell lines integrating a pPICZ A/BoNT/E-DiA construct is tested for BoNT/E-DiA expression using a small-scale expression test. Isolated colonies from test cell lines that have integrated pPICZ A/BoNT/E-DiA are used to inoculate 1.0 L baffled flasks containing 100 mL of MGYH media and grown at about 28-30° C. in a shaker incubator (250 rpm) until the culture reaches an OD₆₀₀=2-6 (approximately 16-18 hours). Cells are harvested by centrifugation (3,000×g at 22° C. for 5 minutes). To induce expression, the cell pellet is resuspended in 15 mL of MMH media and 100% methanol is added to a final concentration of 0.5%. Cultures are grown at about 28-30° C. in a shaker incubator (250 rpm) for six days. Additional 100% methanol is added to the culture every 24 hours to a final concentration of 0.5%. A 1.0 mL test aliquot is taken from the culture every 24 hours starting at time zero and ending at time 144 hours. Cells are harvested from the aliquots by microcentrifugation to pellet the cells and lysed using three freeze-thaw rounds consisting of −80° C. for 5 minutes, then 37° C. for 5 minutes. Lysis samples are added to 2×LDS Sample Buffer (Invitrogen, Inc, Carlsbad, Calif.) and expression from established cell lines is measured by Western blot analysis (as described in Example 8) using either anti-BoNT/E, anti-myc or anti-His antibodies in order to identify lines expressing BoNT/E-DiA. The P. pastoris Mut^{S} KM71H cell line showing the highest expression level of BoNT/E-DiA is selected for large-scale expression using commercial fermentation procedures. Procedures for large-scale expression are as outlined above except the culture volume is approximately 2.5 L MGYH media grown in a 5 L BioFlo 3000 fermentor and concentrations of all reagents will be proportionally increased for this volume. A similar procedure can be used to express a pPICZ A construct encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

BoNT/E-DiA is purified using the IMAC procedure, as described in Example 3. Expression from each culture is evaluated by a Bradford dye assay, polyacrylamide gel electrophoresis and Western blot analysis (as described in Example 3) in order to determine the amounts of BoNT/E-DiA produced.

### Example 5

### Expression of Modified Clostridial Toxins in an Insect Cell

The following example illustrates a procedure useful for expressing any of the modified Clostridial toxins disclosed in the present specification in an insect cell.

To construct suitable an insect expression construct encoding a modified Clostridial toxin, restriction endonuclease sites suitable for cloning an operably linked polynucleotide molecule into a pBACgus3 vector (EMD Biosciences-Novagen, Madison, Wis.) are incorporated into the 5′- and 3′ ends of the polynucleotide molecule encoding BoNT/E-DiA of SEQ ID NO: 50. This polynucleotide molecule is synthesized and a pUCBHB1/BoNT/E-DiA construct is obtained as described in Example 1. This construct is digested with restriction enzymes that 1) will excise the insert containing the open reading frame encoding BoNT/E-DiA; and 2) enable this insert to be operably-linked to a pBACgus3 vector. This insert is subcloned using a T4 DNA ligase procedure into a pBACgus3 vector that is digested with appropriate restriction endonucleases to yield pBACgus3/BoNT/E-DiA. The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yielded a pBACgus3 expression construct comprising the polynucleotide molecule encoding the BoNT/E-DiA of SEQ ID NO: 50 operably linked to an amino-terminal gp64 signal peptide and a carboxyl-terminal, Thrombin cleavable, polyhistidine affinity binding peptide.

A similar cloning strategy is used to make pBACgus3 expression constructs encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

To express a modified Clostridial toxin using a baculoviral expression system, about 2.5×10⁶ Sf9 cells are plated in four 60 mm culture dishes containing 2 mL of BacVector® Insect media (EMD Biosciences-Novagen, Madison, Wis.) and incubated for approximately 20 minutes in a 28° C. incubator. For each transfection, a 50 μL transfection solution is prepared in a 6 mL polystyrene tube by adding 25 μL of BacVector® Insect media containing 100 ng of a pBACgus3 construct encoding a modified Clostridial toxin, such as, e.g., pBACgus3/BoNT/E-DiA, and 500 ng TlowE transfer plasmid to 25 μL of diluted Insect GeneJuice® containing 5 μL Insect GeneJuice® (EMD Biosciences-Novagen, Madison, Wis.) and 20 μL nuclease-free water and this solution is incubated for approximately 15 minutes. After the 15 minute incubation, add 450 μL BacVector® media to the transfection solution and mix gently. Using this stock transfection solution as the 1/10 dilution make additional transfection solutions of 1/50, 1/250 and 1/1250 dilutions. Add 100 μL of a transfection solution to the Sf9 cells from one of the four 60 mm culture dishes, twice washed with antibiotic-free, serum-free BacVector® Insect media and incubate at 22° C. After one hour, add 6 mL of 1% BacPlaque agarose-BacVector® Insect media containing 5% bovine serum albumin. After the agarose is solidified, add 2 mL BacVector® Insect media containing 5% bovine serum albumin to the transfected cells and transfer the cells to a 28° C. incubator for 3-5 days until plaques are visible. After 3-5 days post-transfection, plaques in the monolayer will be stained for 11-glucuronidase reporter gene activity to test for the presence of recombinant virus plaques containing pBACgus3/BoNT/E-DiA by incubating the washed monolayer with 2 mL of BacVector® Insect media containing 30 μL of 20 mg/mL X-Gluc Solution (EMD Biosciences-Novagen, Madison, Wis.) for approximately 2 hours in a 28° C. incubator.

After identifying candidate recombinant virus plaques, several candidate virus plaques are eluted and plaque purified. To elute a recombinant virus, transfer a plug containing a recombinant virus plaque with a sterile Pasteur pipet to 1 mL BacVector® Insect media (EMD Biosciences-Novagen, Madison, Wis.) in a sterile screw-cap vial. Incubate the vial for approximately 2 hours at 22° C. or for approximately 16 hours at 4° C. For each recombinant virus plaque, 2.5×10⁵ Sf9 cells are plated in 35 mm culture dishes containing 2 mL of BacVector® Insect media (EMD Biosciences-Novagen, Madison, Wis.) and incubated for approximately 20 minutes in a 28° C. incubator. Remove the media and add 200 μL of eluted recombinant virus. After one hour, add 2 mL of 1% BacPlaque agarose-BacVector® Insect media containing 5% bovine serum albumin. After the agarose is solidified, add 1 mL BacVector® Insect media containing 5% bovine serum albumin to the transfected cells and transfer the cells to a 28° C. incubator for 3-5 days until plaques are visible. After 3-5 days post-transfection, plaques in the monolayer will be stained for R-glucuronidase reporter gene activity to test for the presence of recombinant virus plaques containing pBACgus3/BoNT/E-DiA by incubating the washed monolayer with 2 mL of BacVector® Insect media containing 30 μL of 20 mg/mL X-Gluc Solution (EMD Biosciences-Novagen, Madison, Wis.) for approximately 2 hours in a 28° C. incubator.

To prepare a seed stock of virus, elute a recombinant virus by transferring a plug containing a recombinant virus plaque with a sterile Pasteur pipet to 1 mL BacVector® Insect media (EMD Biosciences-Novagen, Madison, Wis.) in a sterile screw-cap vial. Incubate the vial for approximately 16 hours at 4° C. Approximately 5×10⁵ Sf9 cells are plated in T-25 flask containing 5 mL of BacVector® Insect media (EMD Biosciences-Novagen, Madison, Wis.) and are incubated for approximately 20 minutes in a 28° C. incubator. Remove the media and add 300 μL of eluted recombinant virus. After one hour, add 5 mL BacVector® Insect media containing 5% bovine serum albumin to the transfected cells and transfer the cells to a 28° C. incubator for 3-5 days until the majority of cells become unattached and unhealthy. The virus is harvested by transferring the media to 15 mL snap-cap tubes and centrifuging tubes at 1000×g for 5 minutes to remove debris. The clarified supernatant is transferred to fresh 15 mL snap-cap tubes and are stored at 4° C.

To prepare a high titer stock of virus, approximately 2×10⁷ Sf9 cells are plated in T-75 flask containing 10 mL of BacVector® Insect media (EMD Biosciences-Novagen, Madison, Wis.) and are incubated for approximately 20 minutes in a 28° C. incubator. Remove the media and add 500 μL of virus seed stock. After one hour, add 10 mL BacVector® Insect media containing 5% bovine serum albumin to the transfected cells and transfer the cells to a 28° C. incubator for 3-5 days until the majority of cells become unattached and unhealthy. The virus is harvested by transferring the media to 15 mL snap-cap tubes and centrifuging tubes at 1000×g for 5 minutes to remove debris. The clarified supernatant is transferred to fresh 15 mL snap-cap tubes and are stored at 4° C. High titer virus stocks should contain approximately 2×10⁸ to 3×10⁹ pfu of baculovirus.

To express gp64-BoNT/E-DiA using a baculoviral expression system, about 1.25×10⁸ Sf9 cells are seeded in a 1 L flask containing 250 mL of BacVector® Insect media and are grown in an orbital shaker (150 rpm) to a cell density of approximately 5×10⁸. The culture is inoculated with approximately 2.5×10⁹ of high titer stock recombinant baculovirus and incubated for approximately 48 hours in a 28° C. orbital shaker (150 rpm). Media is harvested by transferring the media to tubes and centrifuging tubes at 500×g for 5 minutes to remove debris. Media samples are added to 2×LDS Sample Buffer (Invitrogen, Inc, Carlsbad, Calif.) and expression is measured by Western blot analysis (as described in Example 8) using either anti-BoNT/A or anti-His antibodies in order to identify baculoviral stocks expressing BoNT/E-DiA. A similar procedure can be used to express a pBACgus3 construct encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

BoNT/E-DiA is purified using the IMAC procedure, as described in Example 3. Expression from each culture is evaluated by a Bradford dye assay, polyacrylamide gel electrophoresis and Western blot analysis (as described in Example 3) in order to determine the amounts of BoNT/E-DiA produced.

### Example 6

### Expression of Modified Clostridial Toxins in a Mammalian Cell

The following example illustrates a procedure useful for expressing any of the modified Clostridial toxins disclosed in the present specification in a mammalian cell.

To construct a suitable mammalian expression construct encoding a modified Clostridial toxin, restriction endonuclease sites suitable for cloning an operably linked polynucleotide molecule into a pSecTag2 vector (Invitrogen, Inc, Carlsbad, Calif.) are incorporated into the 5′- and 3′ ends of the polynucleotide molecule encoding BoNT/E-DiA of SEQ ID NO: 50. This polynucleotide molecule is synthesized and a pUCBHB1/BoNT/E-DiA construct is obtained as described in Example 1. This construct is digested with restriction enzymes that 1) will excise the insert containing the open reading frame encoding BoNT/E-DiA; and 2) enable this insert to be operably-linked to a pSecTag2 vector. This insert is subcloned using a T4 DNA ligase procedure into a pSecTag2 vector that is digested with appropriate restriction endonucleases to yield pSecTag2/BoNT/E-DiA. The ligation mixture is transformed into chemically competent E. coli DH5α cells (Invitrogen, Inc, Carlsbad, Calif.) using a heat shock method, plated on 1.5% Luria-Bertani agar plates (pH 7.0) containing 100 μg/mL of Ampicillin, and placed in a 37° C. incubator for overnight growth. Bacteria containing expression constructs are identified as Ampicillin resistant colonies. Candidate constructs are isolated using an alkaline lysis plasmid mini-preparation procedure and analyzed by restriction endonuclease digest mapping to determine the presence and orientation of the insert. This cloning strategy yielded a pSecTag2 expression construct comprising the polynucleotide molecule encoding the BoNT/E-DiA of SEQ ID NO: 50 operably-linked to a carboxyl-terminal c-myc and polyhistidine binding peptides.

A similar cloning strategy is used to make pSecTag2 expression constructs encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

To transiently express modified Clostridial toxin in a cell line, about 1.5×10⁵ SH-SY5Y cells are plated in a 35 mm tissue culture dish containing 3 mL of complete Dulbecco's Modified Eagle Media (DMEM), supplemented with 10% fetal bovine serum (FBS), 1× penicillin/streptomycin solution (Invitrogen, Inc, Carlsbad, Calif.) and 1×MEM non-essential amino acids solution (Invitrogen, Inc, Carlsbad, Calif.), and grown in a 37° C. incubator under 5% carbon dioxide until cells reach a density of about 5×10⁵ cells/ml (6-16 hours). A 500 μL transfection solution is prepared by adding 250 μL of OPTI-MEM Reduced Serum Medium containing 15 μL of LipofectAmine 2000 (Invitrogen, Carlsbad, Calif.) incubated at room temperature for 5 minutes to 250 μL of OPTI-MEM Reduced Serum Medium containing 5 μg of a pSecTag2 expression construct encoding a modified Clostridial toxin, such as, e.g., pSecTag2/BoNT/E-DiA. This transfection is incubated at room temperature for approximately 20 minutes. The complete, supplemented DMEM media is replaced with 2 mL of OPTI-MEM Reduced Serum Medium and the 500 μL transfection solution is added to the SH-SY5Y cells and the cells are incubated in a 37° C. incubator under 5% carbon dioxide for approximately 6 to 18 hours. Transfection media is replaced with 3 mL of fresh complete, supplemented DMEM and the cells are incubated in a 37° C. incubator under 5% carbon dioxide for 48 hours. Both media and cells are collected for expression analysis of BoNT/E-DiA. Media is harvested by transferring the media to 15 mL snap-cap tubes and centrifuging tubes at 500×g for 5 minutes to remove debris. Cells are harvested by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen, Inc, Carlsbad, Calif.) and expression is measured by Western blot analysis (as described in Example 5) using either anti-BoNT/E, anti-c-myc or anti-His antibodies in order to identify pSecTag2 constructs expressing BoNT/E-DiA. A similar procedure can be used to transiently express a pSecTag2 construct encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

To generate a stably-integrated cell line expressing a modified Clostridial toxin, approximately 1.5×10⁵ SH-SY5Y cells are plated in a 35 mm tissue culture dish containing 3 mL of complete DMEM, supplemented with 10% FBS, 1× penicillin/streptomycin solution (Invitrogen, Inc, Carlsbad, Calif.) and 1×MEM non-essential amino acids solution (Invitrogen, Inc, Carlsbad, Calif.), and grown in a 37° C. incubator under 5% carbon dioxide until cells reach a density of about 5×10⁵ cells/ml (6-16 hours). A 500 μL transfection solution is prepared by adding 250 μL of OPTI-MEM Reduced Serum Medium containing 15 μL of LipofectAmine 2000 (Invitrogen, Carlsbad, Calif.) incubated at room temperature for 5 minutes to 250 μL of OPTI-MEM Reduced Serum Medium containing 5 μg of a pSecTag2 expression construct encoding a modified Clostridial toxin, such as, e.g., pSecTag2/BoNT/E-DiA. This transfection solution is incubated at room temperature for approximately 20 minutes. The complete, supplemented DMEM media is replaced with 2 mL of OPTI-MEM Reduced Serum Medium and the 500 μL transfection solution is added to the SH-SY5Y cells and the cells are incubated in a 37° C. incubator under 5% carbon dioxide for approximately 6 to 18 hours. Transfection media is replaced with 3 mL of fresh complete, supplemented DMEM and cells are incubated in a 37° C. incubator under 5% carbon dioxide for approximately 48 hours. Media is replaced with 3 mL of fresh complete DMEM, containing approximately 5 μg/mL of Zeocin™ 10% FBS, 1× penicillin/streptomycin solution (Invitrogen, Inc, Carlsbad, Calif.) and 1×MEM non-essential amino acids solution (Invitrogen, Inc, Carlsbad, Calif.). Cells are incubated in a 37° C. incubator under 5% carbon dioxide for approximately 3-4 weeks, with old media being replaced with fresh Zeocin™-selective, complete, supplemented DMEM every 4 to 5 days. Once Zeocin™-resistant colonies are established, resistant clones are replated to new 35 mm culture plates containing fresh complete DMEM, supplemented with approximately 5 μg/mL of Zeocin™, 10% FBS, 1× penicillin/streptomycin solution (Invitrogen, Inc, Carlsbad, Calif.) and 1×MEM non-essential amino acids solution (Invitrogen, Inc, Carlsbad, Calif.), until these cells reach a density of 6 to 20×10⁵ cells/mL. To test for expression of BoNT/E-DiA from SH-SY5Y cell lines that have stably-integrated a pSecTag2/BoNT/E-DiA, approximately 1.5×10⁵ SH-SY5Y cells from each cell line are plated in a 35 mm tissue culture dish containing 3 mL of Zeocin™-selective, complete, supplemented DMEM and grown in a 37° C. incubator under 5% carbon dioxide until cells reach a density of about 5×10⁵ cells/ml (6-16 hours). Media is replaced with 3 mL of fresh Zeocin™-selective, complete, supplemented DMEM and cells are incubated in a 37° C. incubator under 5% carbon dioxide for 48 hours. Both media and cells are collected for expression analysis of BoNT/E-DiA-c-myc-His. Media is harvested by transferring the media to 15 mL snap-cap tubes and centrifuging tubes at 500×g for 5 minutes to remove debris. Cells are harvest by rinsing cells once with 3.0 mL of 100 mM phosphate-buffered saline, pH 7.4 and lysing cells with a buffer containing 62.6 mM 2-amino-2-hydroxymethyl-1,3-propanediol hydrochloric acid (Tris-HCl), pH 6.8 and 2% sodium lauryl sulfate (SDS). Both media and cell samples are added to 2×LDS Sample Buffer (Invitrogen, Inc, Carlsbad, Calif.) and expression is measured by Western blot analysis (as described in Example 5) using either anti-BoNT/A, anti-c-myc or anti-His antibodies in order to identify SH-SY5Y cell lines expressing BoNT/E-DiA. The established SH-SY5Y cell line showing the highest expression level of BoNT/E-DiA is selected for large-scale expression using 3 L flasks. Procedures for large-scale expression are as outlined above except the starting volume is approximately 800-1000 mL of complete DMEM and concentrations of all reagents are proportionally increased for this volume. A similar procedure can be used to stably express a pSecTag2 construct encoding BoNT/E-DiB, BoNT/E-DiF, BoNT/E-Ba, BoNT/E-DiT, BoNT/B-DiA, BoNT/C1-DiA, BoNT/D-DiA, BoNT/F-DiA, BoNT/G-DiA, TeNT-DiA, BaNT-DiA, and BuNT-DiA.

BoNT/E-DiA is purified using the IMAC procedure, as described in Example 3. Expression from each culture is evaluated by a Bradford dye assay, polyacrylamide gel electrophoresis and Western blot analysis (as described in Example 3) in order to determine whether the amounts of BoNT/E-DiA produced.

### Example 7

### Construction of Clostridial Toxins for Recombinant Expression

A polynucleotide molecule based on BoNT/A (SEQ ID NO: 1) will be synthesized and cloned into a pUCBHB1 vector as described in Example 1. If so desired, expression optimization to a different organism, such as, e.g., a bacteria, a yeast strain, an insect cell-line or a mammalian cell line, can be done as described above, see, e.g., Steward, supra, (Feb. 2, 2006); and Steward, supra, (Feb. 16, 2006). A similar cloning strategy will be used to make pUCBHB1 cloning constructs BoNT/B (SEQ ID NO: 2), BoNT/C1 (SEQ ID NO: 3), BoNT/D (SEQ ID NO: 4), BoNT/E (SEQ ID NO: 5), BoNT/F (SEQ ID NO: 6), BoNT/G (SEQ ID NO: 7), TeNT (SEQ ID NO: 8), BaNT (SEQ ID NO: 9), and BuNT (SEQ ID NO: 10).

To construct pET29/BoNT/A, a pUCBHB1/BoNT/A construct will be digested with restriction endonucleases that 1) will excise the polynucleotide molecule encoding the open reading frame of BoNT/A; and 2) will enable this polynucleotide molecule to be operably-linked to a pET29 vector (EMD Biosciences-Novagen, Madison, Wis.). This insert will be subcloned using a T4 DNA ligase procedure into a pET29 vector that is digested with appropriate restriction endonucleases to yield pET29/BoNT/A. The ligation mixture will be transformed into chemically competent E. coli DH5α cells (Invitrogen, Inc, Carlsbad, Calif.) using a heat shock method, will be plated on 1.5% Luria-Bertani agar plates (pH 7.0) containing 50 μg/mL of Kanamycin, and will be placed in a 37° C. incubator for overnight growth. Bacteria containing expression constructs will be identified as Kanamycin resistant colonies. Candidate constructs will be isolated using an alkaline lysis plasmid mini-preparation procedure and will be analyzed by restriction endonuclease digest mapping to determine the presence and orientation of the insert. This cloning strategy will yield a pET29 expression construct comprising the polynucleotide molecule encoding the BoNT/A operably-linked to a carboxyl terminal polyhistidine affinity binding peptide. A similar cloning strategy will be used to make pET29 expression constructs for other modified BoNT/B, BoNT/C1, BoNT/D, BoNT/E, BoNT/F, BoNT/G-TEV, TeNT-TEV, BaNT, or BuNT.

### Example 8

### Expression and Purification of Recombinant Clostridial Toxins in a Bacterial Cell

The following example illustrates a procedure useful for recombinantly expressing any of the Clostridial toxins disclosed in the present specification in a bacterial cell.

An expression construct, such as, e.g., pET29/BoNT/A, see, e.g., Example 7 is introduced into chemically competent E. coli BL21 (DE3) cells (Invitrogen, Inc, Carlsbad, Calif.) using a heat-shock transformation protocol. The heat-shock reaction is plated onto 1.5% Luria-Bertani agar plates (pH 7.0) containing 50 μg/mL of Kanamycin and is placed in a 37° C. incubator for overnight growth. Kanamycin-resistant colonies of transformed E. coli containing the expression construct, such as, e.g., pET29/BoNT/iA are used to inoculate a baffled flask containing 3.0 mL of PA-0.5G media containing 50 μg/mL of Kanamycin which is then placed in a 37° C. incubator, shaking at 250 rpm, for overnight growth. The resulting overnight starter culture is in turn used to inoculate a 3 L baffled flask containing ZYP-5052 autoinducing media containing 50 μg/mL of Kanamycin at a dilution of 1:1000. Culture volumes ranged from about 600 mL (20% flask volume) to about 750 mL (25% flask volume). These cultures are grown in a 37° C. incubator shaking at 250 rpm for approximately 5.5 hours and are then transferred to a 16° C. incubator shaking at 250 rpm for overnight expression. Cells are harvested by centrifugation (4,000 rpm at 4° C. for 20-30 minutes) and are used immediately, or stored dry at −80° C. until needed.

Recombinantly-expressed BoNT/A is purified using the IMAC procedure, as described in Example 3. Expression from each culture is evaluated by a Bradford dye assay, polyacrylamide gel electrophoresis and Western blot analysis (as described in Example 3) in order to determine the amounts of recombinantly-expressed BoNT/A produced.

To activate purified, recombinantly-expressed BoNT/A, approximately 30 μg of purified, recombinantly-expressed BoNT/A will be incubated with 3 μg of di-chain loop protease of SEQ ID NO: 33 in 20 mM Tris-HCl, pH 8.0 with 200 mM NaCl. Following incubation at 23° C. for 2 hours, the nicking reaction will be quenched by addition of 1× Protease Inhibitor Cocktail Set III (CalBiochem; 1× inhibitor contains 1 mM AEBSF, 0.8 μM Aprotinin, 50 μM Bestatin, 15 μM E-64, 20 μM Leupeptin, and 10 μM Pepstatin A). The samples may be flash frozen in liquid nitrogen and immediately stored at −80° C.

Although aspects of the present invention have been described with reference to the disclosed embodiments, one skilled in the art will readily appreciate that the specific examples disclosed are only illustrative of these aspects and in no way limit the present invention. Various modifications can be made without departing from the spirit of the present invention.

What is claimed:

1. A modified Clostridial toxin comprising an exogenous BoNT/A di-chain loop region including a BoNT/A di-chain protease cleavage site;
wherein the Clostridial toxin is a BoNT/E, and
wherein the BoNT/A di-chain loop region replaces an endogenous Clostridial toxin di-chain loop region.

2. A polynucleotide molecule encoding a modified Clostridial toxin according to claim 1.

3. A method of producing a modified Clostridial toxin comprising the step of expressing in a cell a polynucleotide molecule according to claim 2, wherein expression from the polynucleotide molecule produces the encoded modified Clostridial toxin.

4. A method of producing a modified Clostridial toxin comprising the steps of:
a. introducing into a cell a polynucleotide molecule as defined in claim 2; and
b. expressing the polynucleotide molecule, wherein expression from the polynucleotide molecule produces the encoded modified Clostridial toxin.
